(19) United States
(12) Patent Application Publication
(10) Pub. No.: US 2026/0184748 A1
(43) Pub. Date: Jul. 2, 2026

(54) COMPOSITIONS COMPRISING OCTADECANEUROPEPTIDES (ODN) AND SYNTHETIC DERIVATIVES THEREOF AND METHODS OF USE FOR MODULATION OF FOOD INTAKE, OBESITY, BODY WEIGHT, NAUSEA, AND EMESIS

(71) Applicants: The Trustees of the University of Pennsylvania, Philadelphia, PA (US); Syracuse University, Syracuse, NY (US)

(72) Inventors: Matthew R. Hayes, Lansdale, PA (US); Robert P. Doyle, Manlius, NY (US); Caroline Geisler, West Deptford, NJ (US); Kylie S. Chichura, Norwood, MA (US); Benjamin C. Reiner, West Chester, PA (US); Richard C. Crist, Lansdale, PA (US)

(73) Assignees: The Trustees of the University of Pennsylvania, Philadelphia, PA (US); Syracuse University, Syracuse, NY (US)

(21) Appl. No.: 19/131,005

(22) PCT Filed: Nov. 20, 2023
(86) PCT No.: PCT/US2023/080550

§ 371 Date: May 19, 2025

Related U.S. Application Data
(60) Provisional application No. 63/384,306, filed on Nov. 18, 2022.

(51) Int. Cl. C07K 14/435; A61K 38/00; A61P 3/04

(52) U.S. Cl. CPC C07K 14/435; A61P 3/04; A61K 38/00

(57) ABSTRACT

Compositions and methods for the management and treatment of obesity, metabolic disorders, nausea and emesis using a ODN peptides and synthetic derivatives thereof are disclosed.

### CROSS REFERENCE TO RELATED APPLICATION

This application claims the benefit of U.S. Provisional Patent Application No. 63/384,306, filed Nov. 18, 2022, the entire contents be incorporated herein by reference as though set forth in full.

### INCORPORATION-BY-REFERENCE OF MATERIAL SUBMITTED IN ELECTRONIC FORM

The Contents of the electronic sequence listing (UPNK-114PCT.xml; Size: 35,858 bytes; and Date of Creation: Nov. 20, 2023) is herein incorporated by reference in its entirety.

### FIELD OF THE INVENTION

The present invention relates to the fields of neuroactive compounds and methods of use thereof for the management and treatment of food intake, obesity, nausea and emesis. More specifically, certain endozepines, e.g., octadecaneuropeptide (ODN) and its precursor diazepam-binding inhibitor (DBI) peptide, and derivatives thereof are provided which act in the central nervous system to not only control food intake but also protect neurons and astrocytes from programmed cell death by reducing inflammation, apoptosis and oxidative stress.

### BACKGROUND OF THE INVENTION

Several publications and patent documents are cited throughout the specification in order to describe the state of the art to which this invention pertains. Each of these citations is incorporated by reference herein as though set forth in full.

Obesity affects more than 40% of the U.S. population. The excess fat in obesity was originally thought to be harmless (benign). However, data show that excess fat causes chemical changes in your blood that increase your heart disease risk. When your fat cells become enlarged, they give off hormones that produce chronic inflammation. Inflammation can lead to the body no longer using insulin efficiently, thereby producing insulin resistance. Insulin resistance is associated with trouble regulating blood sugar levels, which can result in metabolic syndrome. This condition is associated with several risk factors that increase your chance of developing heart disease, such as: High blood lipids (high LDL cholesterol, high total cholesterol and high triglycerides), high blood pressure (hypertension), high blood sugar (hyperglycemia), and low HDL cholesterol. Obesity increases other heart disease risk factors, including sleep disorders and type 2 diabetes.

Overweight and obesity are defined by the World Health Organization as abnormal or excessive fat that accumulates and presents a risk to health. Obesity is measured in body mass index (BMI), which is a person's weight (in kilograms) divided by the square of his or her height (in meters). A person with a BMI of 30 or more is generally considered obese. A person with a BMI equal to or more than 25 is considered overweight. Losing 5% to 10% of body weight can lower the risk factors for heart disease while small lifestyle changes can help improve metabolic syndrome, which lessens your heart disease risk.

Lifestyle interventions on diet and physical activity are the first option for the management of obesity and overweight, but efficacy can be limited, and weight regain is common. Bariatric surgery can be highly effective for weight loss in severely obese or high-risk patients, but its use is limited by its invasive nature, cost, risk of perioperative adverse events including perioperative death. While a few drugs have demonstrated efficacy in weight-reduction, pharmacotherapy for the treatment of obesity is limited by the modest weight loss induced by most drugs, development of dependency, side effect profile of some agents, contraindications, low compliance, and barriers to treatment including under-prescription.

Clearly, a need exists for improved, non-toxic pharmaceuticals for the treatment of obesity for sustained and manageable control of food intake and a reduction in the adverse health consequences associated with such pharmaceuticals, including as nausea and emesis.

### SUMMARY OF THE INVENTION

In accordance with the invention isolated and/or purified octadecaneuropeptides (ODN) or synthetic derivative thereof, comprising at least one amino acid sequence selected from SEQ ID NOS: 1 to SEQ ID NO: 21 or functional fragment thereof, or a sequence having at least 95% identity thereto arc provided, which effectively modulate food intake, and body weight. In preferred embodiments the octadecaneuropeptide inhibits emesis and nausea.

In other embodiments, the octadecaneuropeptide comprises one or more modified amino acids selected from those listed in Table 2, and, or, one or more of Gln(alkyn), Ala(Alkyn), Gly(Alkyn) and Lys(N3). Certain modified amino acids are suitable for bioconjugation of desired moieties, including lipids, cell penetrating peptides, cell targeting peptides, e.g., transferrin, Fc-peptide and reporter molecules, e.g., fluorescent tagging. In a preferred embodiment the octadecaneuropeptide is SEQ ID NO:3 or a synthetic derivative of TDN of SEQ ID NO:3 or a functional modified variant thereof.

In another aspect, a composition comprising any one of the octadecaneuropeptides described above in a pharmaceutically acceptable carrier is provided. In another aspect, isolated nucleic acids encoding amino acid sequences of SEQ ID NOS: 1 to SEQ ID NO: 21 are also disclosed. In other aspects the isolated nucleic is present in a vector for robust expression and production in an organism of interest.

In yet another embodiment, a nucleic acid sequence that encodes any one of the octadecaneuropeptides having amino acid SEQ ID NOS: 1 to SEQ ID NO: 21 is disclosed. In other embodiments, the nucleic acid encodes the polypeptide of SEQ ID NO: 3. The nucleic acids of the invention may be in a vector and/or encapsulated in a liposome or an extracellular vesicle or affixed to a nanoparticle or lipid nanoparticle to facilitate delivery in vivo. Vectors include without limitation, a plasmid vector, a lentiviral vector, an AAV vector, AAV9, and AAV8 viral vector. Also disclosed is a composition comprising the nucleic acid encoding the octadecaneuropeptide described above in a pharmaceutically acceptable carrier or buffer.

In a preferred embodiment, methods for treating a metabolic disease or disorder in a subject in need thereof, comprising administering an effective amount of the composition comprising any one of the octadecaneuropeptides described above in a pharmaceutically acceptable carrier are provided. In certain approaches, the composition is effective inhibit relaxin-3 receptor producing anorexic effects. In other approaches, the composition functions to modulate one or more of glucose tolerance, regulation of the counterregulatory response and attenuate 5-tg induced hyperglycemia, insulin sensitivity, and weight loss. In other approaches the composition is able to cross the blood brain barrier and penetrate into the brain. Routes of administration can include any one of systemic, parenteral, transdermal patch, intramuscular, subcutaneous, intracerebral, oral, and buccal. The skilled clinician is aware of the many available routes of administration. Metabolic diseases or disorders include, without limitation obesity, diabetes mellitus, dyslipidemia, insulin resistance, hepatic steatosis, hypercholesterolemia, and non-alcoholic fatty liver. In certain embodiments, the methods can also comprise administering a second therapeutic agent that treats or inhibits progress at least one disorder selected from obesity, diabetes mellitus, dyslipidemia, insulin resistance, hepatic steatosis, hypercholesterolemia, and non-alcoholic fatty liver. In preferred embodiments the weight of the patient decreases following administration of the peptide. The methods described above can also include assessing the patient for a reduction in symptoms associated with at least one disorder selected from obesity, diabetes mellitus, dyslipidemia, insulin resistance, hepatic steatosis, hypercholesterolemia, and non-alcoholic fatty liver.

### BRIEF DESCRIPTION OF THE DRAWINGS

FIGS. 1A-1D: Central ODN dose dependently suppresses food intake in chow and HFD rats. Effect of 4th ventricle ODN (0.2, 2, or 20 μg/2 μL in aCSF) treatment on 24 h food intake (grams; FIG. 1A, FIG. 1C) and body weight change (FIG. 1B, FIG. 1D) in chow and HFD fed rats. All data presented as mean±SEM.

FIGS. 2A-2T. Meal pattern data following central ODN administration in chow fed rats. Effect of 4th ventricle ODN (0.2, 2, or 20 μg/2 μL in aCSF) treatment on food intake (grams; FIGS. 2A-2D), number of meals (FIGS. 2E-2H), time spent eating meals (seconds; FIGS. 21-2L), meal length (seconds/meal; FIGS. 2M-2P), and meal size (grams/meal; FIGS. 2Q-2T) at time intervals 1, 6, 6-12, and 12-24 hours after administration. All data presented as mean±SEM.

FIGS. 3A-3E. Heat map representation of bout and meal parameters during the first 3 hours following central ODN administration in chow fed rats. Effect of 4th ventricle ODN (0.2, 2, or 20 μg/2 μL in aCSF) on food intake (grams; FIG. 3A), number of bouts (FIG. 3B), time spent eating bouts (seconds; FIG. 3C), number of meals (FIG. 3D), and time spent eating meals (FIG. 3E). * Indicates difference from Veh (P=0.05). All data presented as mean±SEM.

FIGS. 4A-4T. Meal pattern data following central ODN administration in HFD fed rats. Effect of 4th ventricle ODN (0.2, 2, or 20 μg/2 μL in aCSF) treatment on food intake (grams; FIGS. 4A-4D), number of meals (FIGS. 4E-4H), time spent eating meals (seconds; FIGS. 4I-4L), meal length (seconds/meal; FIGS. 4M-4P), and meal size (grams/meal; FIGS. 4Q-4T) at time intervals 1, 6, 6-12, and 12-24 hours after administration. All data presented as mean±SEM.

FIGS. 5A-5E. Heat map representation of bout and meal parameters during the 24 hours following central ODN administration in HFD fed rats. Effect of 4th ventricle ODN (0.2, 2, or 20 g/2 μL in aCSF) on food intake (grams; FIG. 5A), number of bouts (FIG. 5B), time spent eating bouts (seconds; FIG. 5C), number of meals (FIG. 5D), and time spent eating meals (FIG. 5E). * Indicates difference from Veh (P=0.05). All data presented as mean±SEM.

FIGS. 6A-6H. First feeding event data following central ODN administration in chow and HFD fed rats. Effect of 4th ventricle ODN (0.2, 2, or 20 μg/2 μL in aCSF) on latency to the first meal (seconds; FIG. 6A, FIG. 6E), size of the first meal (grams; FIG. 6B, FIG. 6F), latency to first bout (seconds; FIG. 6C, FIG. 6G), size of first bout (grams, FIG. 6D, FIG. 6H) in chow and HFD fed rats. All data presented as mean±SEM.

FIGS. 7A-7C. Effect of hindbrain 4th ventricle recombinant DBI protein on meal pattern food intake in chow and HFD rats. Central recombinant DBI protein suppresses food intake in chow fed rats. Effect of 4th ventricle recombinant DBI protein (20 μg/2 μL) on 24 h food intake in chow (FIG. 7A) and HFD (FIG. 7B) fed rats and body weight change in chow and HFD rats (FIG. 7C). All data presented as mean±SEM.

FIGS. 8A-8E. Heat map representation of bout and meal parameters during the 24 hours following central recombinant DBI protein administration in chow fed rats. Effect of 4th ventricle recDBI (20 μg/2 μL in aCSF) on food intake (grams; FIG. 8A), number of bouts (FIG. 8B), time spent eating bouts (seconds; FIG. 8C), number of meals (FIG. 8D), and time spent eating meals (FIG. 8E). * Indicates difference from Veh (P=0.05). All data presented as mean±SEM.

FIGS. 9A-9F. Pretreatment with an antibody against DBI attenuates central exendin-4 induced hypophagia in chow fed rats. Rats were pretreated 4th ventricle with a DBI antibody (AB 3 μg/3 μL) or vehicle followed by 4th ventricle treatment with ODN (20 μg/2 μL), Ex-4 (0.3 μg/2 μL), or vehicle. 24-hour food intake in chow (FIG. 9A) and HFD (FIG. 9B) fed rats, kaolin intake in chow (FIG. 9C) and HFD (FIG. 9D) fed rats, and body weight change in chow (FIG. 9E) and HFD (FIG. 9F) fed rats following treatments. #Indicates difference from Veh/Veh (P=0.05). All data presented as mean±SEM.

FIGS. 10A-10F. Pretreatment with an ODN antagonist attenuates central exendin-4 induced hypophagia. Rats were pretreated 4th ventricle with an ODN antagonist (AntOP 200 μg/2 μL) or vehicle followed by 4th ventricle treatment with ODN (20 μg/2 μL), Ex-4 (0.3 μg/2 μL), or vehicle. 24-hour food intake in chow (FIG. 10A) and HFD (FIG. 10B) fed rats, kaolin intake in chow (FIG. 10C) and HFD (FIG. 10D) fed rats, and body weight change in chow (FIG. 10E) and HFD (FIG. 10F) fed rats following treatments. # indicates difference from Veh/Veh (P=0.05). All data presented as mean±SEM.

FIGS. 11A-11F. Pretreatment with an ODN antagonist attenuates peripheral Liraglutide induced hypophagia. Rats were pretreated lateral ventricle with an ODN antagonist (AntOP 100 μg/2 μL) or vehicle followed by intraperitoneal treatment with Liraglutide (50 μg/kg), or vehicle. 48 hour food intake in chow (FIG. 11A) and HFD (FIG. 11B) fed rats, kaolin intake in chow (FIG. 11C) and HFD (FIG. 11D) fed rats, and body weight change in chow (FIG. 11E) and HFD (FIG. 11F) fed rats following treatments. All data presented as mean±SEM.

FIGS. 12A-12F. 5-day combined treatment with ODN enhances peripheral Liraglutide induced hypophagia. Rats were treated 4th ventricle with ODN (100 μg/2 μL) or vehicle followed by intraperitoneal treatment with Liraglutide (25 ag/kg) or vehicle for 5 days. Daily (FIG. 12A) and cumulative (FIG. 12B) food intake, daily (FIG. 12C) and cumulative (FIG. 12D) kaolin intake, and daily body weight (FIG. 12E) in HFD fed rats following treatments. Isolated day 1 food intake (FIG. 12F). All data presented as mean±SEM.

FIGS. 13A-13F. Central tridecaneuropeptide (TDN) a cleavage product of ODN suppresses 24-hour food intake in HFD fed rats. Effect of 4th ventricle TDN (20 μg/2 μL) on 24-hour food intake in chow (FIG. 13A) and HFD (FIG. 13B) fed rats, kaolin intake in chow (FIG. 13C) and HFD (FIG. 13D) fed rats, and body weight change in chow (FIG. 13E) and HFD (FIG. 13F) fed rats following treatments. All data presented as mean±SEM.

FIGS. 14A-14F. Central TDN suppresses 24-hour food intake in rats. Effect of lateral ventricle TDN (20 μg/2 μL or 200 μg/2 VL) on 24-hour food intake in chow (FIG. 14A) and HFD (FIG. 14B) fed rats, kaolin intake in chow (FIG. 14C) and HFD (FIG. 14D) fed rats, and body weight change in chow (FIG. 14E) and HFD (FIG. 14F) fed rats following treatments. All data presented as mean±SEM.

FIGS. 15A-15C. Peripheral ODN suppresses 24-hour food intake in shrews without including emesis. Effect of intraperitoneal ODN (500 μg/kg or 5000 μg/kg) on 24-hour food intake (FIG. 15A) body weight change (FIG. 15B) and emetic episodes (FIG. 15C) following treatments. All data presented as mean±SEM.

FIGS. 16A-16B. Central ODN-based drugs suppress 24 h food intake in HFD fed rats. Effect of 4th ventricle ODN, TDN, SUODN04, and SUODN05 (20 μg/kg) on 24-hour food intake (FIG. 16A) body weight change (FIG. 16B) following treatments. All data presented as mean±SEM.

FIGS. 17A-17H. Nutritional State and GLP-1R Agonism Regulates NTS/AP DBI Protein Expression. Rats had ad libitum access to food or were 24 h fasted and received a 4th ventricle injection of vehicle of Exendin-4 (0.3 μg/2 μL) 90 minutes before sacrifice. Brains were collected and stained with a DBI antibody to detect DBI protein expression, fluorescence of which was quantified at low, moderate, and strong levels using HALO-AI software. DBI protein expression was determined in the NTS at the pre-AP level in chow (FIG. 17A) and HFD (FIG. 17B) fed rats, in the NTS at the AP level in chow (FIG. 17C) and HFD (FIG. 17D) fed rats, in the NTS at the 4th ventricle level in chow (FIG. 17E) and HFD (FIG. 17F) fed rats, and in the AP in chow (FIG. 17G) and HFD (FIG. 17H) fed rats.

FIGS. 18A-18D. Central ODN suppresses food intake more robustly in chow fed females than males. Effect of lateral ventricle ODN (20, 100, and 200 μg/2 μL) on 24 hour food intake (FIG. 18A) and body weight (FIG. 18B) in female chow fed rats, and on 24 h food intake (FIG. 18C) and body weight (FIG. 18D) in male chow fed rats following treatments. All data presented as mean±SEM.

FIGS. 19A-19D. Central ODN suppresses food intake more robustly in chow fed overnight fasted females than males. Effect of lateral ventricle ODN (20 and 100 μg/2 μL) on 24 hour food intake (FIG. 19A) and body weight (FIG. 19B) in female overnight fasted chow fed rats, and on 24 h food intake (FIG. 19C) and body weight (FIG. 19D) in male overnight fasted chow fed rats following treatments. All data presented as mean±SEM.

FIGS. 20A-20D. Central ODN suppresses food intake more robustly in HFD fed females than males. Effect of lateral ventricle ODN (20, 100, and 200 μg/2 μL) on 24 hour food intake (FIG. 20A) and body weight (FIG. 20B) in female HFD fed rats, and on 24 h food intake (FIG. 20C) and body weight (FIG. 20D) in male HFD fed rats following treatments. All data presented as mean±SEM.

FIGS. 21A-21D. Central ODN weakly suppresses food intake in HFD fed overnight fasted males. Effect of lateral ventricle ODN (20 and 100 μg/2 μL) on 24 hour food intake (FIG. 21A) and body weight (FIG. 21B) in female overnight fasted HFD fed rats, and on 24 h food intake (FIG. 21C) and body weight (FIG. 21D) in male overnight fasted HFD fed rats following treatments. All data presented as mean±SEM.

FIGS. 22A-22E. Females have higher endogenous hindbrain DBI protein expression. Brains from chow ad libitum fed male and female rats were collected and stained with a DBI antibody to detect DBI protein expression, fluorescence of which was quantified at low, moderate, and strong levels using HALO-AI software. DBI protein expression was determined in the AP (FIG. 22A), subpostrema border (FIG. 22B), NTS at the AP level (FIG. 22C), 4th ventricle border (FIG. 22D), and NTS at the post-AP level (FIG. 22E).

FIGS. 23A-23F. Females have higher endogenous relaxin-3 protein expression in nerve fibers. Brains from chow ad libitum fed male and female rats were collected and stained with a relaxin-3 (Rln3) antibody to detect Rln3 protein expression, fluorescence of which was quantified at low, moderate, and strong levels using HALO-AI software. Rln3 protein expression was determined in the central (FIG. 23A) and lateral (FIG. 23B) nucleus incertus rich with Rln3 positive neuron cell bodies, in the more caudal central (FIG. 23C) and lateral (FIG. 23D) nucleus incertus rich with Rln3 positive nerve fibers, and in the NTS at the AP level (FIG. 23E) and 4th ventricle level (FIG. 23F).

FIGS. 24A-24B. Pretreatment with ODN attenuates central Relaxin-3 induced hyperphagia. Effect of lateral ventricle ODN (20 μg/2 μL) on acute 3 h food intake (FIG. 24A) and 24 h body weight (FIG. 24B) in male HFD fed rats following treatments. All data presented as mean±SEM.

FIGS. 25A-25D. Central optimized ODN and TDN suppresses 24 h food intake in rats. ODN and TDN were optimized using an acetate salt precipitation rather than a TFA slat precipitation to maintain a neutral >4.5 pH in HEPES buffer solution. Effect of 4th ventricle ODN (200 μg/2 μL) on 24 h food intake (FIG. 25A) and body weight (FIG. 25B) in male chow fed rats, and lateral ventricle TDN (200 μg/2 μL) on 24 h food intake (FIG. 25C) and body weight (FIG. 25D) in female HFD fed rats following treatments. All data presented as mean±SEM.

FIGS. 26A-26B. Peripheral TDN suppresses 24 h food intake in overnight fasted mice. Effect of IP TDN (5 mg/kg) on 24 h food intake (FIG. 26A) and body weight (FIG. 26B) in male HFD fed overnight fasted mice following treatments. All data presented as mean±SEM.

FIGS. 27A-27D. Chronic peripheral TDN suppresses food intake in mice. Effect of daily IP TDN (5 mg/kg) on daily 24 h weight change (FIG. 27A) and food intake (FIG. 27B) and cumulative weight change (FIG. 27C) and food intake (FIG. 27D) over 9 days in male HFD fed mice following treatments. All data presented as mean±SEM.

FIGS. 28A-28C. Hindbrain ODN improves glucose tolerance in chow rats. Effect of 4th ventricle ODN (20 and 200 μg/2 μL) on glucose tolerance (FIG. 28A), glucose tolerance test area under the curve (FIG. 28B), and oral glucose stimulated plasma insulin (FIG. 28C) in chow fed rats following treatments. All data presented as mean±SEM.

FIGS. 29A-29C. Hindbrain ODN signaling regulates the counterregulatory response. 1 hour blood glucose (FIG. 29A), and 30-minute plasma corticosterone (FIG. 29B) and plasma glucagon (FIG. 29C) in chow fed rats pretreated 4th ventricle with ODN (20 μg/2 μL) or vehicle followed by 4th ventricle treatment with 5-TG (210 μg/2 μL) or vehicle. All data presented as mean±SEM.

FIGS. 30A-30D. Hindbrain ODN improves insulin tolerance in chow rats. Effect of 4th ventricle ODN (20 and 200 μg/2 μL) on insulin tolerance (FIG. 30A), glucose tolerance test area under the curve (FIG. 30B), and oral glucose stimulated plasma insulin (FIG. 30C) in chow fed rats following treatments. All data presented as mean±SEM.

FIG. 31. Pretreatment with relaxin-3 and ODN antagonist block hindbrain ODN improvements in insulin tolerance in chow rats. Insulin tolerance test in chow fed rats pretreated 4th ventricle with relaxin-3 (Rln3 0.5 μg/2 μL) or AntOP (100 μg/2 μL) or vehicle followed by 4^{th} ventricle treatment with ODN (200 μg/2 μL) or vehicle. All data presented as mean±SEM

FIGS. 32A-32B. (FIG. 32A) DLS (Zetasizer Ultra, Malvern Panalytical) scans of TDN at concentrations from 10 nM to 10 mM in 50 mM HEPES buffer pH 7.4. (FIG. 32B) Agonism of Relaxin-3 receptor with agonist relaxin-3-B-chain peptide (RLN3b).

FIG. 33. ⁸⁹Zirconium labelled TDN PET scan in athymic nude mice (n=3) in coronal view demonstrating robust brain uptake. Insulin tolerance TDN-DFO (0.19 mg, 80 nmol) was reconstituted in saline (pH 7) and reacted with ⁸⁹Zr (930 mCi, 34.4 MBq) in a 1:5 ratio (peptide: activity) for 45 minutes at RT. Radiochemical purity was assessed via instant thin-layer silica gel chromatography (iTLC-SG; Agilent Technologies) using 50 mM EDTA as the mobile phase. The reaction was quenched by adding EDTA (5 mL, 50 mM) to sequester unreacted activity, and the unbound radiometal was removed by centrifugation via spin column (MWCO=10 kDa) with sterile saline as the solvent. Athymic nude mice (n=3) were injected (i.v.) with [89Zr]-DFO-TDN in sterile saline (4.8-5.6 MBq, 130-150 μCi, 60-70 nmol) in the lateral tail vein. Small-animal PET was conducted 48 hours post-injection using a Bruker Albira Si PET/CT while the mice were anesthetized with 2% isoflurane. Images were reconstructed through maximum likelihood expectation maximization in 12 iterations and 0.75 mm voxel resolution and were analyzed using PMOD version 4.3 software. Volumes of interest were measured manually by drawing on the target site across various planar sections and expressed as percent injected dose per volume of tissue (% ID/mL).

### DETAILED DESCRIPTION

Endozepines are a family of astroglia-secreted proteins including the diazepam binding inhibitor (DBI) and its processing products, which have been originally isolated and characterized as endogenous ligands of benzodiazepine receptors. It is now clearly established that the octadecaneuropeptide ODN, acting through the central-type benzodiazepine receptor or an orphan metabotropic receptor, exerts important functions such as pro-conflict behavior, induction of anxiety, inhibition of pentobarbital-provoked sleep, decrease of water consumption and reduction of food intake. To mediate its effects, ODN regulates both glial cell and neuronal activities by acting on neurosteroid biosynthesis and/or neuropeptide expression. In addition, ODN stimulates astrocyte proliferation and protects both neurons and astrocytes from oxidative stress-induced cell death. The antiapoptotic effect of ODN on neural cells is mediated through activation of the ODN metabotropic receptor positively coupled to PKA, PKC and MAPK/ERK transduction pathways, which ultimately reduces the pro-apoptotic gene Bax and stimulates Bel-2 expression, thereby inhibiting intracellular reactive oxygen species accumulation. The imbalance in favor of Bcl2 promotes mitochondria functions and blocks in turn caspases activation while at the same time, ODN also activates the endogenous antioxidant system i.e., glutathione biosynthesis, and expression and activities of antioxidant enzymes. In cultured astrocytes, DBI expression is upregulated during moderate oxidative stress, and authentic ODN production is increased, suggesting that ODN may act as a paracrine factor protecting neighboring neurons.

Obesity alters multiple aspects of hindbrain ODN signaling, including a longer time course of ODN hypophagia, including possible difference in diazepam binding inhibitor (DBI) cleavage to ODN (recombinant DBI protein is hypophagic in chow but not high-fat diet (HFD) rats), the ODN antagonist AntOP is hypophagic in HFD but not chow rats providing a possible new site of action and/or modified target interactions.

ODN signaling may be downstream of, and partially mediate, the effects of glucagon-like peptide-1 receptor (GLP-1R) signaling, and thus partially mediates all existing GLP-1-based pharmacotherapies currently used to treat diabetes and obesity. Nonetheless, ODN and novel peptide derivatives (e.g., TDN) thereof signaling appears to not be maxed out by GLP-IR agonism as ODN and GLP-1R agonist co-treatment have additive hypophagia effects.

Antagonizing ODN signaling with either an antibody targeted against DBI or a peptide antagonist of the ODN receptor (AntOP), attenuates the hypophagic and body weight effects of central and peripheral GLP-1R agonists. Because nutritional state regulates hindbrain DBI protein expression, GLP-IR signaling may contribute to this effect. ODN may facilitate transport of GLP-1R agonists across the blood brain barrier, specifically at the tanycyte borders, both across the 4th ventricle and the sub-postrema border, to regulate brain penetrance of GLP-1R agonists. Multiple cleavage products of ODN are physiologically active and suppress food intake, allowing for the creation of new forms of non-naturally occurring optimized peptides.

In the present studies, the ODN receptor has been deorphanized. We have discovered that ODN is an endogenous antagonist of the relaxin-3 receptor (RXFP3) and novel peptide derivatives of ODN (e.g., TDN; tridecaneuropeptide; SEQ ID NO: 3) also antagonize the relaxin-3 receptor. Relaxin-3 is an orexigenic neuropeptide that promotes weight gain and becomes upregulated in obesity to defend against weight loss. Thus, antagonizing this signal to preserve a lower body weight may powerfully help overcome the typically observed weight loss plateau seen with current anti-obesity pharmacotherapies.

### Definitions

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

In this invention, “a”, “or” and “an” can mean “at least one,” or “one or more”, etc., unless clearly indicated otherwise by context. The term “or” means “and/or” unless stated otherwise. In the case of a multiple-dependent claim, however, use of the term “or” refers back to more than one preceding claim in the alternative only.

Furthermore, a compound “selected from the group consisting of” refers to one or more of the compounds in the list that follows, including mixtures (i.e., combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The terms “agent” and “test compound” denote a chemical compound, a mixture of chemical compounds, a biological macromolecule, or an extract made from biological materials such as bacteria, plants, fungi, or animal (particularly mammalian) cells or tissues. Biological macromolecules include peptides, peptide/DNA complexes, siRNA, shRNA, antisense oligonucleotides, and any nucleic acid-based molecule which encoded the proteins described herein.

It is also contemplated that the term “compound” or “compounds” refers to the compounds discussed herein and includes precursors and derivatives of the compounds, and pharmaceutically acceptable salts of the compounds, precursors, and derivatives.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

A “derivative” of a polypeptide, polynucleotide or fragments thereof means a sequence modified by varying the sequence of the construct, e.g., by manipulation of the nucleic acid encoding the protein or by altering the protein itself. “Derivatives” of a polypeptide sequence refers to any isolated amino acid molecule that contains significant sequence similarity to the peptide sequence or a part thereof. In addition, “derivatives” include such isolated nucleic acids containing modified nucleotides or mimetics of naturally occurring nucleotides encoding the derivative peptides.

The term “functional” as used herein implies that the nucleic or amino acid sequence is functional for the recited assay or purpose.

For purposes of the invention, “nucleic acid”, “nucleotide sequence” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated. “Isolated” is not meant to exclude artificial or synthetic mixtures with other compounds or materials, or the presence of impurities that do not interfere with the fundamental activity, and that may be present, for example, due to incomplete purification. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

A “specific binding pair” comprises a specific binding member (sbm) and a binding partner (bp) which have a particular specificity for each other and which in normal conditions bind to each other in preference to other molecules. Examples of specific binding pairs are antigens and antibodies, biotin and streptavidin, ligands and receptors and complementary nucleotide sequences. The skilled person is aware of many other examples. Further, the term “specific binding pair” is also applicable where either, or both, of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

According to the present invention, an isolated or biologically pure molecule or cell is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The term “delivery” as used herein refers to the introduction of foreign molecule (i.e., miRNA encoding the polypeptide of interest) into cells. The term “administration” as used herein means the introduction of a foreign molecule into the body or a cell. The term is intended to be synonymous with the term “delivery”.

### Peptides

The peptides of the invention inhibit or modulate ODN activity. The terms “inhibition” or “inhibit” refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, “reduce” or “inhibit” refers to the ability to cause an overall decrease of 20% or greater. In another embodiment, “reduce” or “inhibit” refers to the ability to cause an overall decrease of 50% or greater. In yet another embodiment, “reduce” or “inhibit” refers to the ability to cause an overall decrease of 75%, 85%, 90%, 95%, or greater.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

The term “inhibitor” refers to an agent that slows down or prevents a particular chemical reaction, signaling pathway or other process, or that reduces the activity of a particular reactant, compound, or enzyme.

The term “octadecaneuropeptide” or “ODN” and its precursor diazepam-binding inhibitor (DBI) are peptides belonging to the family of endozepines. Endozepines are exclusively produced by astroglial cells in the central nervous system of mammals, and their release is regulated by stress signals and neuroactive compounds. There is now compelling evidence that ODN protects cultured neurons and astrocytes from apoptotic cell death induced by various neurotoxic agents.

The phrase “ODN antagonist” refers to a class of agents that inhibit the action of ODN. The phrase “ODN agonist” refers to a class of agents that potentiate or enhance ODN activity. The peptides of interest herein include naturally occurring ODN and derivatives thereof, amongst others, and are provided in Table 1.

TABLE 1
ODN and ODN based peptide sequences. Peptides ODN, AntOP, TDN, OP and SUODN-03,
-04, and -05 have been synthesized and assayed. Gln(alkyn), Ala(Alkyn), Gly(Alkyn) and
Lys(N3) are modified amino acids for bioconjugation including lipidation and/or
fluorescent tagging*.

ODN: Gln-Ala-Thr-Val-Gly-Asp-Val-Asn-Thr-Asp-Arg-Pro-Gly-Leu-Leu-Asp-Leu-Lys
AntOP: Arg-Pro-Gly-Leu-(D-Leu)-Asp-Leu-Lys
TDN (SUODN-01): Asp-Val-Asn-Thr-Asp-Arg-Pro-Gly-Leu-Leu-Asp-Leu-Lys
OP (SUODN-02): Arg-Pro-Gly-Leu-Leu-Asp-Leu-Lys
SUODN-03: Arg-Pro-Gly-Leu-Leu
SUODN-04: Asp-Val-Asn-Thr-Asp-Arg-Pro-Gly
SUODN-05: Asp-Val-Asn-Thr-Asp-Arg-Pro-Gly-Leu-Leu
SUODN-06: Gln-Ala-Thr-Val-Gly-Asp-Val-Asn-Thr-Asp-Arg-Pro-Gly
SUODN-07: Gln-Ala-Thr-Val-Gly-Asp-Val-Asn-Thr-Asp-Arg-Pro-Gly-Leu-Leu
SUODN-08: Gln(alkyn)-Ala-Thr-Val-Gly-Asp-Val-Asn-Thr-Asp-Arg-Pro-Gly-Leu-Leu-Asp-
Leu-Lys(N3)
SUODN-09: Gln(alkyn)-Ala-Thr-Val-Gly-Asp-Val-Asn-Thr-Asp-Arg-Pro-Gly-Lys(N3)-Leu-
Asp-Leu-Lys
SUODN-10: Gln(alkyn)-Ala-Thr-Val-Gly-Asp-Val-Asn-Thr-Asp-Arg-Pro-Gly-Leu-Lys(N3)-
Asp-Leu-Lys
SUODN-11: Gln(alkyn)-Ala-Thr-Val-Gly-Asp-Val-Asn-Thr-Asp-Arg-Pro-Gly-Leu-Leu-Asp-
Lys(N3)-Lys
SUODN-12: Gln-Ala(alkyn)-Thr-Val-Gly-Asp-Val-Asn-Thr-Asp-Arg-Pro-Gly-Leu-Leu-Asp-
Leu-Lys(N3)
SUODN-13: Gln-Ala(alkyn)-Thr-Val-Gly-Asp-Val-Asn-Thr-Asp-Arg-Pro-Gly-Lys(N3)-Leu-
Asp-Leu-Lys
SUODN-14: Gln-Ala(alkyn)-Thr-Val-Gly-Asp-Val-Asn-Thr-Asp-Arg-Pro-Gly-Leu-Lys(N3)-
Asp-Leu-Lys
SUODN-15: Gln-Ala(alkyn)-Thr-Val-Gly-Asp-Val-Asn-Thr-Asp-Arg-Pro-Gly-Leu-Leu-Asp-
Lys(N3)-Lys
SUODN-16: Gln-Ala-Thr-Val-Gly(alkyn)-Asp-Val-Asn-Thr-Asp-Arg-Pro-Gly-Leu-Leu-Asp-
Leu-Lys(N3)
SUODN-17: Gln-Ala-Thr-Val-Gly(alkyn)-Asp-Val-Asn-Thr-Asp-Arg-Pro-Gly- Lys(N3)-Leu-
Asp-Leu-Lys
SUODN-18: Gln-Ala-Thr-Val-Gly(alkyn)-Asp-Val-Asn-Thr-Asp-Arg-Pro-Gly-Leu-Lys(N3)-
Asp-Leu-Lys
SUODN-19: Gln-Ala-Thr-Val-Gly(alkyn)-Asp-Val-Asn-Thr-Asp-Arg-Pro-Gly-Leu-Leu-Asp-
Lys(N3)-Lys
*SEQ ID NOS; 1-21 are shown in descending order.

In certain embodiments, the residues of the protein or peptide are sequential, without any non-genetically encoded amino acids, or synthetic amino acids interrupting the sequence of amino acid residues. The peptides can be modified as described above. In other embodiments, the sequence may comprise one or more non-genetically encoded or synthetic amino acid moieties. In particular embodiments, the sequence of residues of the peptide may be interrupted by one or more non-genetically encoded or synthetic amino acid moieties, including but not limited to those shown in Table 2.

TABLE 2
Non-genetically Encoded or Synthetic Amino Acids

| Abbreviation | Amino Acid | Abbreviation | Amino Acid |
|---|---|---|---|
| AcLys | N-Acetyl lysine | 4FPhe | 4-Fluorophenylalanine |
| Aad | 2-Aminoadipic acid | Hip | Hippuric acid |
| Baad | 3-Aminoadipic acid | hArg | Homoarginine |
| Bala | β-alanine, 3-Amino- | hCys | Homocysteine |
| | propionic acid | | |
| Abu | 2-Aminobutyric acid | hSer | Homoserine |
| 4Abu | 4-Aminobutyric acid, | AHyl | allo-Hydroxylysine |
| | piperidinic acid | | |
| Aha | 6-Aminohexaboic acid | 3.Hyp | 3-Hydroxyproline |
| Ahe | 2-Aminoheptanoic acid | 4Hyp | 4-Hydroxyproline |
| Aib | 2-Aminoisobutyric acid | Hyl | Hydroxylysine |
| Baib | 3-Aminoisobutyric acid | Ide | Isodesmosine |
| Apa | 5-Aminopentoic acid | AIle | allo-Isoleucine |
| pAmPhe | p-Aminophenylalanine | MSO | Methionine sulfoxide |
| Apm | 2-Aminopimclic acid | McAla | 2-Methyl-alanine |
| Dap | 3-Aminopropionic acid | MeGly | N-Methylglycine, sarcosine |
| BztAla, B | 3-Benzothiazol-2-yl-alanine | MeIle | N-Methylisoleucine |
| BzPhe | para-Benzoyl-phenylalanine | MeLys | 6-N-Methyllysine |
| tBuA | t-Butylalanine | MeVal | N-Methylvaline |
| tBuG | t-Butylglycine | Nal | 2-Naphthylalanine |
| cGlu | gamma-Carboxyglutamate | Nwa | Norvaline |
| Cit | Citrulline | Nle, J | Norleucine |
| 4ClPhe | 4-Chlorophenylalanine | Oct | Octahydro-1H-indole-2- |
| | | | carboxylic acid |
| Cha | Cyclohexylalanine | Orn | Ornithine |
| Dab | 2,3-Diaminobutyric acid | Pen | Penicillamine |
| Dbu | 2,4-Diaminobutyric acid | Phg | 2-Phenylglycine |
| Des | Desmosine | pSer | Phosphoserine |
| Dpm | 2,2'-Diaminopimelic acid | pThr | Phosphothreonine |
| Dpr, Z | 2,3-Diaminopropionic acid | pTyr | Phosphotyrosine |
| EtAsn | H-Ethylasparagine | PropGly | Propargylglycine |
| EtGly | N-Ethylglycine | Sta | Statine |
| 2FPhe | 2-Fluorophenylalanine | Tic | 1,2,3,4-Tetrahydroisoquinoline- |
| | | | 3-carboxylic acid |
| 3FPhe | 3-Fluorophenylalanine | Thi | beta-2-Thienylalanine |

TABLE 3
Structural modification strategies to optimize peptide
function and/or stability and/or formulation/solubility.
AA = amino acid. Fc = ‘Fragment crystallizable’ of IgG.

| General Stability/ | |
|---|---|
| Clearance Approach | Examples |
| N-/C- termini protection | N-acetylation and C-amidation |
| Use of D-isomer and/ | Replacement of AAs at sites of proteolysis |
| or non-canonical AA | |
| Conjugation to | Lipidation e.g. (C15-C20 carboxylic acid |
| macromolecules | capped) at C- or N-termini and/or sites that |
| | do not remove function as screened in vitro. |
| Fusion technology | Recombinant Fc-Peptide, CPP, targeting tag |
| | (e.g., ligand for the transferrin receptor) |

These modifications will produce analogs with prolonged half-lives allowing, for example, once weekly dosing.

In certain embodiments, the present invention includes peptides that have at least 80% identity to anyone of the peptides described herein. In certain embodiments, the peptides of the invention have a sequence identity of at least 90% identity, at least 91% identity, at least 92% identity, at least 93% identity, at least 94% identity, at least 95% identity, at least 96% identity, at least 97% identity, at least 98% identity, at least 99% identity, or 100% identity.

Preferably, the peptides of the above-described sequences and functional equivalents thereof which act to modulate obesity upon administration. As used herein, the term “functional equivalent” is intended to include amino acid sequence variants having amino acid substitutions in some or all the proteins, or amino acid additions or deletions in some of the proteins. The amino acid substitutions arc preferably conservative substitutions. Examples of the conservative substitutions of naturally occurring amino acids are as follow: aliphatic amino acids (Gly. Ala, and Pro), hydrophobic amino acids (Ile, Leu, and Val), aromatic amino acids (Phe, Tyr, and Trp), acidic amino acids (Asp, and Glu), basic amino acids (His, Lys, Arg, Gln, and Asn), and sulfur-containing amino acids (Cys, and Met). The deletions of amino acids are preferably located in a region which is not directly involved in the activity of the peptide.

In the present context, the term “variant” refers to a nucleic acid sequence or polypeptide comprising a sequence, which differs (by deletion, insertion, and/or substitution of a nucleic acid or amino acid including non-naturally occurring amino acid) in one or more nucleic acid or amino acid positions from that of a wild type nucleic acid or polypeptide sequence.

In the present context, the term “linker” refers to a connection between two protein coding sequences or their protein products. Linkers comprise a stretch of contiguous nucleic acids or amino acids, which holds at least one cleavage site that enables separation of the genes or their products through cleavage of the linker. Preferably, the linker comprises a cleavage site at its 5′ end and a cleavage site at its 3′ end, or a cleavage site at its N-terminal end and a cleavage site at its C-terminal end.

The peptide may be fused to biotin, Poly-lysine, lysozyme, Green fluorescent protein (and derivatives), SUMO or other desired proteinaceous tags for attachment to electrodes, nanotubes or desired surfaces (e.g. electro-transducing, mineral), as well as any protein interaction partner desired to be investigated Production of the desired peptide sequence can be carried out in E. coli using existing technologies, e.g. with protein fusion tags that can either be removed or left as desired. In certain embodiments, the peptide of interest may be fused via a linker.

The peptide can be fused to one or more cell penetrating peptides (CPP) which are useful for facilitating delivery of the peptide into target cells. CPPs are known to the skilled person and include without limitation, penetratin (RQIKIWFQNRRMKWKK) (SEQ ID NO: 22), VP22 peptide (DAATATRGRSAASRPTER PRAPARSASRPRRVD) (SEQ ID NO: 23), MAP (KLALKLALKALKAALKLA-amide) (SEQ ID NO: 24), Transportin (GWTLNSAGYLLGKINLKALAALAKKIL-amide) (SEQ ID NO: 25) R7 (RRRRRRR) (SEQ ID NO: 26), MPG (GALFLGWLGAAGSTMGAPKKRKV) (SEQ ID NO: 27), and Pep-1 (KETWWETWWTEWSQPKKKRKV) (SEQ ID NO: 28) and tat (YGRKKRRQRRR; SEQ ID NO: 29).

The peptide can be expressed as a fusion to larger proteins, facilitating expression at large scales, ease of purification, and ensuring quality of product. Expression systems can also be leveraged to generate large sequence libraries, allowing for directed evolution for targeted properties. Fusion also allows the peptide to be incorporated into other proteins useful for the treatment of obesity or other metabolic disorders.

As noted above, the invention also includes polynucleotides encoding the peptides or fusion proteins comprising the peptide described herein. Those of skill in the art understand the degeneracy of the genetic code and that a variety of polynucleotides can encode the same polypeptide. In some embodiments, the polynucleotides (i.e., polynucleotides encoding the fusion polypeptides) may be codon-optimized for expression in a particular cell including, without limitation, a plant cell, bacterial cell, or algal cell. Any polynucleotide sequences may be used which encode a desired form of the polypeptides described herein. The polynucleotide sequences which encode the polypeptides of the invention represent non-naturally occurring sequences. Computer programs for generating degenerate coding sequences are available and can be used for this purpose. Pencil, paper, the genetic code, and a human hand can also be used to generate degenerate coding sequences.

In the present context, the term “codon optimization” refers to changing the codons of a nucleotide sequence without altering the amino acid sequence that it encodes in order to favor expression in a specific species. Codon optimization may be used to increase the abundance of the peptide or protein that the nucleotide sequence encodes since “rare” codons are removed and replaced with abundant codons.

Regarding the fusion polypeptides disclosed herein, the phrases “% sequence identity,” “percent identity,” or “% identity” refer to the percentage of residue matches between at least two amino acid sequences aligned using a standardized algorithm. Methods of amino acid sequence alignment are well-known. Some alignment methods consider conservative amino acid substitutions. Such conservative substitutions generally preserve the charge and hydrophobicity at the site of substitution, thus preserving the structure (and therefore function) of the polypeptide. Percent identity for amino acid sequences may be determined as understood in the art. The structural similarity is typically at least 90% identity, at least 91% identity, at least 92% identity, at least 93% identity, at least 94% identity, at least 95% identity, at least 96% identity, at least 97% identity, at least 98% identity, or at least 99% identity.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30. at least 40, at least 50, at least 70 or at least 150 contiguous residues. Such lengths are exemplary only, and it is understood that any fragment length supported by the sequences shown herein, may be used to describe a length over which percentage identity may be measured.

### In Vitro Synthesis of Peptides

Peptides can be synthesized chemically either in solution or on a solid phase. The process involves directed and selective formation of an amide bond between an N-protected amino acid and an amino acid bearing a free amino group and protected carboxylic acid. In solid phase synthesis, the carboxyl protecting group is linked to a polymer support. Following bond formation, the amino-protecting group of the dipeptide is removed, and the next N-protected amino-acid is coupled.

Solid-phase peptide synthesis (SSPS) is the most frequently used method of peptide synthesis due to its efficiency, simplicity, speed, and ease of parallelization. SPPS involves sequential addition of amino and side-chain protected amino acid residues to an amino acid or peptide attached to an insoluble polymeric support. Either an acid-labile Boc group (Boc SPPS) or base-labile Fmoc-group (Fmoc SPPS) is used for N-ca-protection. After removal of this protecting group, the next protected amino acid is added using either a coupling reagent or pre-activated protected amino acid derivative. The C-terminal amino acid is anchored to the resin via a linker, the nature of which determines the conditions required to release the peptide from the support after chain extension. Sidechain protecting groups are often chosen so as to be cleaved simultaneously with detachment of the peptide from the resin.

Peptides of 50 amino acids can be routinely prepared although the synthesis of proteins of over 100 amino acids are commonly reported. Longer proteins can be made by native chemical ligation of fully deprotected peptides in solution. With this method, it is possible to synthesize natural peptides that are difficult to express in bacteria, to incorporate unnatural or D-amino acids, and to generate cyclic, branched, labelled, and post-translations modified petides.

Liquid-phase peptide synthesis, usually utilizing Boc or Z-amino protection, has been superseded by solid-phase peptide synthesis except for existing processes of large-scale synthesis of peptides for industrial purposes. Desired sequences can be developed by any one of the several commercial entities who provide this service for a fee, including Sigma Aldrich, and Avivasysbio for example.

### Vectors and Production

Transgenic cells expression said polynucleotides also form an aspect of the invention. A transgenic cell may be obtained by introducing a recombinant nucleic acid molecule that encodes a protein of this disclosure. As used herein, the term “recombinant nucleic acid” refers to a polynucleotide that is manipulated by human intervention. A recombinant nucleic acid molecule can contain two or more nucleotide sequences that are linked in a manner such that the product is not found in a cell in nature. In particular, the two or more nucleotide sequences can be operatively linked and, for example, can encode a fusion polypeptide. A recombinant nucleic acid molecule also can be based on, but manipulated so as to be different, from a naturally occurring polynucleotide, for example, a polynucleotide having one or more nucleotide changes such that a first codon, which normally is found in the polynucleotide, is biased for chloroplast codon usage, or such that a sequence of interest is introduced into the polynucleotide, for example, a restriction endonuclease recognition site or a splice site, a promoter, a DNA origin of replication, or the like.

Any appropriate technique for introducing recombinant nucleic acid molecules into cells may be used. Techniques for nuclear and chloroplast transformation are known and include, without limitation, electroporation, biolistic transformation (also referred to as micro-projectile/particle bombardment), agitation in the presence of glass beads, and Agrobacterium-based transformation.

As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as integrating vectors.

The constructs and vectors provided herein may be prepared by methods available to those of skill in the art. Notably each of the constructs or expression cassettes claimed are recombinant molecules and as such do not occur in nature. Generally, the nomenclature used herein, and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The constructs and expression cassettes provided herein may include a promoter operably linked to any one of the polynucleotides described herein but need not have a promoter and may be used for homologous recombination into the cell. Alternatively, the constructs may include a promoter and the promoter may be a heterologous promoter or an endogenous promoter associated with the polypeptide.

As used herein, the terms “heterologous promoter,” “promoter,” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the disclosed polynucleotides are operably connected to the promoter. As used herein, a polynucleotide is “operably connected” or “operably linked” when it is placed into a functional relationship with a second polynucleotide sequence. For instance, a promoter is operably linked to a polynucleotide if the promoter is connected to the polynucleotide such that it may affect transcription of the polynucleotides. In various embodiments, the polynucleotides may be operably linked to at least 1, at least 2, at least 3, at least 4, at least 5, or at least 10 promoters.

Heterologous promoters useful in the practice of the present invention include, but are not limited to, constitutive, inducible, temporally regulated, developmentally regulated, chemically regulated, tissue-preferred and tissue-specific promoters. The heterologous promoter may be a plant, animal, bacterial, fungal, or synthetic promoter.

### Methods of Treatment and Administration

The methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “reducing” or “inhibiting” as used herein refers to administering a compound prior to, or during the onset of clinical symptoms of a disease or conditions so as to reduce a physical manifestation of aberrations associated with the disease or condition.

The term “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The terms “treat”, “treating”, and “treatment” as used herein, refer to eliciting the desired biological response, such as a therapeutic and prophylactic effect, respectively. In some embodiments, a therapeutic effect comprises one or more of a decrease/reduction in obesity, a decrease/reduction in the severity of obesity (such as, for example, a reduction or inhibition of development or obesity), a decrease/reduction in symptoms and obesity-related effects, delaying the onset of symptoms and obesity-related effects, reducing the severity of symptoms of obesity-related effects, reducing the severity of an acute episode, reducing the number of symptoms and obesity-related effects, reducing the latency of symptoms and obesity-related effects, an amelioration of symptoms and obesity-related effects, reducing secondary symptoms, reducing secondary infections, preventing relapse to obesity, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of obesity development/progression (such as, for example, a complete or partial avoidance/inhibition or a delay), or an increased survival time of the affected host animal, following administration of a therapeutic protocol. Treatment of obesity encompasses the treatment of subjects already diagnosed as having any form of obesity at any clinical stage or manifestation, the delay of the onset or evolution or aggravation or deterioration of the symptoms or signs of obesity, and/or preventing and/or reducing the severity of obesity.

In another aspect, provided herein are methods for treating a patient comprising administration of the peptides of interest. In any of the embodiments described herein, the subject can be obese, or have excessive weight, elevated BMI, elevated body fat mass, percentage, or volume, and/or excessive food intake. In any of the embodiments described herein, the subject can be obese. In any of the embodiments described herein, the subject can have excessive weight. In any of the embodiments described herein, the subject can have elevated BMI. In any of the embodiments described herein, the subject can have elevated body fat mass, percentage, or volume. In any of the embodiments described herein, the subject can have excessive food intake.

Symptoms of obesity include, but are not limited to, excess body fat accumulation (particularly around the waist), breathlessness, increased sweating, snoring, inability to cope with sudden physical activity, feeling very tired every day, back and joint pains, skin problems (from moisture accumulating in the folds of skin).

Methods of treating a subject having obesity, the methods comprising administering a ODN peptide or functional derivative thereof to the subject are provided. Also disclosed are methods of treating a subject having excessive weight, the methods comprising administering a ODN peptide to the subject. The present disclosure also provides methods of treating a subject having elevated BMI, the methods comprising administering an effective amount of ODN peptide to the subject. Methods of treating a subject having elevated body fat mass, percentage, or volume, the methods comprising administering an ODN peptide to the subject are also described. Finally, treatment of a subject having excessive food intake, comprising administering an ODN peptide to the subject are also disclosed.

The present disclosure also provides methods of treating a subject to prevent weight gain or to maintain weight loss, the method comprising administering an effective amount of ODN peptide to the subject.

In certain embodiments, methods of treating a metabolic disease or disorder in a subject, comprising administering an effective amount of ODN peptide to the subject are provided.

As used herein, the term “metabolic disease” or “metabolic disorder” is also called a metabolic syndrome and refers to a set of abnormal states such as an increase in body fat, an increase in blood pressure, an increase in blood sugar, and abnormal lipids in blood, which increase the risk of cerebral cardiovascular diseases and diabetes mellitus. The metabolic disease is not a single disease, but a comprehensive disease caused by genetic predisposition and environmental factors, and in the present invention, may be selected from the group consisting of obesity, diabetes mellitus, dyslipidemia, insulin resistance, hepatic steatosis, hypercholesterolemia, and non-alcoholic fatty liver, and may be more preferably obesity or diabetes mellitus, but is not limited thereto.

As used herein, the term “diabetes mellitus”, as a type of metabolic disease such as an insufficient amount of insulin secreted or absence of normal function, is characterized by high blood sugar with high blood glucose concentration and causes various symptoms and signs due to hyperglycemia and releases glucose from urine. Diabetes mellitus includes type 1 diabetes mellitus which occurs when insulin is not secreted largely due to the destruction of pancreatic beta cells, and type 2 diabetes mellitus caused by insufficient insulin secretion in the body or insulin resistance in which cells do not respond to insulin. In the present invention, diabetes mellitus includes both type 1 diabetes mellitus and type 2 diabetes mellitus. In certain embodiments, the method further comprises administering a second therapeutic agent that treats or inhibits obesity. Nonlimiting examples of therapeutic agents that treat or inhibit obesity and/or increased BMI include, but are not limited to, GLP1R agonists, melanocortin 4 receptor (MC4R) agonists, sibutramine, orlistat, phentermine, lorcaserin, naltrexone, liraglutide, diethylpropion, bupropion, metformin, pramlintide, topiramate, and zonisamide, or any combination thereof.

Administration of the therapeutic agents that treat or inhibit obesity and/or ODN peptide modulators can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period such as, for example, 6 months, 1 year, or more.

Administration of the therapeutic agents that treat or inhibit obesity and/or ODN peptides can occur by any suitable route including, but not limited to, parenteral, intravenous, oral, buccal, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

A non-limiting example of an effective dose range for a therapeutic compound described herein is from about 0.1 and 5,000 mg/kg of body weight/per day. One of ordinary skill in the art would be able to study the relevant factors and make the determination regarding the effective amount of the therapeutic compound without undue experimentation.

The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. See, e.g., Remington's Pharmaceutical Sciences, latest edition, by E. W. Martin Mack Pub. Co., Easton, PA, which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In one aspect, humans, and non-humans, including solutions such as sterile water, saline, and buffered solutions at physiological pH. Other compounds will be administered according to standard procedures used by those skilled in the art.

These compositions can take the form of solutions, suspensions, emulsion, tablets, pills, capsules, powders, sustained-release formulations and the like.

In some embodiments, the therapeutic agents that treat or inhibit obesity and/or ODN peptide modulators (such as any of the peptide ligands disclosed herein) are administered intrathecally (i.e., introduction into the subarachnoid space of the spinal cord or into the spinal canal so that the therapeutic agent can reach the cerebrospinal fluid of a subject, or introduction into the anatomic space or potential space inside a sheath, including, by way of non-limiting examples, the arachnoid membrane of the brain or spinal cord). In some embodiments, intrathecal administration results in the therapeutic agent acting on, without limitation, the cortex, the cerebellum, the striatum, the cervical spine, the lumbar spine, or the thoracic spine. Therapeutic agents administered intrathecally may ultimately act on targets throughout the entire central nervous system. In some embodiments, the intrathecal administration is into the cisterna magna or by the lumbar area or region. In some embodiments, the intrathecal administration into the lumbar area or region results in delivery of the therapeutic agent to the distal spinal canal.

Exemplary methods for intrathecal administration are described in, for example, Lazorthes et al., Advances in Drug Delivery Systems and Applications in Neurosurgery, 143-192. In some embodiments, the intrathecal administration is by injection, by bolus injection, by a catheter, or by a pump. In some embodiments, the intrathecal administration is by lumber puncture. In some embodiments, the pump is an osmotic pump. In some embodiments, the pump is implanted into subarachnoid space of the spinal canal, below the skin of the abdomen, or behind the chest wall. In some embodiments, the intrathecal administration is by an intrathecal delivery system for a therapeutic substance including a reservoir containing a volume of the therapeutic agent and a pump configured to deliver at least a portion of the therapeutic substance contained in the reservoir. In some embodiments, intrathecal administration is through intermittent or continuous access to an implanted intrathecal drug delivery device (IDDD). In some embodiments, the therapeutic substance is an inhibitory nucleic acid molecule. In some embodiments, the amount of the nucleic acid molecule administered intrathecally ranges from about 10 μg to about 2 mg, from about 50 μg to about 1500 μg, or from about 100 μg to about 1000 μg. In some embodiments, the therapeutic agent is disposed within a pharmaceutical composition. In some embodiments, the pharmaceutical composition does not comprise a preservative.

### Parenteral Formulations

The peptides described herein can be formulated for parenteral administration. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

If for intravenous administration, the compositions are packaged in solutions of sterile isotonic aqueous buffer. Where necessary, the composition may also include a solubilizing agent. The components of the composition are supplied either separately or mixed in unit dosage form, for example, as a dry lyophilized powder or concentrated solution in a hermetically sealed container such as an ampoule or sachet indicating the amount of active agent. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, using a coating, such as lecithin, by the maintenance of the required particle size in the case of dispersion and/or using surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine, and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

The parenteral formulations described herein can be formulated for controlled release including immediate release, delayed release, extended release, pulsatile release, and combinations thereof.

### Nano- and Microparticles

For parenteral administration, the one or more compounds, and optional one or more additional active agents, can be incorporated into microparticles, nanoparticles, or combinations thereof that provide controlled release of the compounds and/or one or more additional active agents. In forms wherein the formulations contain two or more peptides, the peptides can be formulated for the same type of controlled release (e.g., delayed, extended, immediate, or pulsatile) or the peptides can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the peptide(s). Release of the peptide(s) is controlled by diffusion of the protein(s) out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for protein containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the protein(s) can be incorporated into microparticles prepared from materials which are insoluble in aqueous solution or slowly soluble in aqueous solution but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of protein containing microparticles. Additionally, proteins, polysaccharides, and combinations thereof, which are water-soluble, can be formulated with peptide into microparticles and subsequently cross-linked to form an insoluble network. For example, cyclodextrins can be complexed with individual drug molecules and subsequently cross-linked.

Method of Making Nano- and Microparticles Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some forms, drug in a particulate form is homogeneously dispersed in a water-insoluble or slowly water-soluble material. To minimize the size of the drug particles within the composition, the drug powder itself may be milled to generate fine particles prior to formulation. The process of jet milling, known in the pharmaceutical art, can be used for this purpose. In some forms, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

### Injectable/Implantable Formulations

The compounds described herein can be incorporated into injectable/implantable solid or semi-solid implants, such as polymeric implants. In some forms, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication requires polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent.

Alternatively, the compounds can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

The release of the one or more compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

### Enteral/Oral/Lingual Formulations

Oral formulations can include standard carriers such as pharmaceutical grades of mannitol, lactose, sodium saccharine, starch, magnesium stearate, cellulose, magnesium carbonate, etc. Such compositions will contain a therapeutically effective amount of the compound and/or antibiotic together with a suitable amount of carrier so as to provide the proper form to the patient based on the mode of administration to be used.

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition, which may include plasticizers, pigments, colorants, stabilizing agents, and glidants.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes. natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the drug and a controlled release polymer or matrix. Alternatively, the drug particles can be coated with one or more controlled release coatings prior to incorporation into the finished dosage form.

In another form, the one or more compounds and optional one or more additional active agents are in dispensed form, the one or more compounds, and optional one or more additional active agents are formulated into a sold oral dosage form, such as a tablet or capsule, and the solid dosage form is coated with one or more controlled release coatings, such as a delayed release coatings or extended-release coatings. The coating or coatings may also contain the compounds and/or additional active agents.

The materials and methods below are provided to facilitate the practice of the present invention.

### Animals

Adult male Sprague-Dawley rats (Charles River) were individually housed under a 12 h-light: 12 h-dark cycle in a temperature and humidity-controlled satellite vivarium and had ad libitum access to water and chow (5001, LabDiet) or a 60% high fat diet (HFD; D12492, Research Diets) and when applicable had ad libitum access to kaolin pellets (K50001, Research Diets). Rats were exposed to kaolin for at least 5 days prior to measuring kaolin consumption in pica testing. Except for studies conducted in the BioDaq, for all feeding studies rats were housed in hanging wire cages to allow for accurate measurement of food spillage.

Adult male shrews (Suncus murinus) weighing ˜50-80 g, were bred and maintained in the De Jonghe Lab (University of Pennsylvania). These animals were offspring from a colony previously maintained at the University of Pittsburgh Cancer Institute (Dr. Charles Horn); a Taiwanese strain derived from stock originally supplied by the Chinese University of Hong Kong). Shrews were single housed in plastic cages (37.3×23.4×14 cm, Innovive) under a 12 h-light: 12 h-dark cycle in a temperature- and humidity-controlled environment. Animal were fed ad libitum with a mixture of feline (75%, Laboratory Feline Diet 5003. Lab Diet) and mink food (25%, High Density Ferret Diet 5LI4, Lab Diet) and had ad libitum access to tap water.

Experiments were conducted under the National Institutes for Health Guide for the Care and Use of Laboratory Animals and all procedures were approved by the Institutional Animal Care and Use Committee at the University of Pennsylvania.

### Surgeries

For cannula implantation, rats were anesthetized by intraperitoneal injection of a mixture containing ketamine (90 mg/kg, Butler Animal Health Supply), xylazine (2.7 mg/kg, Anased), and acepromazine (0.64 mg/kg, Butler Animal Health Supply) (KAX) and then placed into a stereotaxic apparatus. Each rat was stereotaxically implanted with a guide cannula (26-ga, Plastics One) aimed at the fourth ventricle (guide cannula coordinates: on midline, 2.5 mm anterior to occipital suture, 5.2 mm ventral to skull; internal cannula aimed 7.2 mm ventral to skull) or the lateral ventricle (guide cannula coordinates: 1.5 mm lateral to midline, 0.9 mm posterior to bregma, 1.8 mm ventral to skull; internal cannula aimed 3.8 mm ventral to skull). For all cannulas, dummies (no projection beyond guide) were inserted in the guide cannula and left until infusions were performed. For all surgeries, rats received post-operative temperature support and analgesia was provided immediately following surgery and for two post-operative days (2 mg/kg meloxicam).

### Food and Kaolin Intake Studies

For all studies measuring food intake following drug treatment, central injections were given at volume of 2 μL were administered using a Hamilton syringe terminating in an injector tip extending 2.0 mm beyond the guide cannula, and intraperitoneal injections were given based on body weight (0.1 mL/100 μg body weight). For acute treatment days, rats were food deprived 2 hours before the dark cycle and injections were done immediately prior to the dark cycle onset. Food and kaolin intake was measured 1, 3, 6, and 24 hours after injections were completed and food crumbs were weighed and accounted for between each timepoint. Body weight was measured during injections and 24 hours after. Injection treatments were organized in a counterbalanced, within-subjects design and separated by >72 h. For chronic intake studies, once daily drug injections and recording of body weight, food, and kaolin intake were performed every 24 h immediately prior to the dark cycle onset. For meal pattern analysis, rats living in a Biodaq system (Research Diets, Inc) were injected similarly. The BioDaq system records on a second-by-second basis for undisturbed measurements of episodic food intake. Individual bouts are initiated by the animal at onset and termination of feeding; bouts are separated by 5 second inter-bout interval (IBI). A meal is defined as at least one bout with a minimum meal size of 0.02 μg and separated by a 5-minute undisturbed inter-meal interval (IMI). Cumulative food intake, the number of meals, time spent consuming meals, average meal size (g/meal), and average meal length (sec/meal) was calculated for hours 0-1, 0-6, 6-12, and 12-24 relative to drug injection. Additionally, cumulative food intake (g), number of bouts, time in bouts, number of meals, and time in meals were calculated for 20 min intervals for the first 3 hours post injection in chow fed rats and for intervals of one hour for 24 hours post injections in HFD fed rats.

### Drugs

Most drugs (ODN, TDN, OP, AntOP, SUODN04, SUODN05) used in these studies were synthesized by the Doyle lab at Syracuse University. Drugs that were purchased commercially include: Rat recombinant diazepam binding inhibitor (DBI) protein (LS-G136996, Lifespan Biosciences) and exendin-4 (11096, Caymen Chemical) which were dissolved in artificial cerebrospinal fluid (aCSF, Harvard Apparatus), liraglutide (24727, Caymen Chemical) which was dissolved in 40 mM Tris HCl buffer (pH8) 0.02% Tween-80, and rabbit anti-DBI primary antibody (ab231910, Abeam) used to neutralize endogenous DBI which was not diluted and the appropriate vehicle used was a 1:1 solution of glycerol and aCSF.

### Hindbrain DBI Immunohistochemistry and Quantification

Rats maintained on chow or HFD were either ad libitum fed or fasted for 24 h. All rats received a 4^{th} ventricle injection of aCSF or exendin-4 (0.3 μg/2 μL) 90 minutes prior to sacrifice. Rats were deeply anesthetized with KAX and transcardially perfused with 0.1 M phosphate-buffered saline (PBS; pH 7.4) followed by 4% paraformaldehyde in 0.1 M PBS on ice. Brains were removed from the crania and post-fixed in 4% paraformaldehyde for 24 h, then stored in 20% sucrose in 0.1 M PBS at 4° C. until sunk. Coronal dorsal vagal complex (DVC) sections (30 μm) were sliced and collected directly onto slides (12-550-15; Superfrost Plus, Fisher Scientific) using a cryostat and stored at −80° C. until the start of immunohistochemistry (IHC). Briefly, tissue was washed with 0.1 M PBS 3 times for 8 minutes and then incubated in blocking solution [5% normal donkey serum (Jackson Immunoresearch) in PBST; PBS with 0.3% triton X] followed by overnight incubation in rabbit anti-DBI primary antibody (1:500; ab231910, Abcam) and chicken anti-vimentin primary antibody (1:2000; ab24525, Abcam) at 4° C. The next morning slides were washed 3 times for 8 minutes in PBST and then incubated in donkey anti-rabbit fluorescent secondary antibody (1:500; AlexaFluor 647, Jackson Immunoresearch) and donkey anti-chicken fluorescent secondary antibody (1:500; AlexaFluor 488, Jackson Immunoresearch) for 3 h at room temperature. Finally, slides were washed 3 times for 5 minutes in PBST and one time in PBS before coverslipped with antifade mounting media with DAPI (H-1200, Vector Laboratories, Inc.). Slides were visualized using fluorescence microscopy (BZ-X810, Keyence). Images analysis to quantify the fluorescent intensity of DBI protein staining in the nucleus solitary tractus (NTS) and area postrema (AP) as well as the % colocalization of DBI and vimentin staining in the AP, subpostrema, and 4^{th} ventricle boarder was done using the HALO@FISH-IF and Area Quantification FL modules.

Quantitative Real-Time (qPCR) Studies

Chow-maintained rats received a 4^{th} ventricle injection of aCSF or exendin-4 (0.3 μg/2 μL) 90 minutes prior to sacrifice. Brains were rapidly removed, flash-frozen in −70° C. isopentane, and stored at −80° C. until processing. Micropunched tissue from the DVC was collected from each brain. Total RNA was extracted from tissue from each site using TRIzol (Invitrogen) and the RNeasy kit (Qiagen). The Advantage RT-for-PCR Kit (Clontech) was used to synthesize cDNA from 200 ng of total RNA. Relative mRNA levels of DBI were quantified using quantitative real-time PCR. Rat GapDH (VIC-MGB) was used as an internal control. PCR reactions were completed using TaqMan gene expression kits (DBI: Rn00821402_g1 and GapDH: Rn01775763_g1) and PCR reagents from Applied Biosystems. Samples were analyzed with the QuantStudio 6 Pro system (Applied Biosciences). Relative mRNA expression calculations were completed using the comparative threshold cycle method.

### Hindbrain Glucose Sensing Studies

Chow-maintained rats were food deprived 2 hours before the dark cycle and 4′ ventricle injections were done immediately prior to the dark cycle onset. Baseline glucose values were taken from tail vein blood by glucometer (Concur) prior to injections and 30 and 60 minutes after injections. In one study rats received a pretreatment injection of vehicle (aCSF) or OP (20 μg/2 VL) followed by treatment with vehicle or 5-thio-d-glucose (5-TG; 210 μg/2 μL). In the other study rats received a pretreatment injection of vehicle or AntOP (20 μg/2 μL) followed by treatment with vehicle or D-glucose (5.5M in 3 VL). Food was returned 1 h after injections following measurement of the last blood glucose concentrations and food intake was recorded 2, 4, 6, and 24 hours post injections. Body weight was measured during injections and 24 hours after.

### Peptide Synthesis and Purification

Solid-Phase Peptide Synthesis was performed on ProTide Rink amide resin (CEM Corporation cat #R002) using a microwave-assisted CEM Liberty Blue peptide synthesizer (Matthews, NC). Fmoc-protected amino acids were coupled to the resin using 0.25 M Oxyma Pure (CEM Corporation cat #S001) and 0.125 M N, N′-diisopropylcarbodiimide (Sigma-Aldrich cat #D125407) as the activator and activator base, respectively. Fmoc was removed between couplings with 20% Piperidine (Sigma-Aldrich cat #8.22299.0500). Global deprotection and cleavage of the peptides from the solid-support resin achieved using a CEM Razor instrument over a 40-minute incubation period at 40° C. in a mixture of 95% TFA (Sigma-Aldrich cat #8.08260.2501), 2.5% TIPS (Sigma-Aldrich cat #233781), and 2.5% water. Peptides were purified on an Agilent 1200 series High-Performance Liquid Chromatography (HPLC) instrument (10-75% HPLC-grade acetonitrile (VWR cat #BDH83639.400) for 20 minutes at 2 mL/min flow rate using an Agilent Zorbax C18 column (5 μm, 9.4×250 mm) tracked at 220 nm.

### Central ODN Antagonizes Relaxin-3 Methods

Rats were stereotaxically implanted with a bilateral guide cannula (26-ga, Plastics One) aimed at the nucleus incertus (guide cannula coordinates: ±0.5 lateral to midline, 9.5 mm anterior to bregma, 5.8 mm ventral to skull; internal cannula aimed 7.8 mm ventral to skull). Recombinant human relaxin-3 was purchased (130-10, PreproTech Inc.) and ODN was synthesized by the Doyle laboratory. Injection treatments [pretreatment with ODN (10 μg) or vehicle and treatment with relaxin-3 (32.4 nmol) or vehicle] were organized in a counterbalanced, within-subjects design and separated by ≥72 h. Rats were food deprived 2 hours before the dark cycle and injections were done immediately prior to the dark cycle onset. All injection were given at a volume of 100 nL in aCSF at a rate of 20 nL/second using a micropump-depressed (PHD 2000, Harvard Apparatus) loaded with a Hamilton syringe terminating in an injector tip extending 2.0 mm beyond the bilateral guide cannula implanted at the nucleus incertus coordinates above. 24 h food intake was recorded in a Biodaq system (Research Diets, Inc). Body weight was measured during injections and 24 hours after.

DVC Tissue Extraction from Male Sprague Dawley Rats

Male Sprague Dawley rats were anesthetized by isoflurane and rapidly de-capitated, the brains removed and flash frozen in −70° C. isopentane and stored at −80° C. Using a cryostat, the DVC (comprised of the area postrema, nucleus tractus solitarius, and dorsal motor nucleus of the vagus) of the rat brainstem was micropunched 1 mm3 per subnuclei at the level of the AP and pooled together in a cryovial and restored at −80° C. for Protein/GPCR tissue extraction procedures below.

ODN-Bound Protein/GPCR Extraction Procedures from Rat DVC Tissue

Membrane proteins were extracted from rat brain tissue based on the protocol for tissues on the GPCR Extraction and Stabilization Reagent (GESR) (ThermoFisher Scientific. Rockford, Il). Briefly, the tissue samples were suspended in 1 mL of cold (4° C.) PBS and washed repeatedly. The PBS was decanted, and 1 mL of cold (4° C.) GESR was added to the tissue samples. The tissue samples were homogenized until an even suspension was obtained by pipetting up and down 15-20 times. The homogenate was transferred to a new tube and was incubated at 4° C. for 30 minutes with end-over-end mixing. The sample was centrifuged at 16,000×g for 20 minutes at 4° C. The supernatant containing stabilized protein receptors was saved and stored at 4° C. until being analyzed.

### Binding Assay

Binding analysis was done on a Nicoya Open Surface plasmon resonance instrument using a Nicoya streptavidin sensor chip. The coupling procedure was according to the streptavidin sensor chip protocol, including the steps of surface conditioning and surface activation. For ligand immobilization, ODN-biotin (20 μg/mL in the PBST pH 7 running buffer) was injected over channel 2 for a 5-minute interaction time. This process was repeated several times to ensure optimal immobilization. The supernatant from the GPCR extraction procedure was injected over channels 1 and 2 of the chip, and a background-corrected binding curve was obtained. The chip was then soaked for 16 hours in 5 mL of MeOH at 4° C. An electron absorption spectrum of the MeOH used to soak the chip was obtained using a Nanodrop One. The remaining MeOH was mixed with water, freeze-dried, and sent out for MS/MS sequencing.

The following examples are provided to illustrate certain embodiments of the invention. They are not intended to limit the invention in any way.

### Example 1

### Synthesis and Testing of ODN Peptides and Derivatives

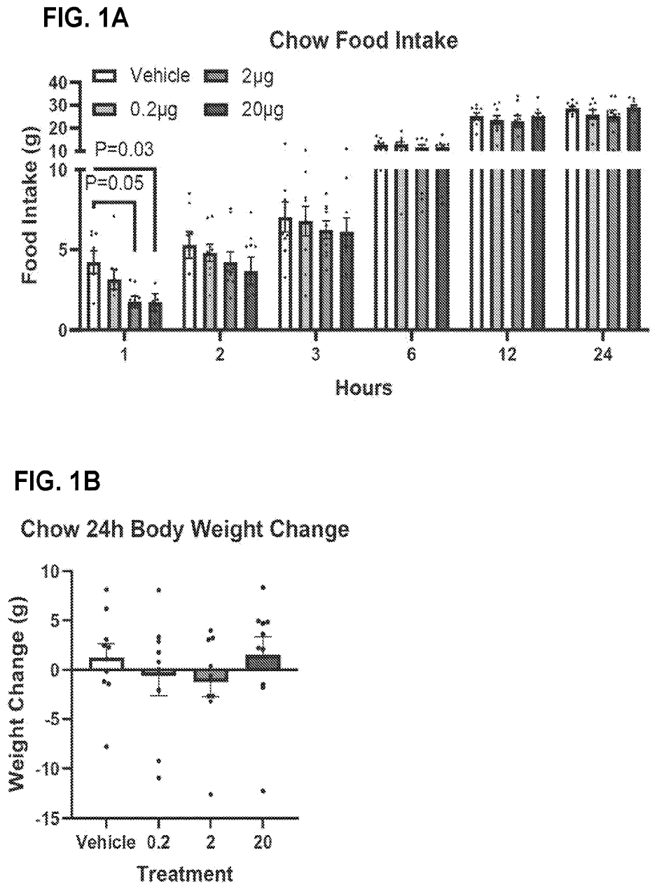

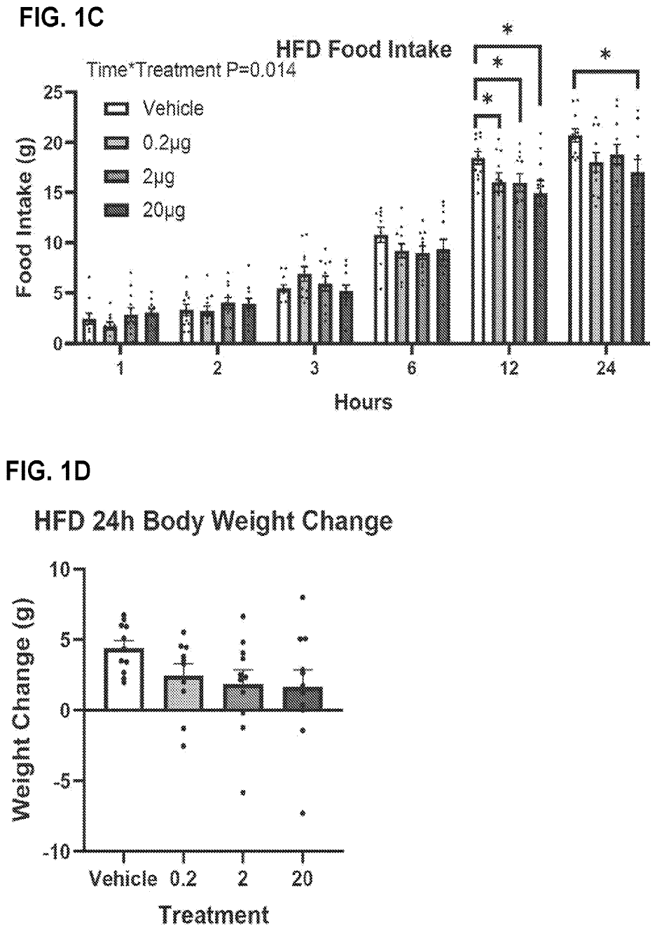

Central administration of ODN at 0.2, 2, and 20 μg/2 μL in the 4′ ventricle dose dependently decreases food intake in the first hour after injections in chow-maintained rats (FIG. 1A) without affecting 24 h body weight change (FIG. 1B), and also dose dependently decreases food intake in diet-induced obese (DIO) 60% high-fat diet (HFD)-maintained rats at hours 12 and 24 post injections (FIG. 1C) while non-significantly decreasing 24 h body weight change (FIG. 1D). These data support that intracerebroventricular administration of ODN dose-dependently suppresses intake of both chow and HFD, although with differences in time course of the anorectic response. ODN acts acutely in lean chow-maintained rats but is longer-lasting and more effective at suppressing HFD intake in obese rats.

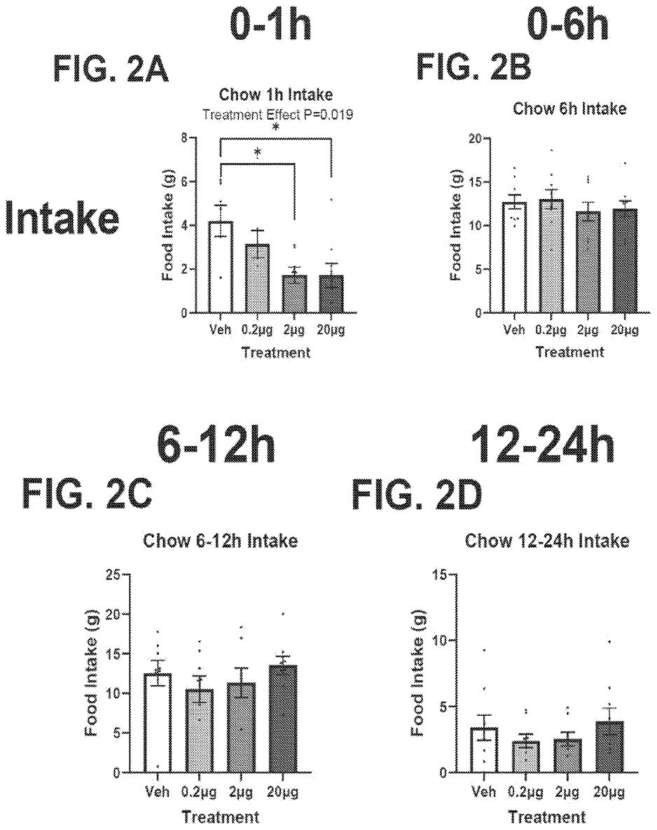

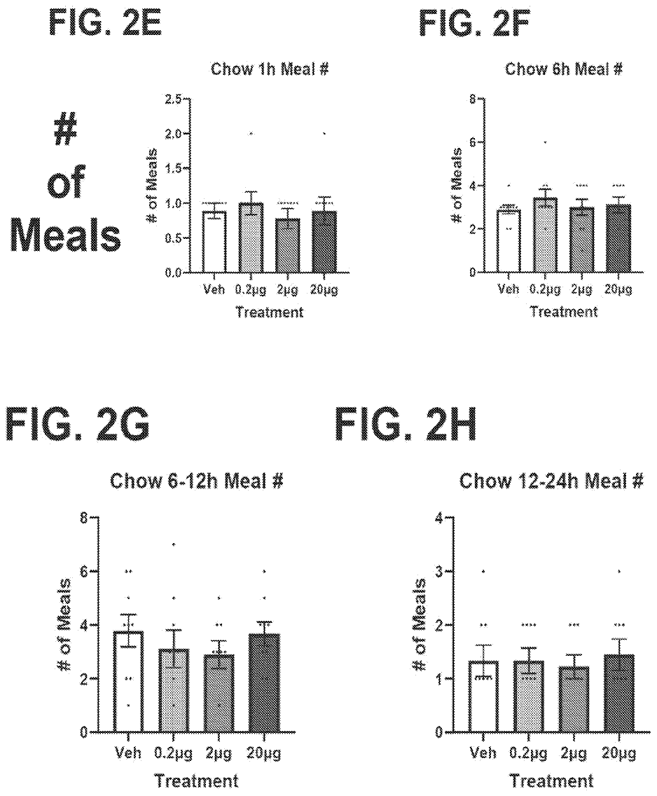

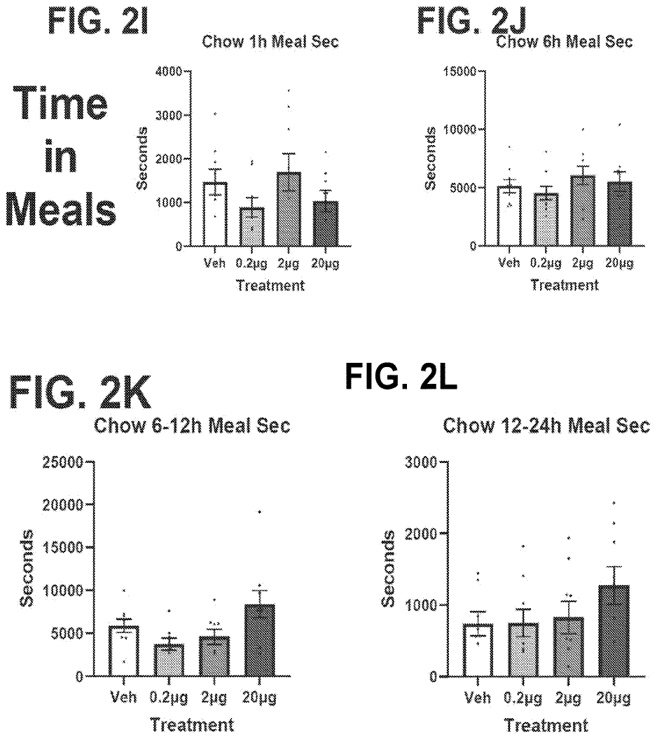

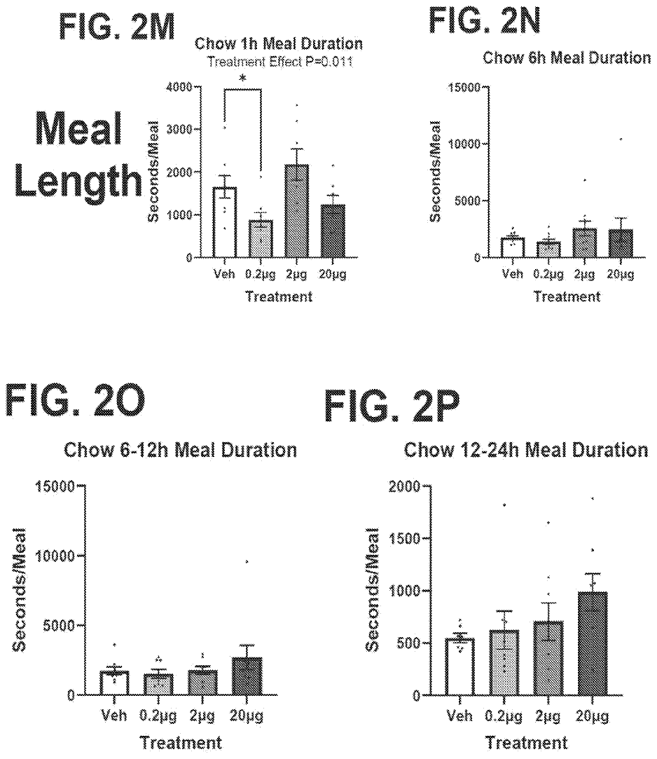

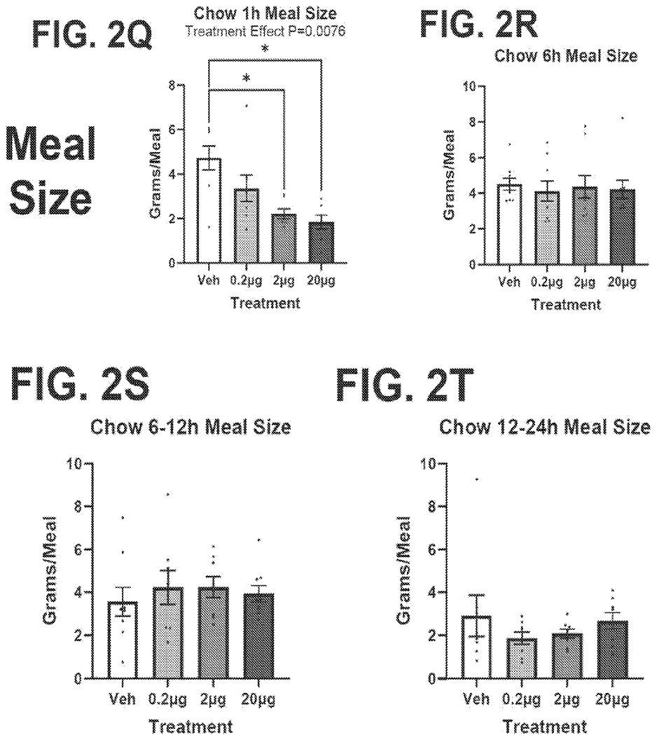

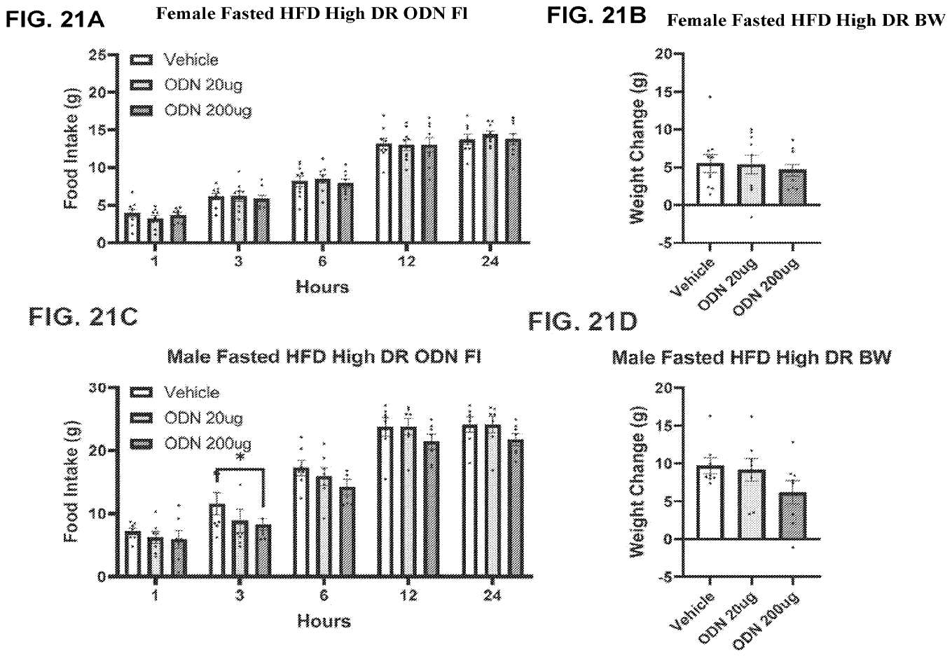

Meal pattern data examined the amount of food intake (grams), the number of meals, time spent eating meals (seconds), meal length (seconds/meal), and meal size (grams/meal) in chow-maintained rats at time intervals 0-1 hours, 0-6 hours, 6-12 hours, and 12-24 hours post ODN injection. ODN dose dependently decreased the amount of food eaten in the first hour (FIG. 2A) but not any other time frame (FIGS. 2B-2D). ODN did not impact the number of meals eaten at any time frame (FIGS. 2E-2H), or the time spent in meals (FIGS. 21-2L). The meal length was decreased by 0.2 μg ODN in hour 1 but not in any other time frame (FIGS. 2M-2P). ODN dose dependently decreased meal size in the first hour (FIG. 2Q) but not any other time frame (FIGS. 2R-2T). These data support that in chow-maintained rats, ODN suppresses food intake in the first hour post injections by decreasing meal size.

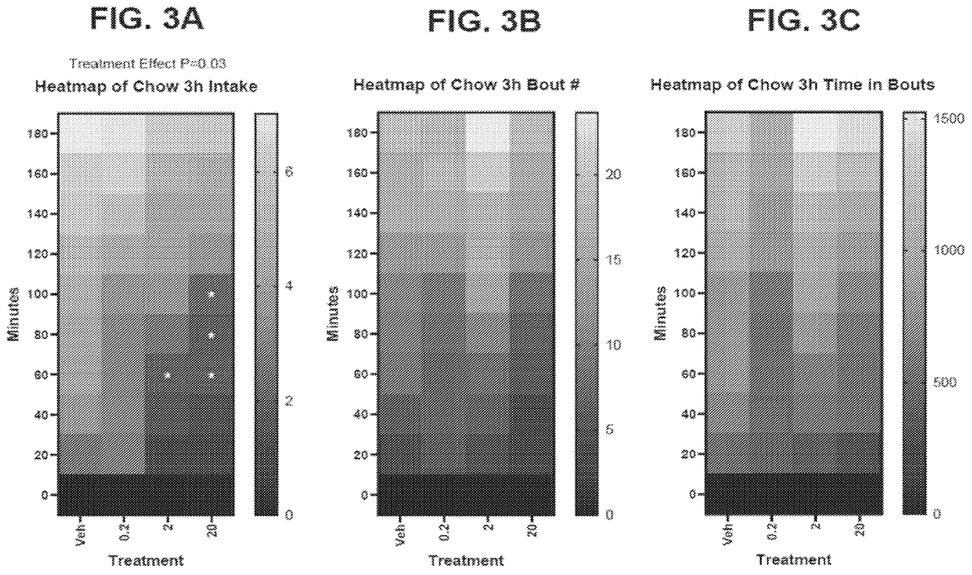

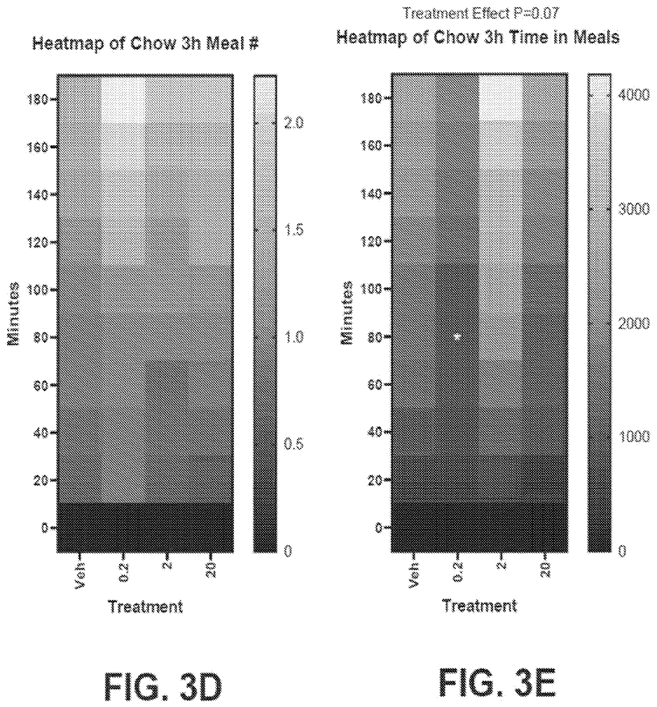

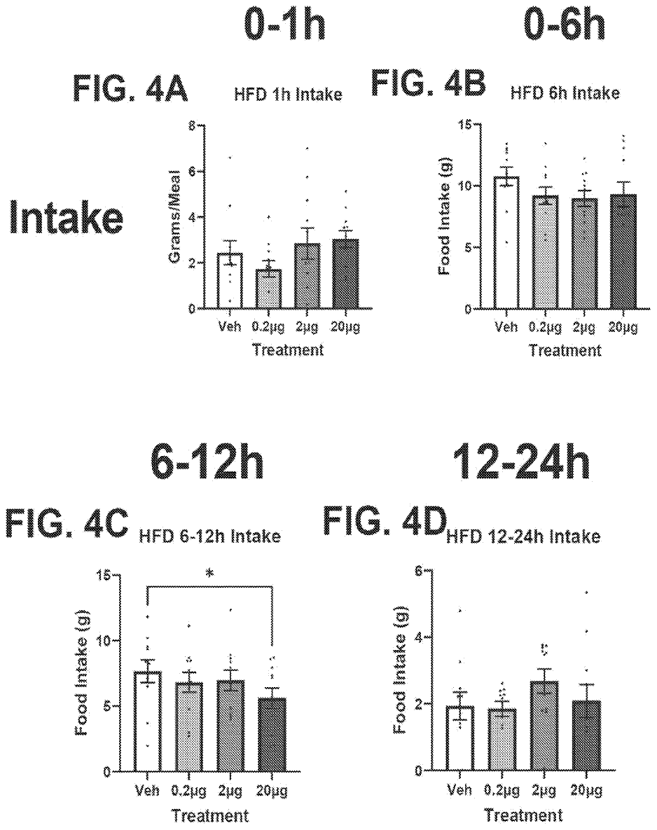

Heat map representation of food intake (FIG. 3A, grams). bout number (FIG. 3B), time spent in bours (FIG. 3C, seconds), meal number (FIG. 3D), and time spent in meals (FIG. 3E, seconds) during the first 3 hours post injections in chow-maintained rats. Compared to vehicle, 2 and 20 μg ODN decreased food intake 60 minutes after injections and 20 μg ODN also decreased food intake at 80 and 100 minutes (FIG. 3A). The number of bouts, time in bouts, and number of meals was not affected by ODN (FIGS. 3B-3D), and 0.2 μg ODN decreased the time spent in meals at 80 minutes post injection relative to vehicle (FIG. 3E).

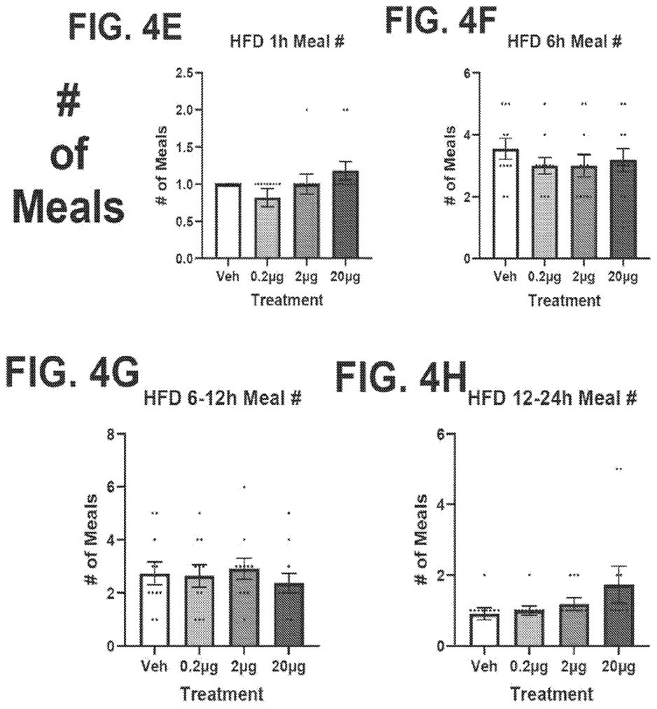

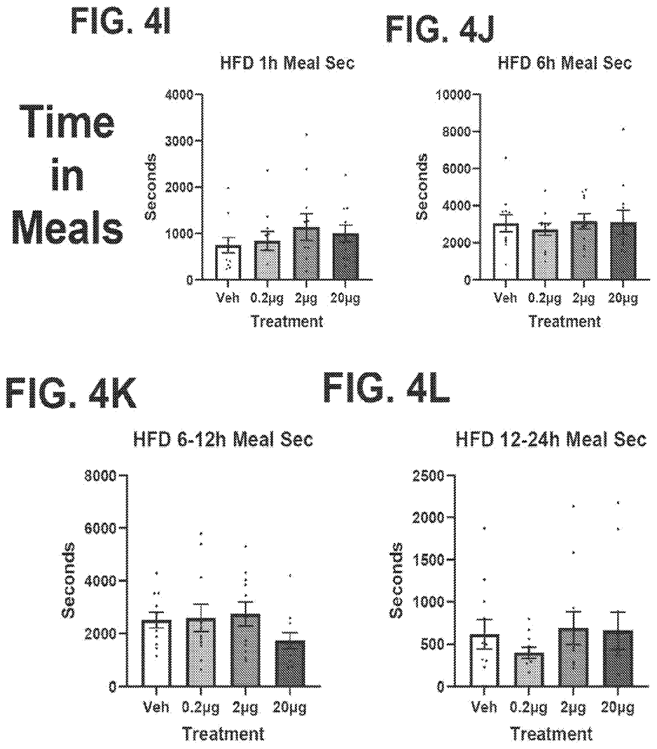

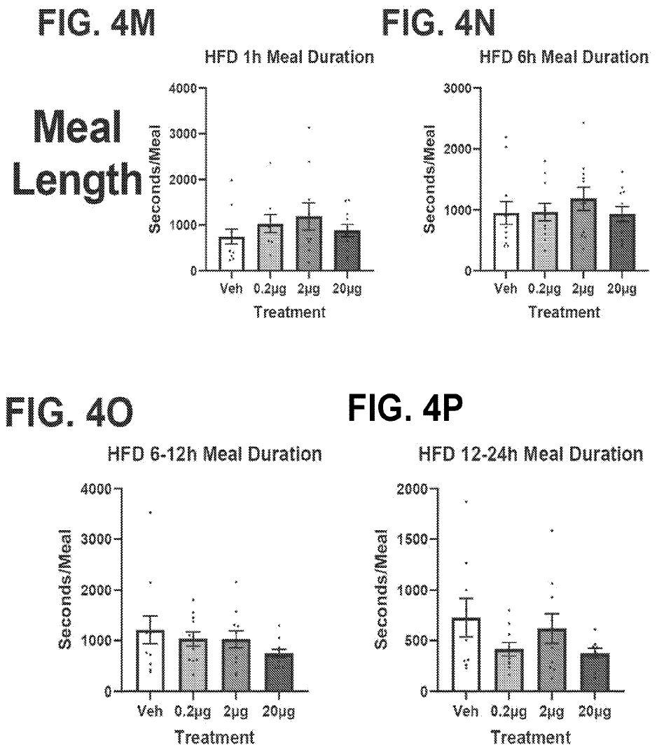

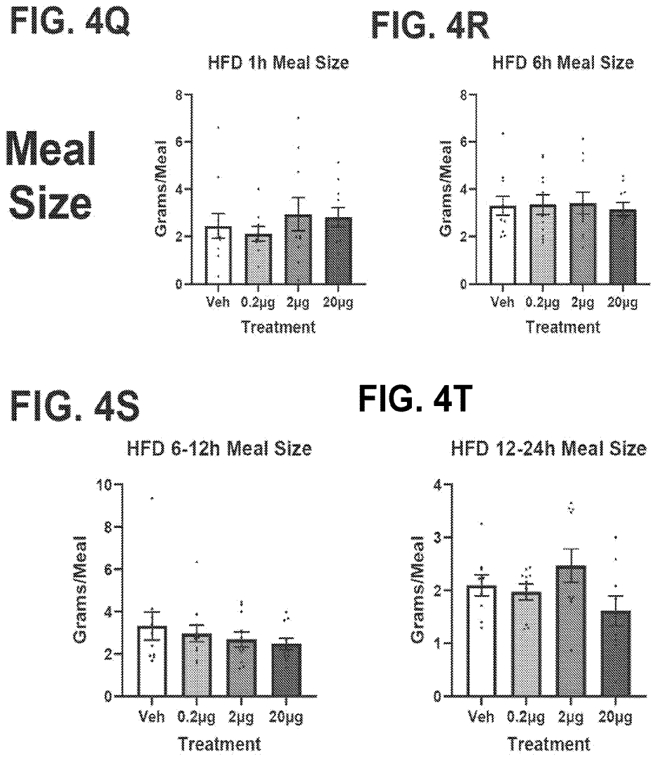

Meal pattern data examined the amount of food intake (grams), the number of meals, time spent eating meals (seconds), meal length (seconds/meal), and meal size (grams/meal) in HFD-maintained rats at time intervals 0-1 hours, 0-6 hours, 6-12 hours, and 12-24 hours post ODN injection. 20 μg ODN decreased the amount of food eaten during 6-12 hour post injections but no other time frame (FIG. 4A-4D). ODN did not affect meal pattern data at any time frame (FIG. 4E-4T).

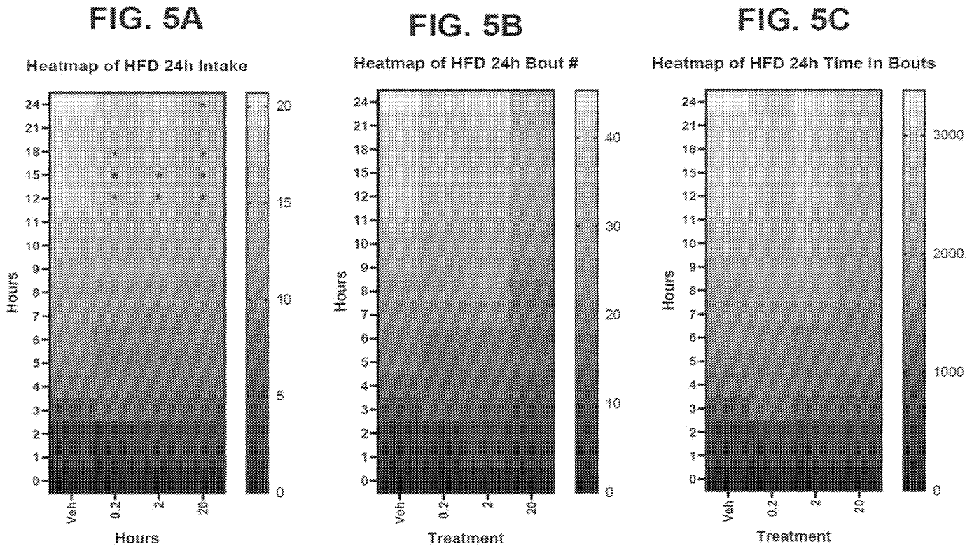

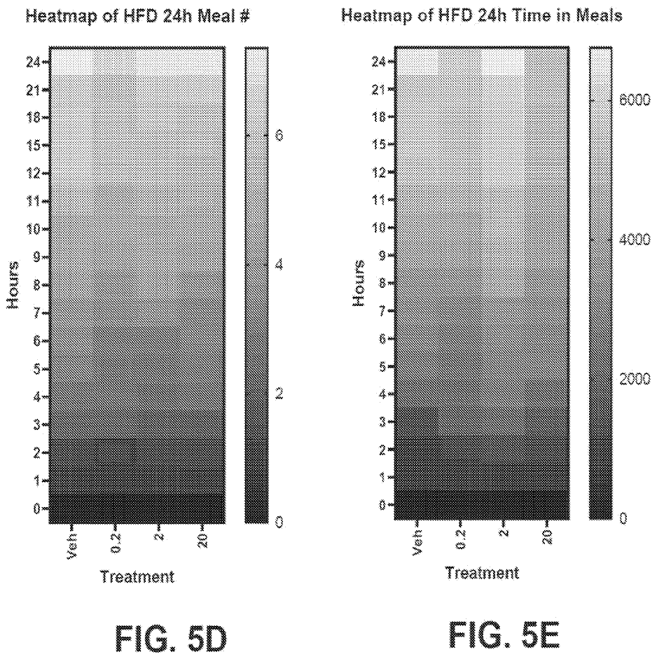

Heat map representation of food intake (FIG. 5A, grams). bout number (FIG. 5B), time spent in bours (FIG. 5C, seconds), meal number (FIG. 5D), and time spent in meals (FIG. 5E, seconds) during the 24 hours post injections in HFD-maintained rats. 0.2, 2, and 20 μg ODN decreased food intake at 12 and 15 hours post injection, 0.2 and 20 μg also at 18 hours, and 20 μg ODN also at 24 hours (FIG. 5A). ODN did not significantly alter the number of bouts, time in bouts, number of meals, and time in meals (FIGS. 5B-5E).

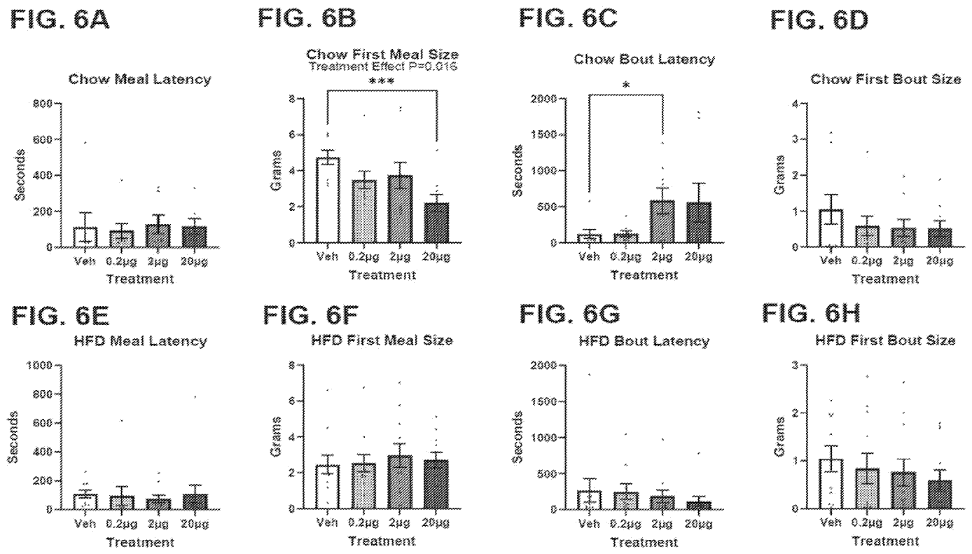

Meal pattern data on the first bout and meal consumed post ODN injection in chow and HFD-maintained rats is shown in FIG. 6A-6H. Consistent with acute effect of ODN in chow-maintained rats, ODN did not alter the latency to the first meal (FIG. 6A) but 20 μg ODN decreased the size of the first meal (FIG. 6B). 2 μg ODN increased the latency to the first bout consumed (FIG. 6C) but did not alter the size of the first bout (FIG. 6D). Consistent with the delayed impact of ODN in obese rats, ODN did not alter the latency to consume of the size of the first meal or bout (FIGS. 6E-6H).

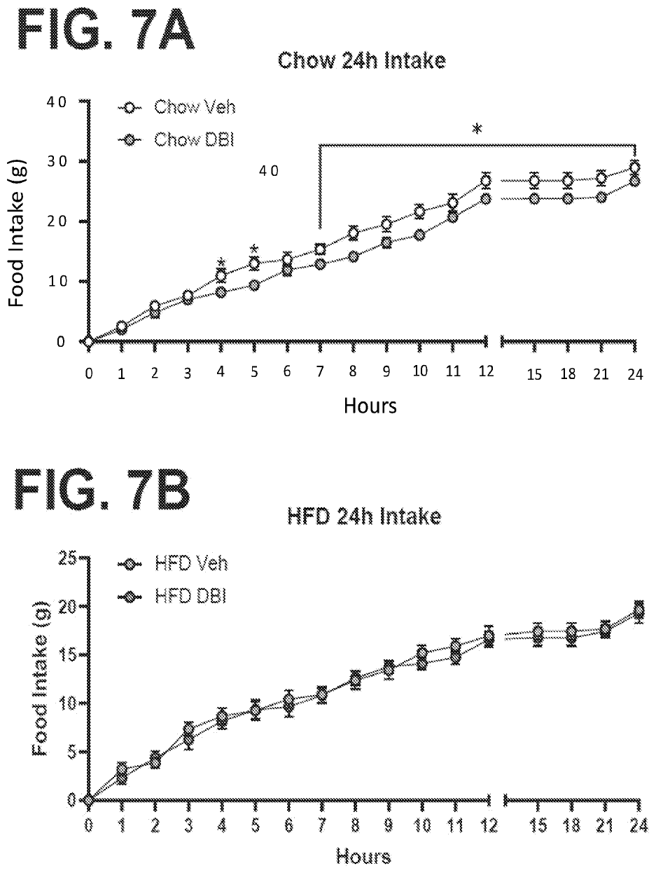

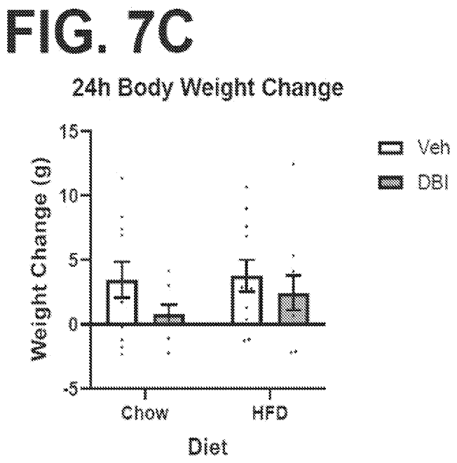

ODN is a cleavage product of DBI and to understand how the parent peptide DBI regulates food intake in chow and HFD-maintained rats we injected recombinant DBI protein into the 4′ ventricle. In chow-maintained rats, DBI significantly reduced food intake at 4, 5, and 7-24 hours post injection (FIG. 7A). Surprisingly, DBI did not decrease food intake in HFD-maintained rats (FIG. 7B). 24 h body weight change was not significantly decreased in either diet group (FIG. 7C). These results show that while ODN is more effective in obese rats, exogenous DBI does not suppress food intake, suggesting there may be some dysfunction in cleaving DBI to ODN in obesity that can be targeted to improve endogenous ODN signaling.

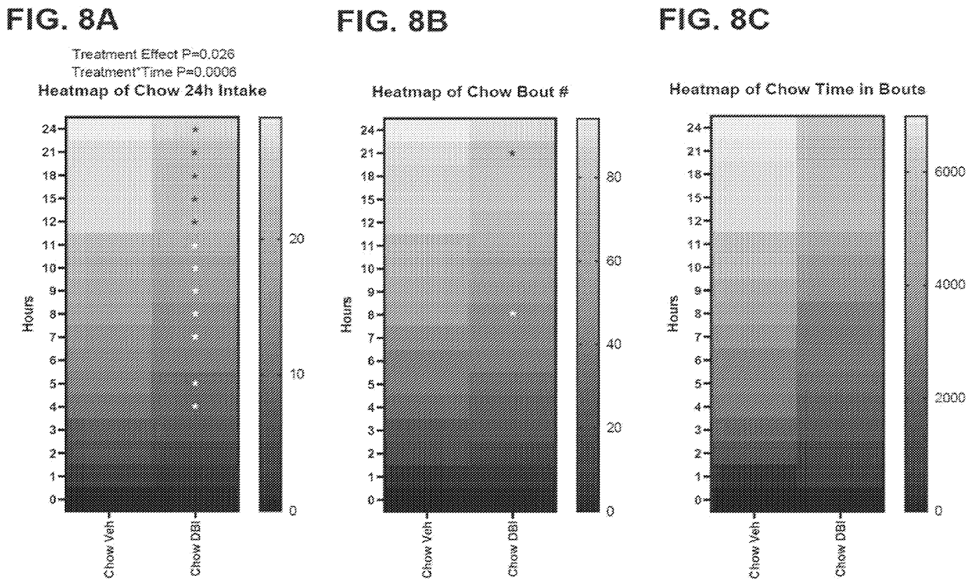

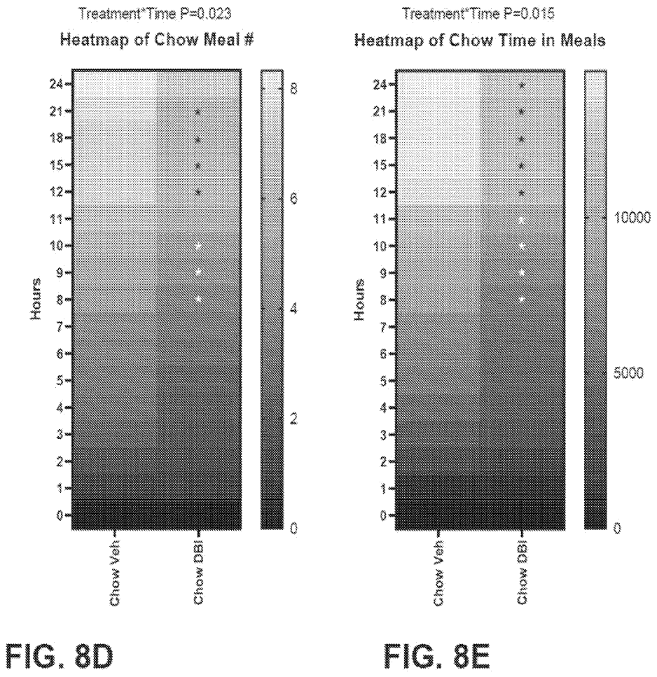

Heat map representation of food intake (FIG. 8A, grams), bout number (FIG. 8B), time spent in bours (FIG. 8C, seconds), meal number (FIG. 8D), and time spent in meals (FIG. 8E, seconds) during the 24 hours post recombinant DBI injections in chow-maintained rats is shown. DBI decreased food intake at 4, 5, and 7-24 hours post injection (FIG. 8A), decreased the number of bouts at 8 and 21 hours post injection (FIG. 8B), did not alter the time spent in bouts (FIG. 8C), decreased the number of meals at 8-10 and 12-21 hours post injection (FIG. 8D), and decreased the time spent in meals 8-24 hours post injection (FIG. 8E). These data support that DBI protein strongly suppresses food intake by limiting the time spent eating meals and the number of meals consumed in chow-maintained rats.

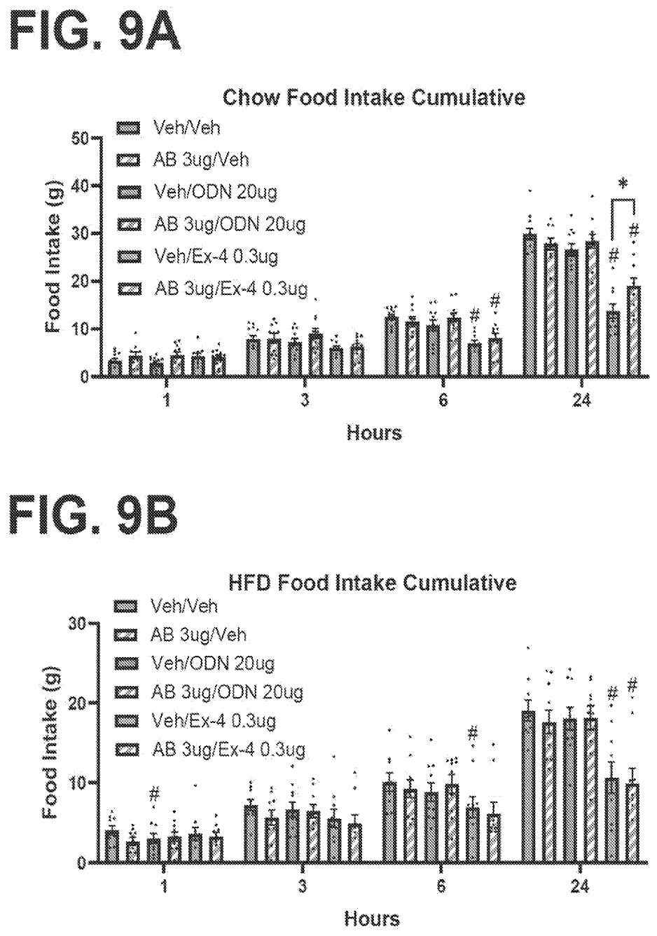

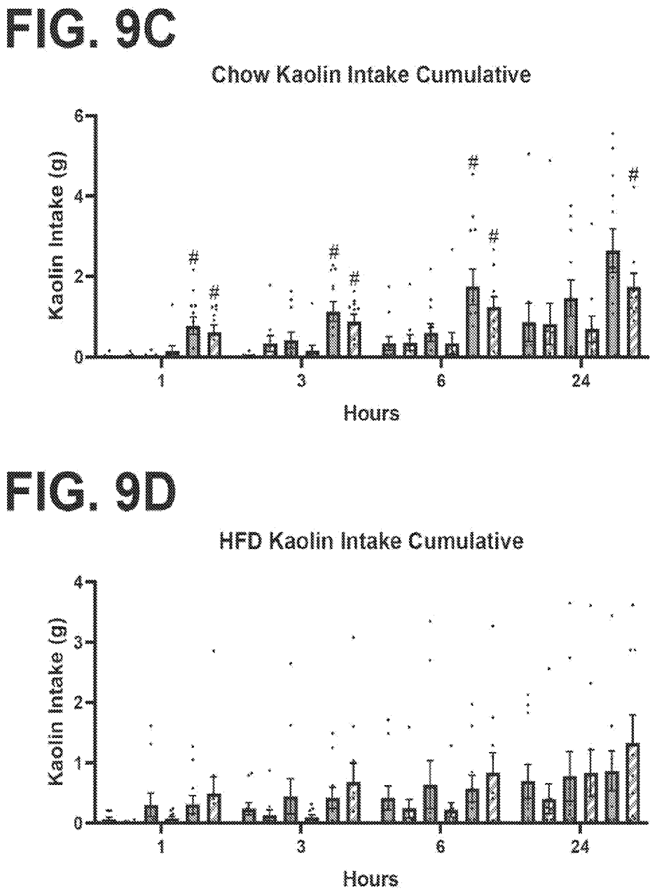

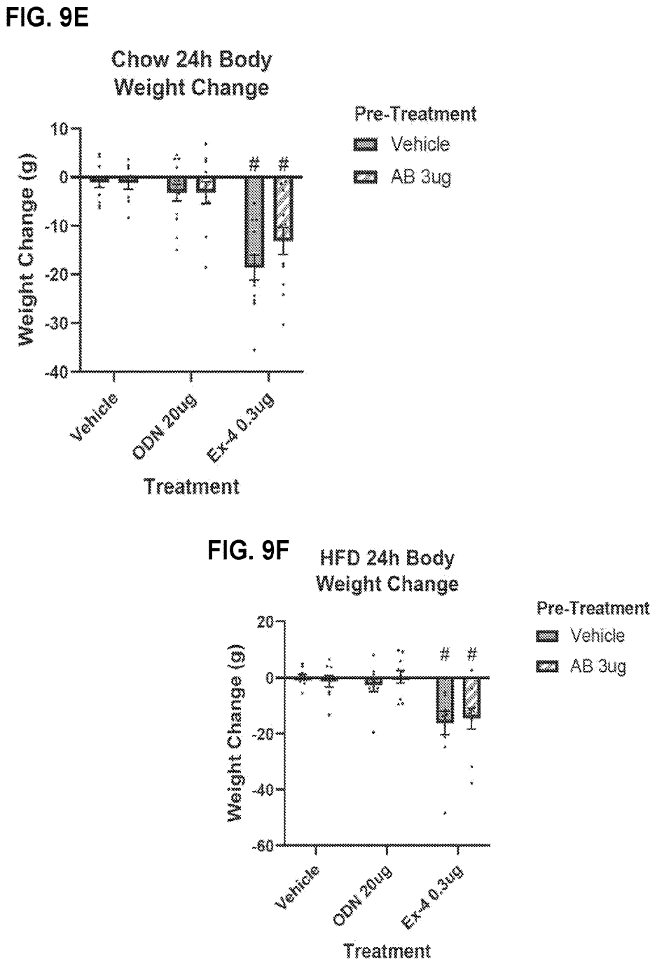

ODN expression and release has been shown to be regulated by nutritional state, being decreased by fasting and increased by refeeding and glucose. We hypothesized that glucagon-like peptide-1 (GLP-1), which is induced post-prandially, may also regulate ODN signaling. To test the contribution of ODN signaling to the anorectic actions of GLP-1, we pretreated chow and HFD-maintained rats in the 4′ ventricle with an antibody targeting DBI (AB) to neutralize endogenous DBI protein or vehicle before treatment with ODN, the GLP-1 receptor agonist, exendin-4 (Ex-4), or vehicle in the 4′ ventricle. AB pretreatment reduced the anorectic effect of Ex-4 at 24 hours post injections (FIG. 9A) but did not affect HFD intake (FIG. 9B). Kaolin intake was measured to assess pica behavior. As expected, Ex-4 increased kaolin consumption in chow-maintained rats relative to controls and this was numerically but not significantly reduced by AB pretreatment (FIG. 9C). There was no difference in kaolin intake between any treatment groups in HFD-maintained rats (FIG. 9D). Ex-4 decreased 24 h body weight change relative to controls in both chow and HFD-maintained rats independent of pretreatment (FIGS. 9E-9F).

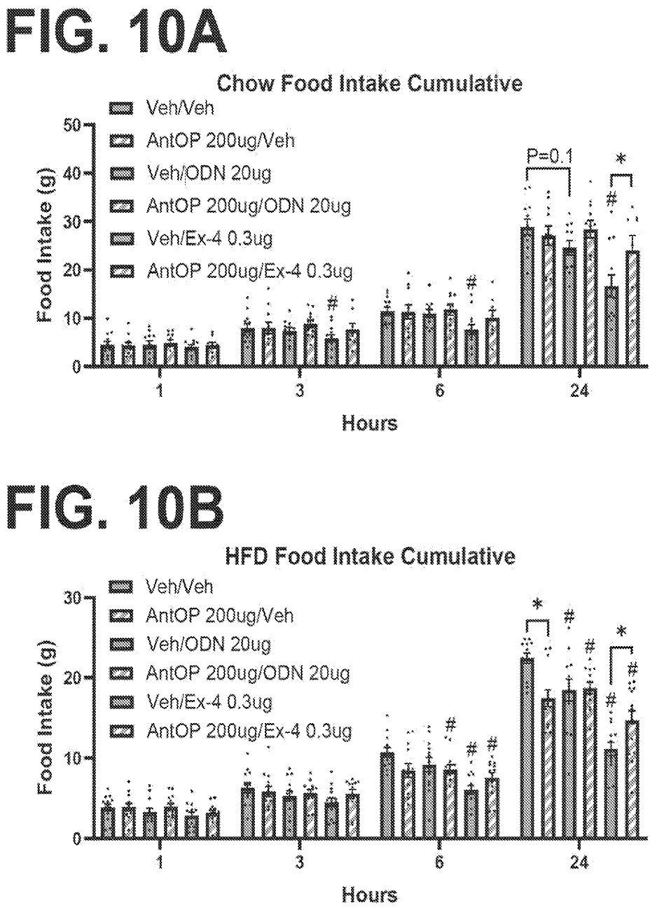

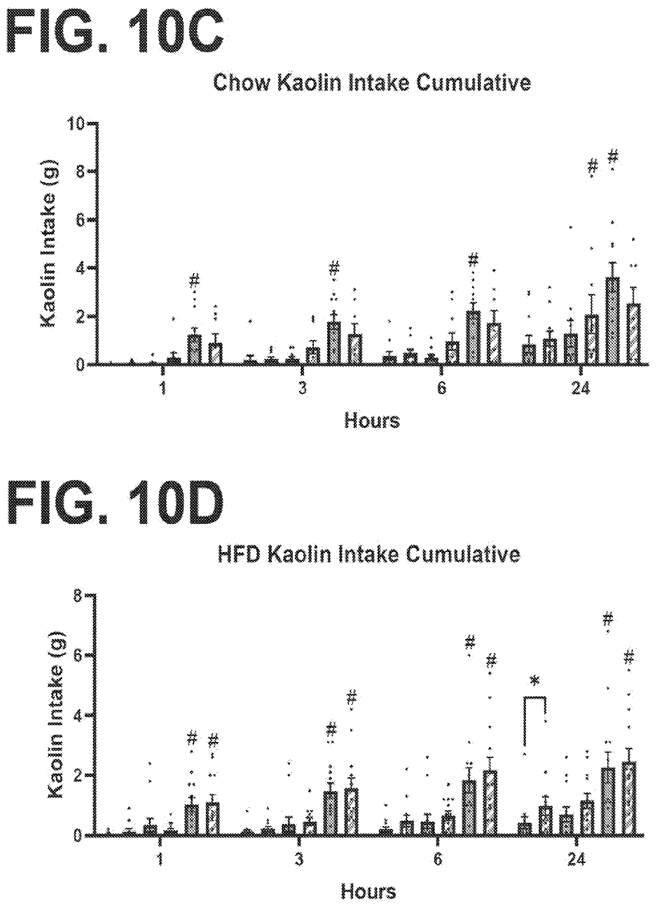

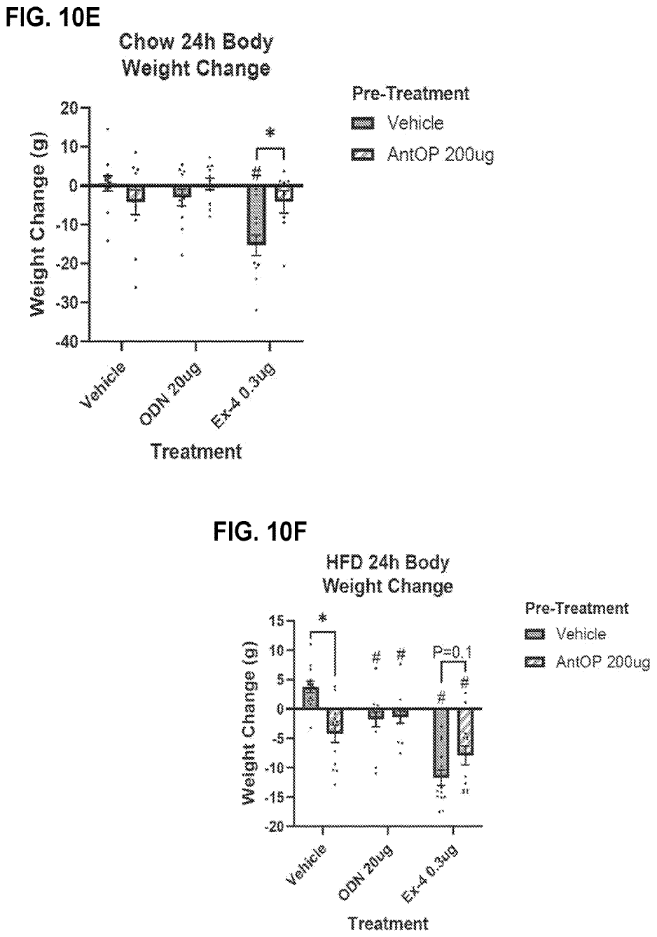

We repeated this study using a 411 ventricle administered ODN antagonist (AntOP) instead of the DBI targeted antibody and again demonstrated that AntOP pretreatment reduced the anorectic effect of Ex-4 in chow and HFD-maintained rats at 24 h post injections (FIGS. 10A-10B). Additionally, we observed that AntOP pretreatment alone decreased 24 h HFD intake (FIG. 10B). Ex-4 increased kaolin intake in chow and HFD-maintained rats relative to controls, and in chow rats AntOP pretreatment to Ex-4 did not significantly elevate kaolin intake relative to controls (FIGS. 10C-10D). In chow rats, AntOP pretreatment mitigated the Ex-4-induced robust decrease in body weight (FIG. 10E) and tended to do so in HFD-maintained rats (FIG. 10F). AntOP pretreatment alone also decreased 24 h body weight change in HFD rats (FIG. 10F).

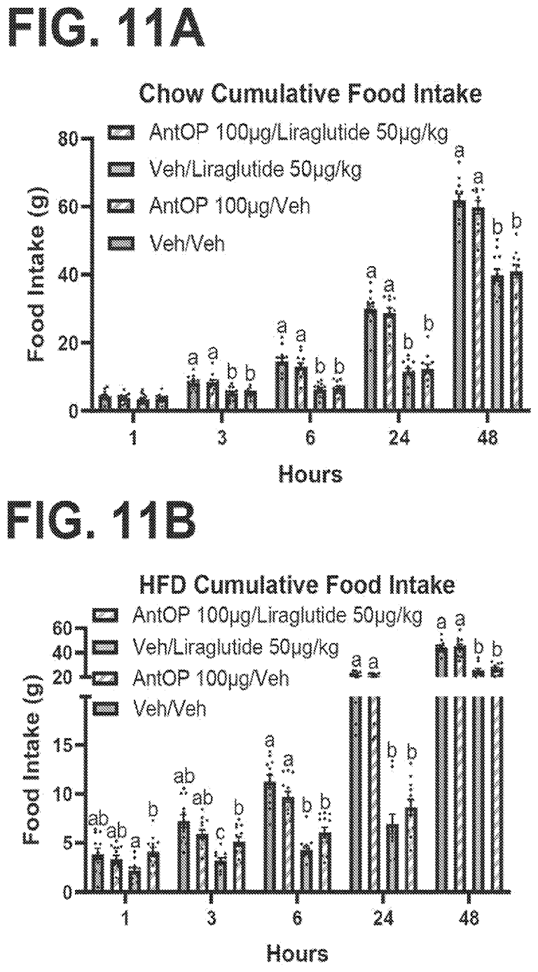

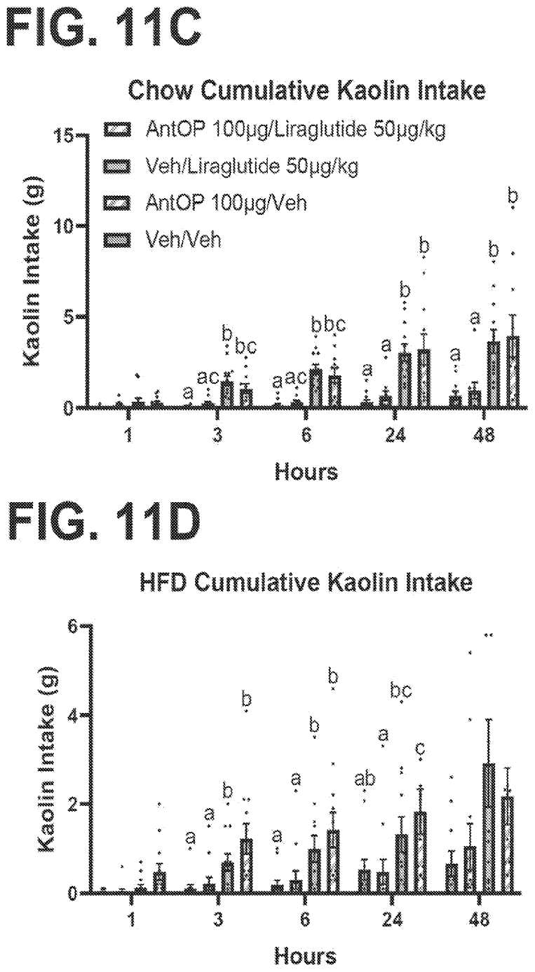

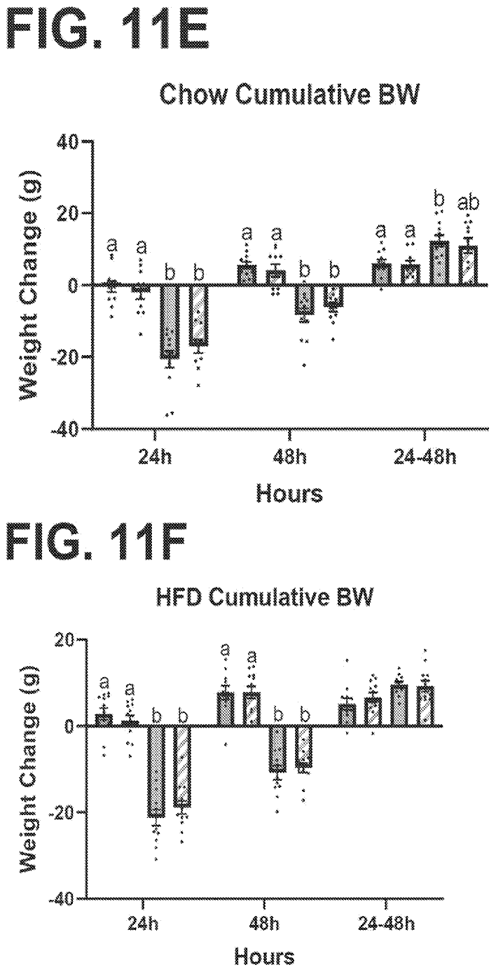

We next wanted to assess whether antagonizing ODN signaling could reduce the anorectic response to peripheral GLP-1 receptor agonists, so we administered AntOP or vehicle through the lateral ventricle and gave intraperitoneal injections of liraglutide or vehicle. Liraglutide decreased food intake in chow-maintained rats independent of pretreatment (FIG. 11A), and liraglutide decreased HFD intake which was attenuated by AntOP pretreatment at 1 and 3 hours post injections (FIG. 11B). Kaolin intake was increased, and body weight was decreased by liraglutide and not altered by AntOP pretreatment in chow and HFD-maintained rats (FIGS. 11C-11F). The results in FIGS. 9-11 demonstrate that the anorectic response to central and peripheral GLP-1 receptor agonists is partially mediated by central ODN signaling.

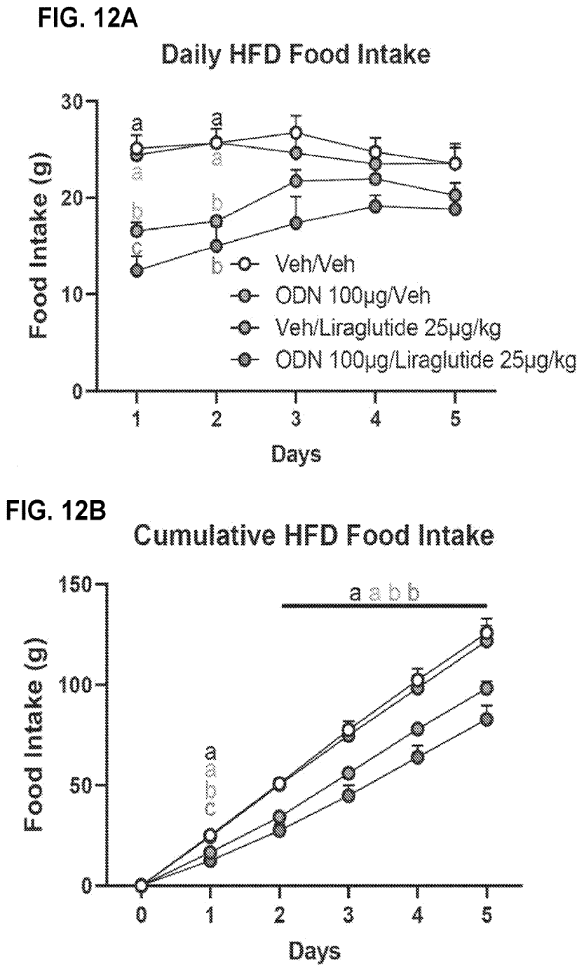

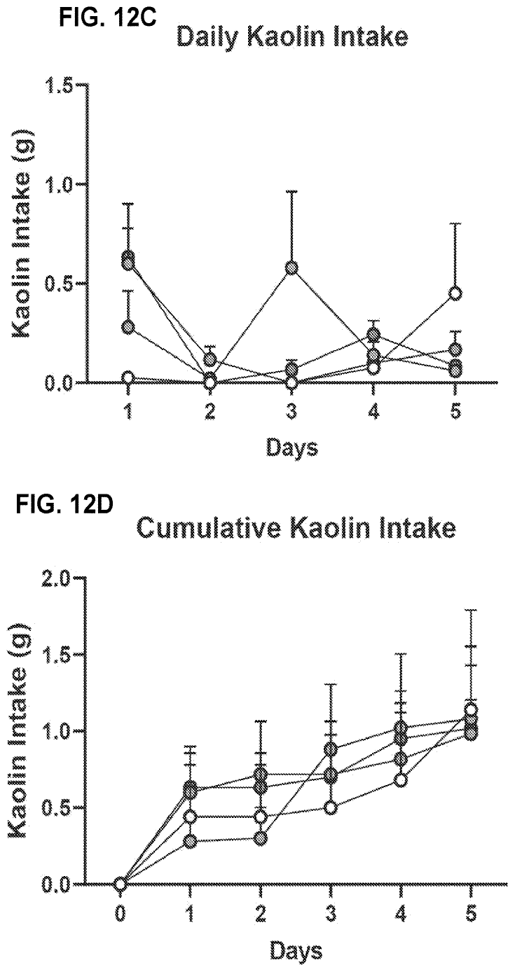

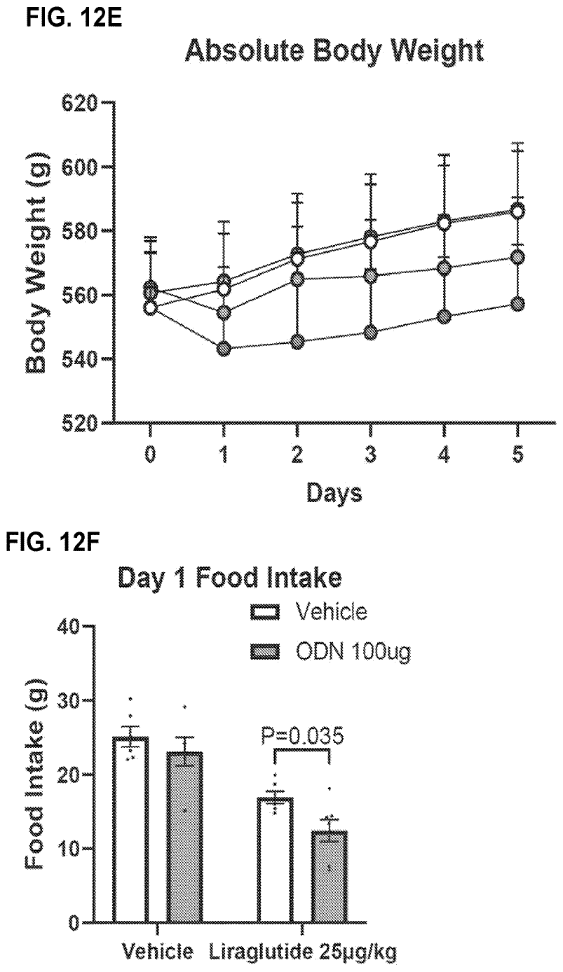

We next investigated whether ODN and GLP-1 receptor agonism can be cooperative to decrease food intake by injecting 4^{th} ventricle ODN or vehicle and intraperitoneal liraglutide or vehicle daily for 5 days. Liraglutide decreased daily food intake on day 1 and 2 relative vehicle and cumulative food intake on days 1-5, and ODN enhanced food intake suppression with liraglutide on day 1 (FIGS. 12A-12B). Daily and cumulative kaolin intake and daily body weight was not different between any treatments (FIGS. 12C-12E). Day one food intake was isolated and shows that while ODN alone did not alter food intake, it greatly enhanced the anorectic effect of liraglutide (FIG. 12F). These data suggest that as we propose ODN action may be downstream of GLP-1 signaling, and thus, GLP-1 receptor agonists do not maximally stimulate ODN signaling. This finding indicates that ODN agonists can be combined with GLP-1 agonists to provide more effective appetite control.

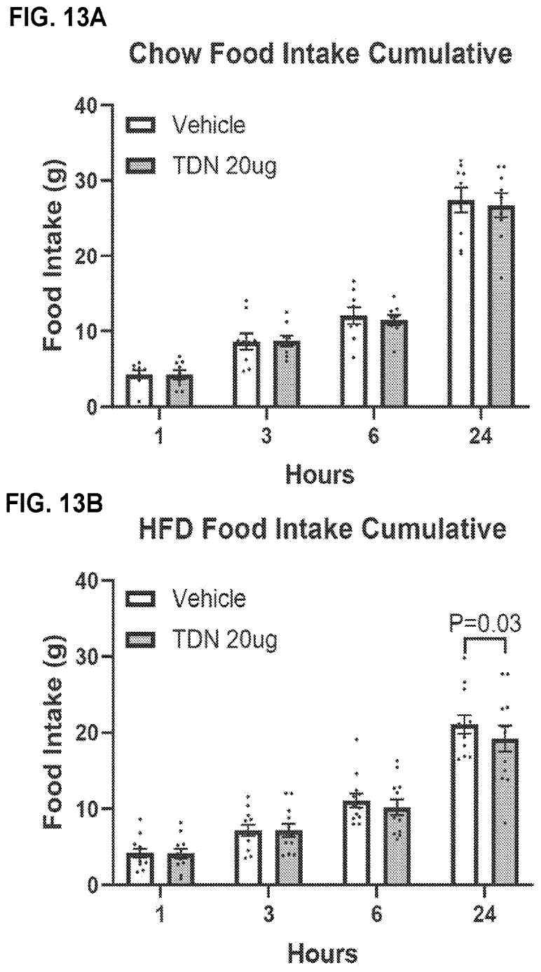

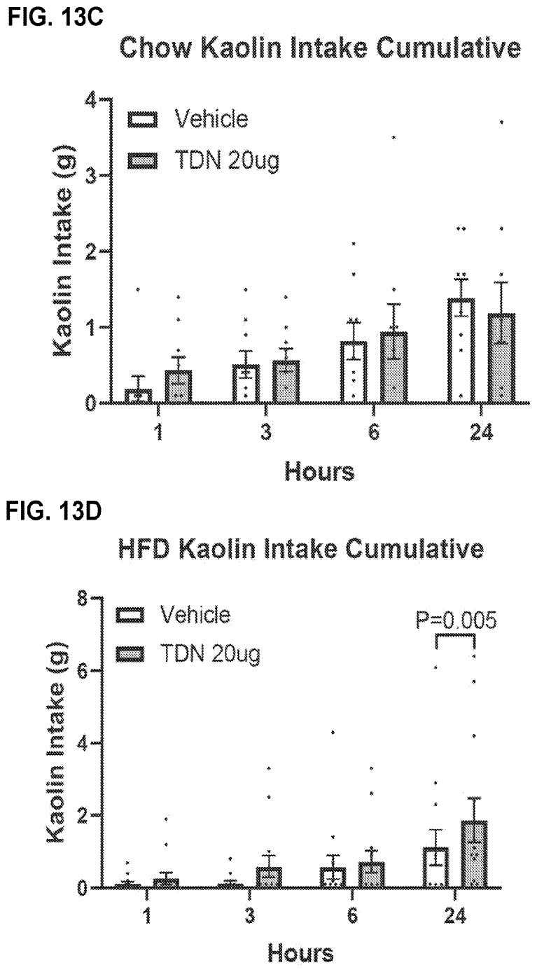

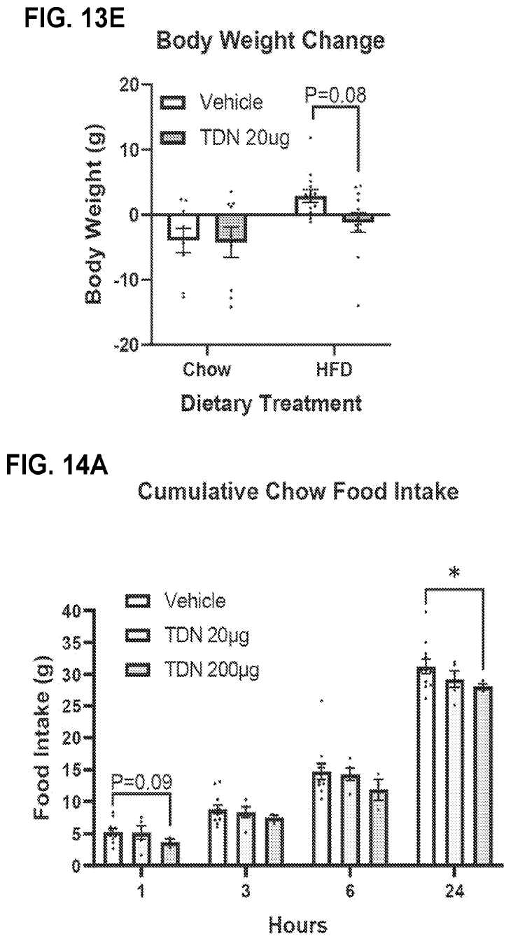

Tridecaneuropeptide (TDN) is a predicted cleavage product of ODN that when administered into the 4^{th} ventricle (20 g) did not suppress chow intake but did suppress HFD intake at 24 h post injections (FIGS. 13A-13B). TDN did not alter kaolin intake in chow rats but did mildly increase kaolin intake in HFD-maintained rats (FIGS. 13C-13D). 24 h body weight change tended to be reduced by TDN in HFD-maintained rats (FIG. 13E).

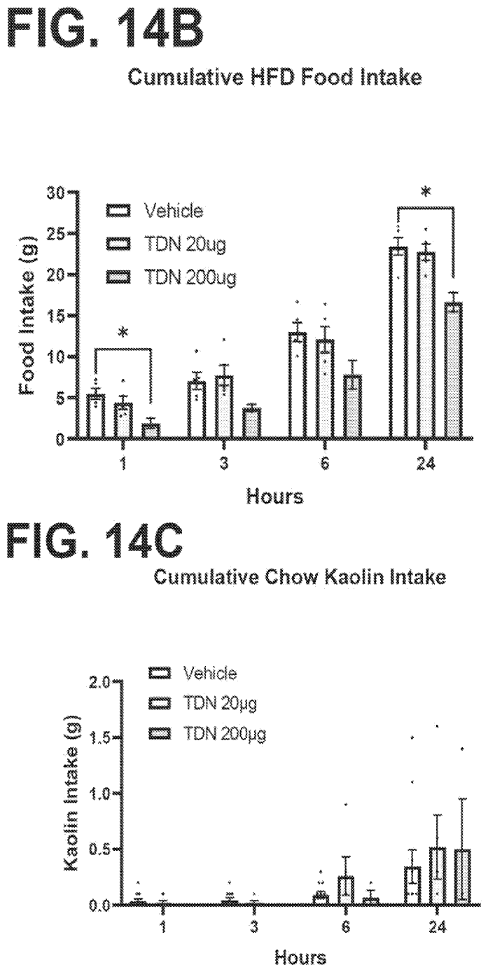

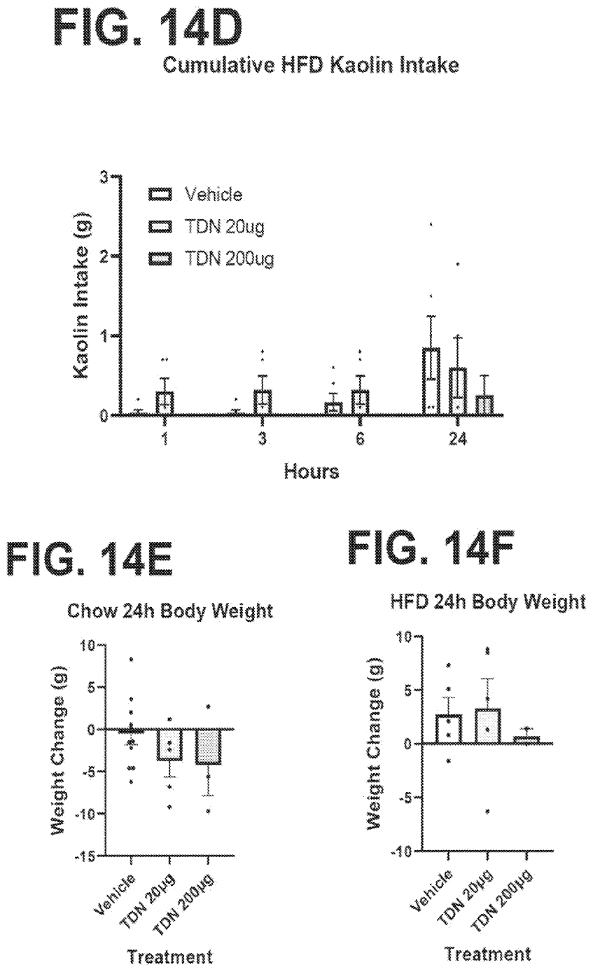

We next administered 20 and 200 μg TDN in the lateral ventricle of chow and HFD-maintained rats and observed that 200 μg TDN decreased 24 h chow intake and 1 and 24 h HFD intake (FIGS. 14A-14B). TDN did not affect kaolin intake or significantly change 24 h body weight in either diet group (FIGS. 14C-14F). FIGS. 13 and 14 demonstrate that central TDN also shows anorectic effects.

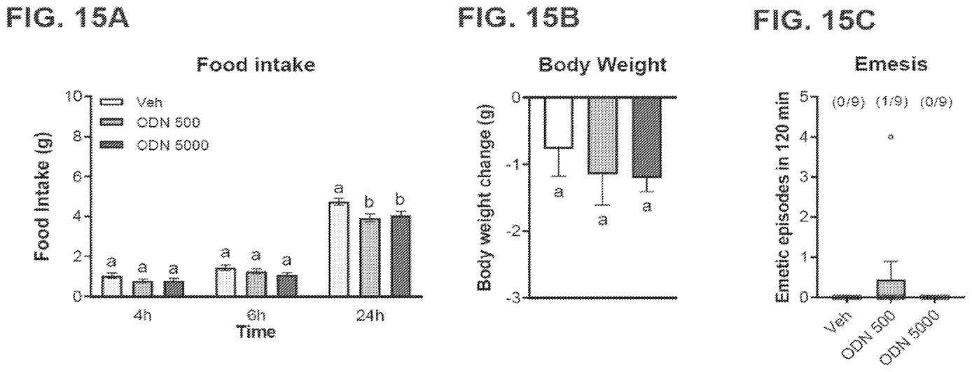

To investigate whether ODN has anorectic effects when administered intraperitoneally and to assess the emetic response to peripheral ODN in a vomiting model, we injected intraperitoneal ODN at 500 and 5000 μg in the shrew. Both doses of ODN suppressed 24 h food intake without significantly altering 24 h body weight, and only one of 9 shrews had an emetic episode to the low ODN dose, and no emetic episodes were observed with the high ODN dose (FIGS. 15A-15C).

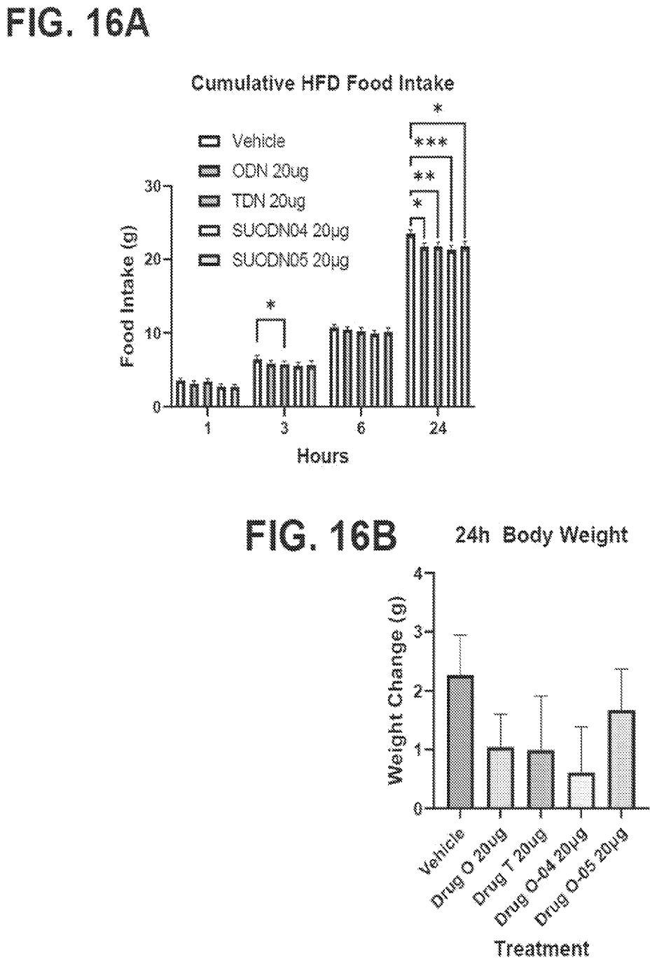

We tested the anorectic response to multiple novel ODN derivative peptides (ODN 20 μg, TDN 20 g, SUODN04 20 g, and SUODN05 20 μg) administered in the 4′ ventricle. All ODN based peptides suppressed 24 h HFD intake relative to vehicle and did not significantly alter 24 h body weight change (FIGS. 16A-16B).

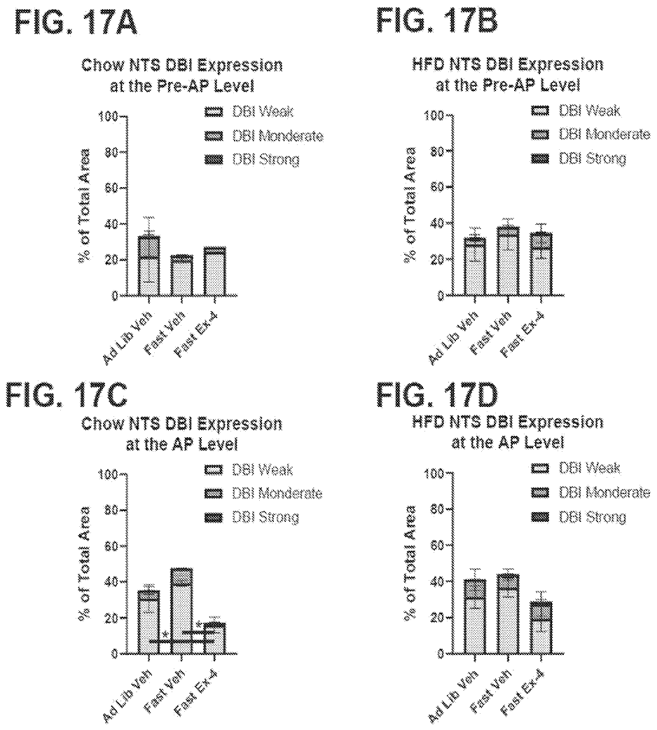

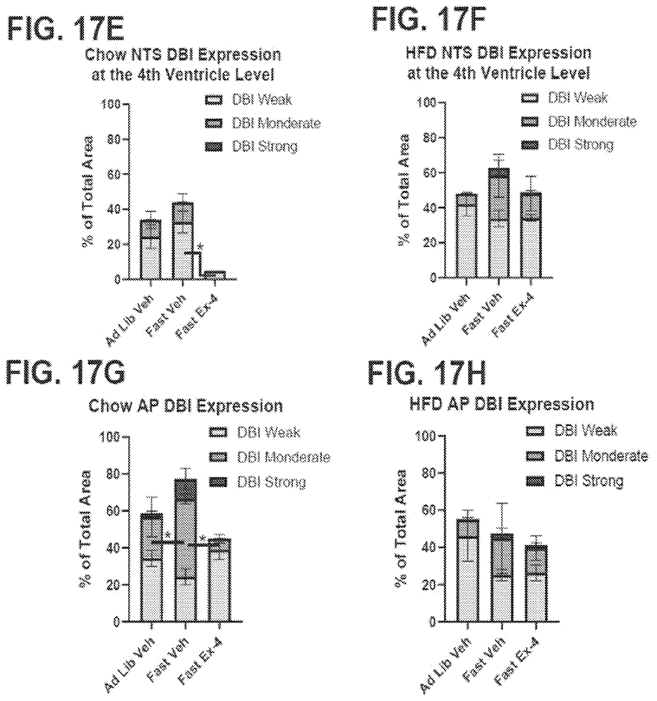

DBI is synthesized by populations of glial cells in the dorsal vagal complex (DVC) of the hindbrain which comprises the area postrema (AP) and nucleus tractus solitarius (NTS). To investigate where DVC DBI protein expression is regulated by nutritional state and GLP-1 receptor signaling, we quantified the amount of fluorescently labeled DBI protein in the AP and NTS in both chow and HFD-maintained rats that we ad libitum fed, 24 h fasted, or 24 h fasted plus a 4^{th} ventricle injection of the GLP-1 receptor agonist exendin-4 (Ex-4). At the pre-AP level, NTS DBI expression did not change with treatment in either chow or HFD-maintained rats (FIGS. 17A-17B). NTS DBI expression at the level of the AP was reduced by Ex-4 treatment compared to both fed and fasted groups in chow-maintained rats but was not different between treatments in HFD-maintained rats (FIGS. 17C-17D). NTS DBI expression at the 4^{th} ventricle level was reduced by Ex-4 treatment compared to the fasted group in chow-maintained rats but was not different between treatments in HFD-maintained rats (FIGS. 17E-17F). In the AP, DBI expression was increased with fasting and decreased with Ex-4 treatment in chow-maintained rats but not different between treatments in HFD-maintained rats (FIGS. 17G-17H). These data suggest that DBI protein expression in the NTS and AP is regulated by fasting and counter-regulated by Ex-4 treatment in the fasted state in chow but not HFD-maintained rats.

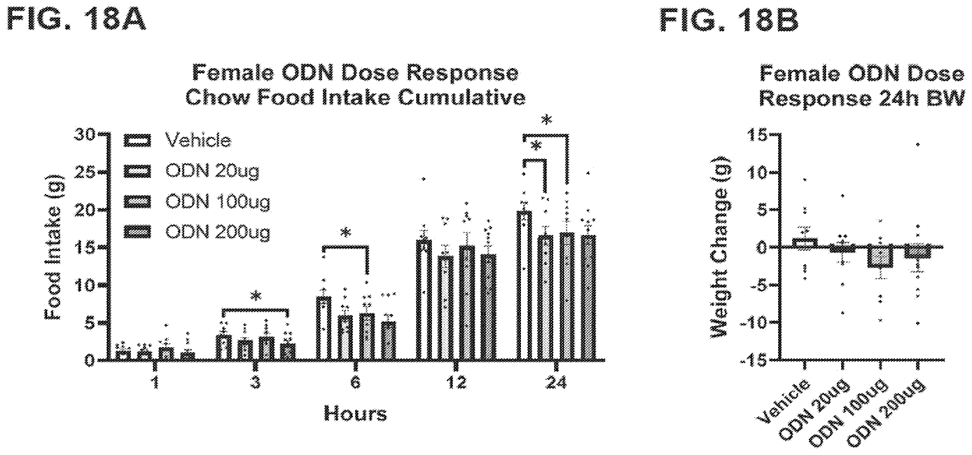

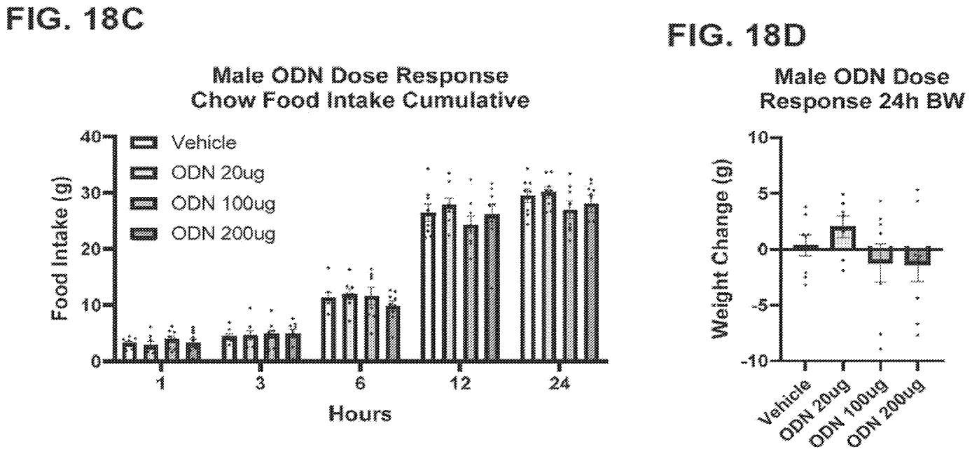

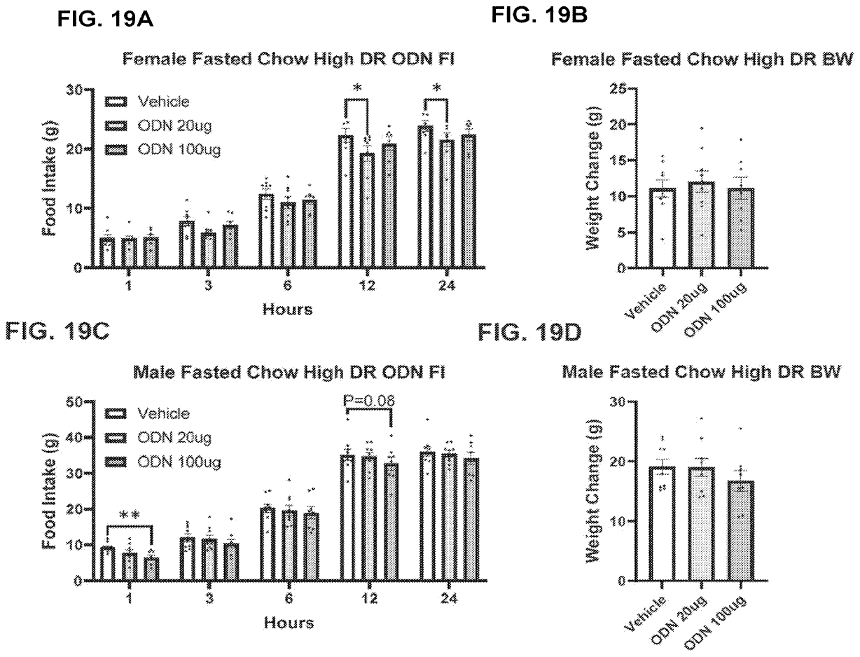

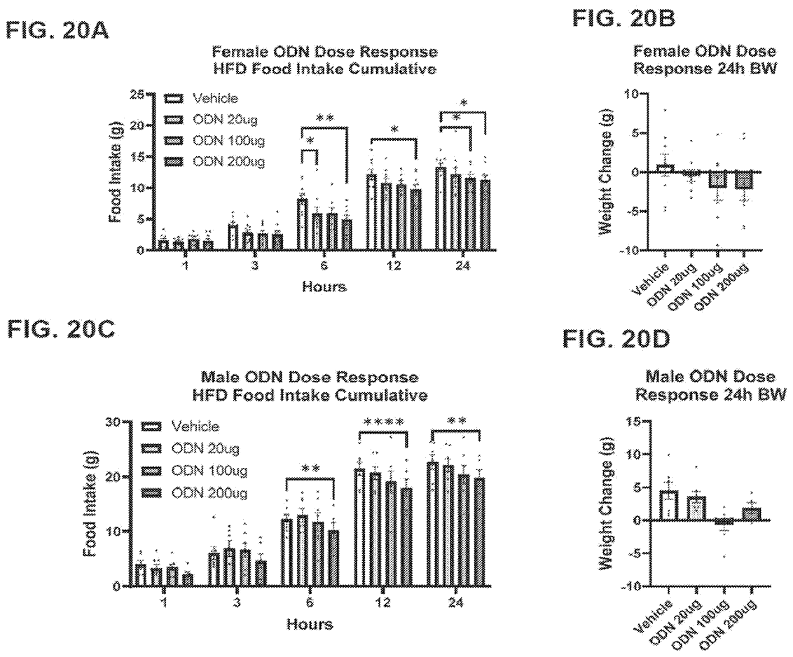

In additional studies, we observed that females are more sensitive to the hypophagic effects of exogenous lateral ventricle ODN than males on chow and HFD (Figures. 15-21). The effects of lateral ventricle ODN at increasing doses (20, 100, and 200 μg/2 μL) on 24 hour food intake on body weight and intake were compared in male and female rats. The results are shown in FIGS. 18A-18D. The data reveal that exogenous lateral ventricle ODN is less anorectic after an overnight fast in male and female rats on chow and HFD. Also see FIGS. 19A to 19D. FIG. 20 shows that central ODN suppresses food intake more robustly in HFD fed females than males. Effect of lateral ventricle ODN (20, 100, and 200 μg/2 μL) on 24 hour food intake on body weight on female and male rats. See FIGS. 20A-20D. FIG. 21 shows that central ODN weakly suppresses food intake in HFD fed overnight fasted males.

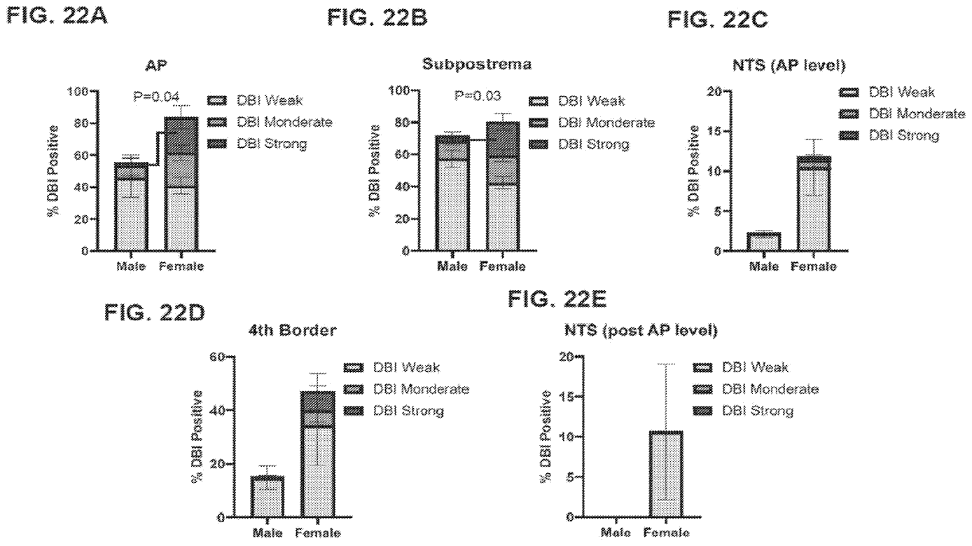

Females have higher endogenous hindbrain DBI protein expression as shown in FIG. 22. Brains from chow ad libitum fed male and female rats were collected and stained with a DBI antibody to detect DBI protein expression, fluorescence of which was quantified at low, moderate, and strong levels using HALO-AI software. DBI protein expression was determined in the AP (FIG. 22A), subpostrema border (FIG. 22B), NTS at the AP level (FIG. 22C), 4th ventricle border (FIG. 22D), and NTS at the post-AP level (FIG. 22E). We also determined the magnitude of DBI and Vimentin Co-Expression in Tanycyte Populated dorsal vagal complex (DVC) Areas. In the AP, 87.9% of vimentin positive cells co-express DBI and 16.9% of DBI positive cells co-express vimentin. In the subpostrema border between the AP and NTS, 74.8% of vimentin positive cells co-express DBI and 47.4% of DBI positive cells co-express vimentin. In the 4th ventricle border, 58.9% of vimentin positive cells co-express DBI and 81.5% of DBI positive cells co-express vimentin.

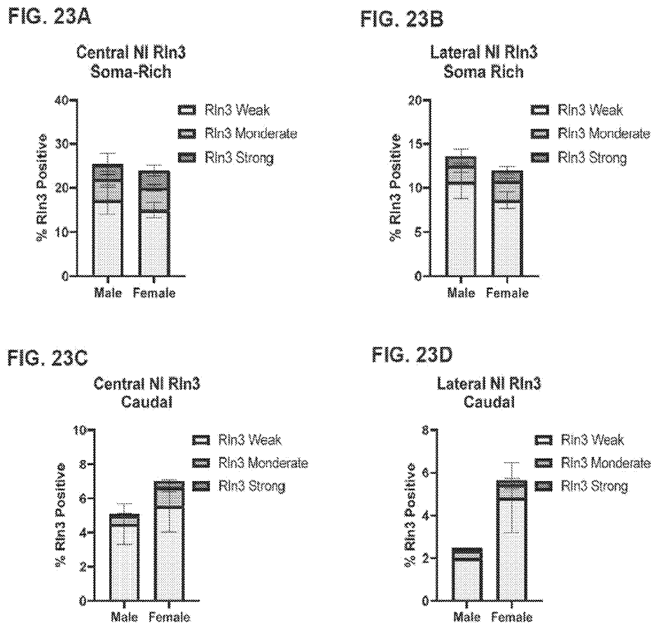

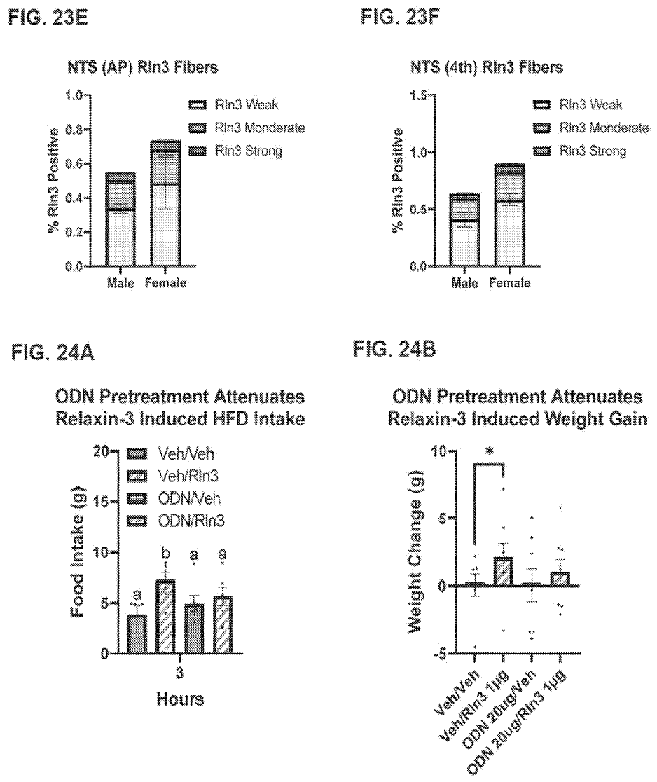

The data also show that females have higher endogenous relaxin-3 protein expression in nerve fibers. See FIG. 23. Brains from chow ad libitum fed male and female rats were collected and stained with a relaxin-3 (Rln3) antibody to detect Rln3 protein expression, fluorescence of which was quantified at low, moderate, and strong levels using HALO-AI software. Rln3 protein expression was determined in the central (FIG. 23A) and lateral (FIG. 23B) nucleus incertus rich with Rln3 positive neuron cell bodies, in the more caudal central (FIG. 23C) and lateral (FIG. 23D) nucleus incertus rich with Rln3 positive nerve fibers, and in the NTS at the AP level (FIG. 23E) and 4th ventricle level (FIG. 23F). We also observed that pretreatment with ODN attenuates central Relaxin-3 induced hyperphagia as shown in FIG. 24.

After chemical synthesis, the purity select peptides was determined by MALDI-ToF MS and RP-HPLC. The results are as follows: ODN [1911.13 g/mol; 11.521 min TR]; 100% pure, TDN [1454.63 μg/mol; 11.587 min TR]; 100% pure, OP [909.95 μg/mol; 5.017 min TR]; 96.4% pure, AntOP [909.95 μg/mol; 14.941 min TR]; 96% pure, and SUODN-03 [553.68 μg/mol; 11.372 min TR]; 95% pure. TR=retention time on Zorbax analytical C18 column.

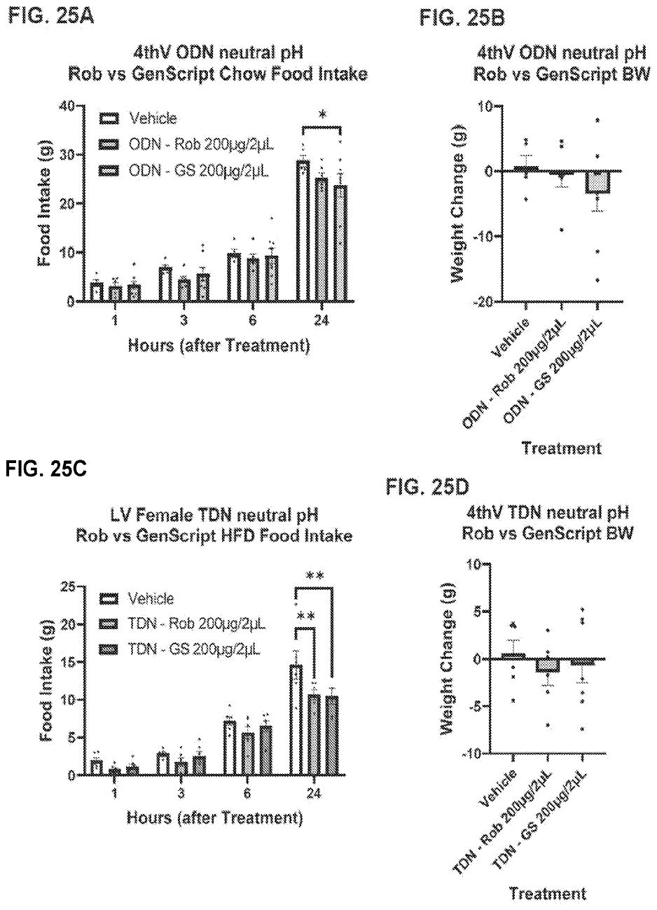

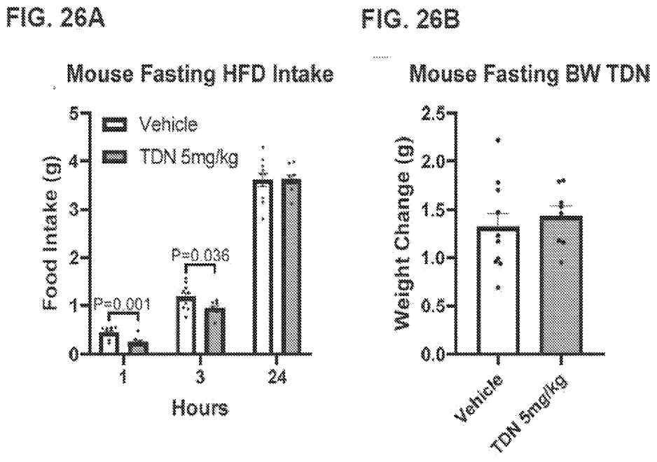

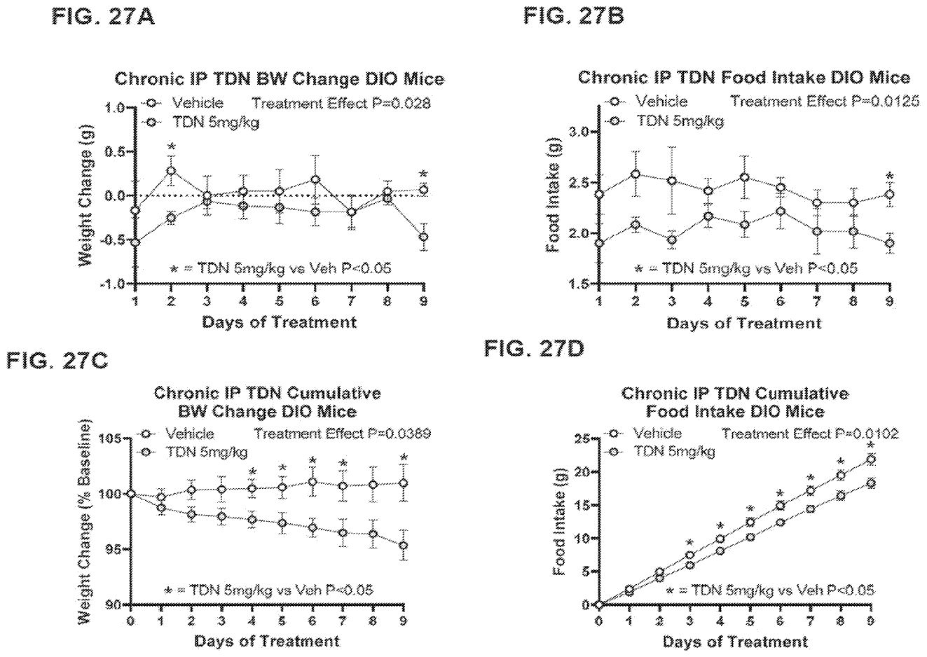

Central optimized ODN and TDN suppresses 24 h food intake in rats. ODN and TDN were optimized using an acetate salt precipitation, rather than a TFA salt precipitation to maintain a neutral >4.5 pH in HEPES buffer solution. The effect of 4th ventricle ODN (200 μg/2 μL) on 24 h food intake (FIG. 25A) and body weight (FIG. 25B) in male chow fed rats, and lateral ventricle TDN (200 μg/2 μL) on 24 h food intake (FIG. 25C) and body weight (FIG. 25D) in female HFD fed rats following treatments arc shown. Peripheral TDN suppresses fasting induced refeeding in DIO mice. See FIG. 26. The data also demonstrate that chronic peripheral TDN suppresses food intake in mice. Effect of daily IP TDN (5 mg/kg) on daily 24 h weight change, and food intake, cumulative weight change, and food intake over 9 days in male HFD fed mice following treatments are shown in FIG. 27.

In previous studies, we observed that central ODN signaling mediates hindbrain glucose sensing. 24-hour food intake, 1 hour blood glucose, and body weight change in chow fed rats pretreated 4th ventricle with an ODN agonist (OP 20 μg/2 μL) or vehicle followed by 4th ventricle treatment with 5-TG (210 μg/2 μL) or vehicle were determined and compared to 24-hour food intake, 1 hour blood glucose, and body weight change in chow fed rats pretreated 4th ventricle with an ODN antagonist (AntOP 100 μg/2 μL) or vehicle followed by 4th ventricle treatment with glucose (5.5 mM in 3 μL) or vehicle.

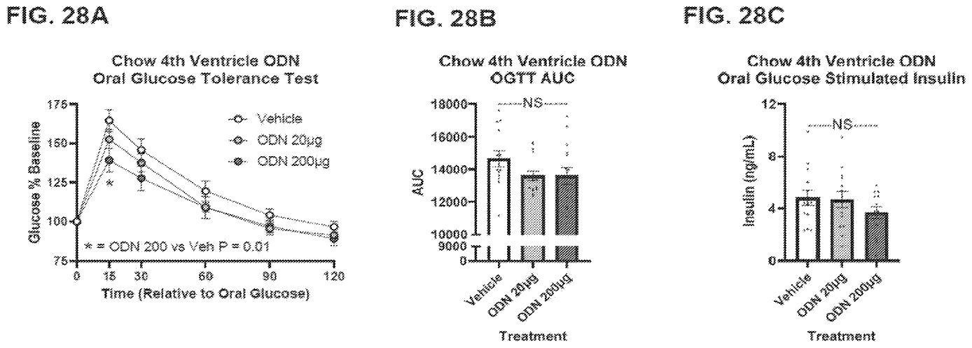

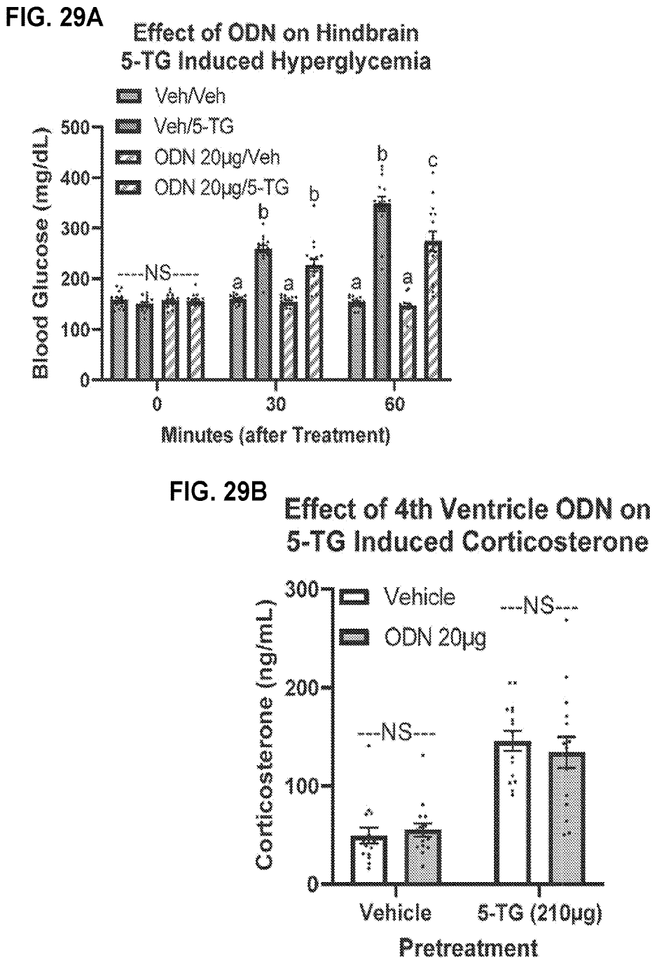

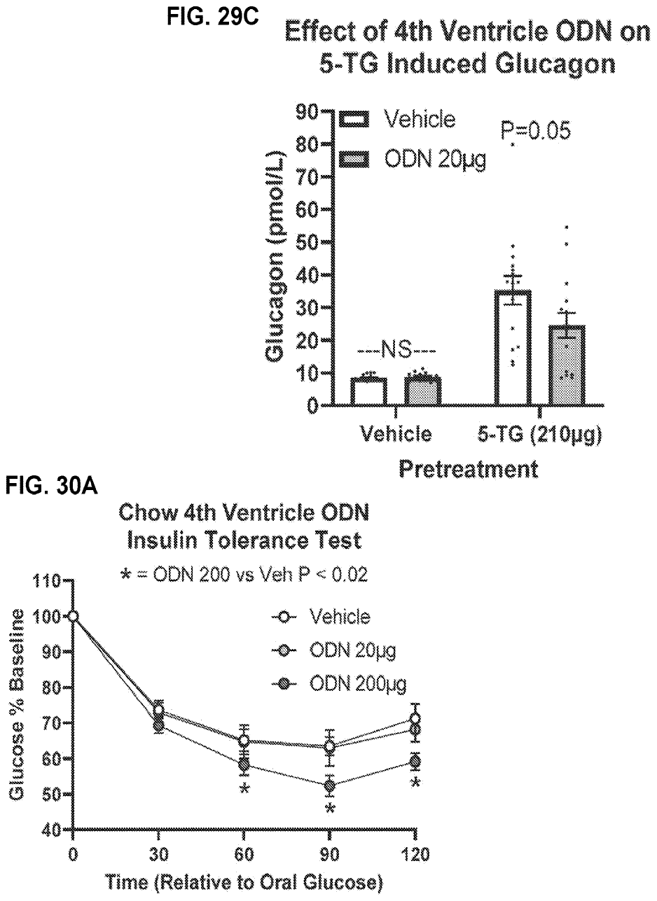

FIGS. 28A-28C show that hindbrain ODN improves glucose tolerance in chow rats. Hindbrain ODN signaling regulates the counterregulatory response. FIG. 29 are graphs showing the effects of administration to 4th ventricle with ODN (20 μg/2 μL) or vehicle followed by 4th ventricle treatment with 5-TG to indue hyperglycemia (210 μg/2 μL) or vehicle hour blood glucose, and 30-minute plasma corticosterone and plasma glucagon in chow fed rats.

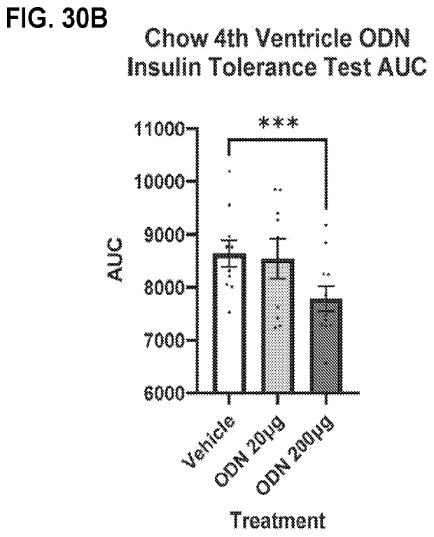

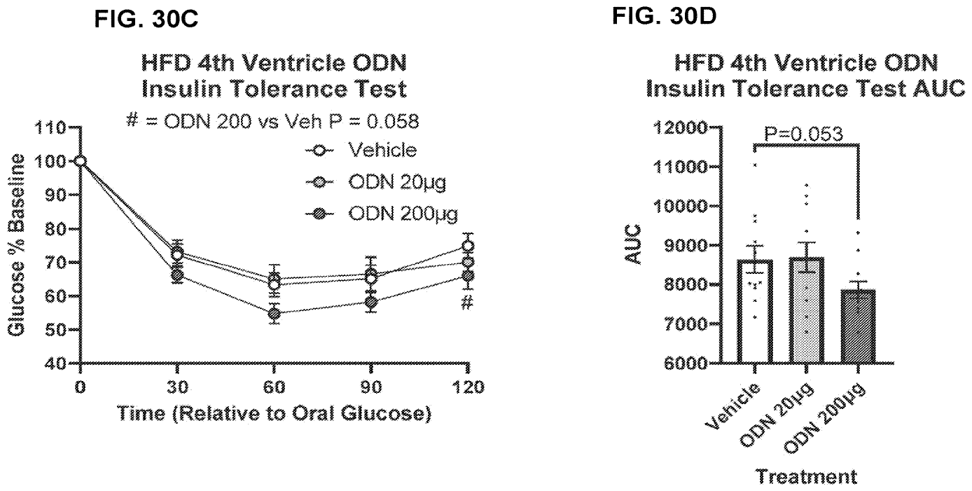

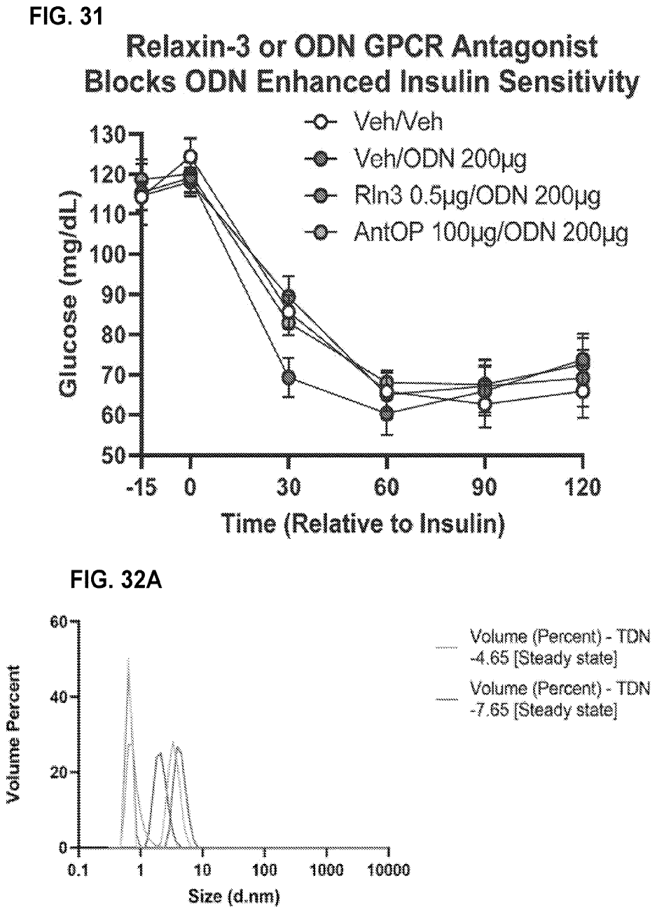

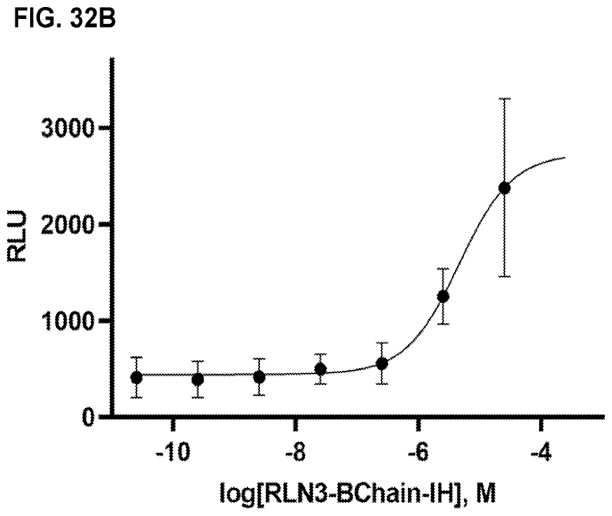

In additional studies, we observed that hindbrain ODN improves insulin tolerance in chow rats. See FIG. 30. Moreover, pretreatment with relaxin-3 and ODN antagonist block hindbrain ODN improvements in insulin tolerance in chow rats as shown in FIG. 31.

We also generated Dynamic light scattering data showing TDN has varying hydrodynamic radii (d. nm) supporting formation of complexing structures in solution (e.g., monomer, dimer, tetramer., etc) at different concentration. Such structures can exhibit activity and may advantageously interact with, or modulate, different functions at target receptor(s).

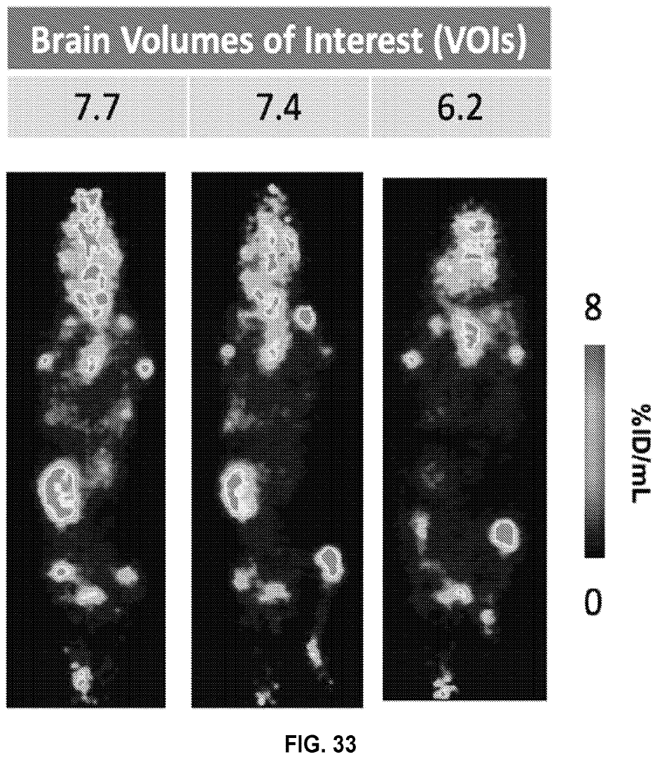

Finally, FIG. 33 ⁸⁹Zirconium labelled TDN PET scans in athymic nude mice (n=3) in coronal view demonstrate significant uptake in the brain. Insulin tolerance TDN-DFO (0.19 mg, 80 nmol) was reconstituted in saline (pH 7) and reacted with 89Zr (930 mCi, 34.4 MBq) in a 1:5 ratio (peptide:activity) for 45 minutes at RT. Radiochemical purity was assessed via instant thin-layer silica gel chromatography (iTLC-SG; Agilent Technologies) using 50 mM EDTA as the mobile phase. The reaction was quenched by adding EDTA (5 mL, 50 mM) to sequester unreacted activity, and the unbound radiometal was removed by centrifugation via spin column (MWCO=10 kDa) with sterile saline as the solvent. Athymic nude mice (n=3) were injected (i.v.) with [89Zr]-DFO-TDN in sterile saline (4.8-5.6 MBq, 130-150 μCi, 60-70 nmol) in the lateral tail vein. Small-animal PET was conducted 48 hours post-injection using a Bruker Albira Si PET/CT while the mice were anesthetized with 2% isoflurane. Images were reconstructed through maximum likelihood expectation maximization in 12 iterations and 0.75 mm voxel resolution and were analyzed using PMOD version 4.3 software. Volumes of interest were measured manually by drawing on the target site across various planar sections and expressed as percent injected dose per volume of tissue (% ID/mL).

In total, these results demonstrate that central ODN and novel ODN based peptides (e.g., TDN) decrease food intake in chow and HFD-maintained rats and mice, that hindbrain DBI protein expression is regulated by nutritional state and GLP-1 agonism in chow rats is blunted in HFD-fed rats, that central GLP-1 agonism upregulates DBI mRNA expression in chow-fed rats, that blocking ODN signaling with either an antibody targeting DBI or an ODN antagonist attenuates the anorectic effect of central and peripheral GLP-1 analogues, that ODN and GLP-1 signaling are cooperative to suppress food intake, and that ODN is involved in the hindbrain glucose sensing response.

### Example 2

### Screening for Druggable Targets Using gpcrMAX and orphanMAX Assays

The gpcrMAX and orphanMAX panels are intended to provide a cost-effective means at identifying possible interactions with a selection of known GPCR or orphan GPCR targets. Compounds are typically tested at a single concentration and as a result provides a semi-quantitative determination of efficacy. Any potential interactions can be confirmed in a follow-up dose response.

gpcrMAX—Agonist Mode

Activation of GPCR by a compound acting as an agonist will result in an increase in beta-arrestin recruitment to the target GPCR. The result tables (Tables 4 and 5) provide the mean, standard deviation (SD) and % CV for control values for both baseline (Control 1) and maximal control ligand response (Control 2—Max). Compound RLU (Raw values) are provided for the test compound plus the mean RLU, standard deviation and % CV. Compound % Activity is calculated as the % activity relative to the baseline (0% activity) and Max (100% activity) values.

gpcrMAX—Antagonist Mode

Inhibition of GPCR activation by a compound acting as an antagonist of ligand binding will result in a decrease in beta-arrestin recruitment to the target GPCR. The result table provides mean, standard deviation (SD) and % CV for control values for both EC80 (Control 1) and basal ligand response (Control 2—Basal). Compound RLU (Raw values) are provided for the test compound plus the mean RLU, standard deviation and % CV. Compound % Inhibition is calculated as the % inhibition relative to the EC80 (0% inhibition) and basal (100% inhibition) values. These assays provide a means to determine if compound activity observed in the panels is potentially significant and worthy of follow-up. Different approaches are recommended for each panel type and assay mode.

orphanMAX—Agonist Mode

Activation of Orphan GPCR by a compound acting as an agonist will result in an increase in beta-arrestin recruitment to the target orphan GPCR. The result table provides the mean, standard deviation (SD) and % CV for control value for baseline activity observed. Since different GPCRs exhibit varying levels of expression and constitutive arrestin recruitment, the Baseline Mean RLU value will differ from target to target. Compound RLU (Raw values) are provided for the test compound plus the mean RLU, standard deviation and % CV. Since a known ligand is typically not available for orphan GPCR assays, % activity is calculated differently for the orphanMAX GPCR panel compared to the gpcrMAX panel. Activity is calculated relative to the baseline response only. Therefore a 2-fold increase in compound Mean RLU over baseline will generate a % Activity value of 100%. Likewise, 3 fold increase is equal to 200%.

Each of the assays can be perform on GPCR having known or unknown ligands.

The neuropeptide relaxin-3 is synthesized in the nucleus incertus of the hindbrain and has orexigenic effects to increase food intake and promote weight gain. Independent GPCR screening performed by Eurofins identified the glial synthesized satiation factor ODN as an endogenous antagonist of the relaxin-3 receptor. To test the functional role of ODN to antagonize the effects of relaxin-3 in vivo, we injected rats into the nucleus incertus, a nucleus that not only synthesizes relaxin-3 but expressed a high level of the relaxin-3 receptor, with ODN prior to relaxin-3. As expected, relaxin-3 increased 24-hour food intake and body weight, and impressively pretreatment with ODN reversed these effects (FIG. 31). These data support that ODN is the endogenous antagonist of the relaxin-3 receptor, rxfp3, and provides a mechanism for the anorectic effects of central ODN signaling.

TABLE 4
Control Dose response curves for selected GPCR Biosensor Assays

| Compound | Assay | Result | RC50 | | Curve | Curve | Max | |
|---|---|---|---|---|---|---|---|---|
| Name* | Target** | Type | (μM) | Hill | Bottom | Top | Response | EC80(uM) |
| PACAP-27 | ADCYAP1R1 | EC50 | 0.00391138 | 1.6617 | 4.247 | 98.885 | 100.67 | 0.011803144 |
| 2-CI-IB-MECA | ADORA3 | EC50 | 0.01525879 | 1.1017 | −4.5785 | 100.04 | 107.6 | 0.053418022 |
| Phenylephrine | ADRA1B | EC50 | 0.1628722 | 1.1119 | −0.1258 | 99.546 | 102.6 | 0.390036549 |
| UK 14,304 | ADRA2A | EC50 | 0.006584155 | 0.67942 | 0 | 104 | 103.78 | 0.024991519 |
| UK 14,304 | ADRA2B | EC50 | 0.1715043 | 1.5427 | −3.5776 | 99.007 | 101.76 | 0.537999516 |
| UK 14,304 | ADRA2C | EC50 | 0.0528202 | 1.3001 | −14.689 | 103.6 | 115.02 | 0.300840457 |
| Isoproterenol | ADRB1 | EC50 | 0.03615513 | 0.9032 | −1.565 | 98.992 | 103.08 | 0.209926871 |
| Isoproterenol | ADRB2 | EC50 | 0.01980067 | 1.3003 | 0.016719 | 95.144 | 95.309 | 0.100758445 |
| Angiotensin II | AGTR1 | EC50 | 0.000440941 | 1.2265 | −1.5534 | 98.855 | 106.42 | 0.001548612 |
| Apelin-13 | AGTRL1 | EC50 | 0.00125317 | 1.1108 | −5.3927 | 96.774 | 101.08 | 0.002314048 |
| Vasopressin | AVPR1A | EC50 | 0.004293781 | 0.85042 | 0 | 99.755 | 103.94 | 0.009484267 |
| Vasopressin | AVPR1B | EC50 | 0.001541452 | 0.89493 | −4.8549 | 97.66 | 99.458 | 0.002748026 |
| Vasopressin | AVPR2 | EC50 | 0.000656432 | 1.6806 | −3.3253 | 101.69 | 104.04 | 0.001253359 |
| LDA-Bradykinin | BDKRB1 | EC50 | 0.003652918 | 0.98709 | −2.8311 | 99.753 | 103.16 | 0.014021861 |
| Bradykinin | BDKRB2 | EC50 | 0.005572115 | 1.3847 | 0.091551 | 105.59 | 103.91 | 0.009824615 |
| TAPN-Bombesin | BRS3 | EC50 | 0.001243827 | 0.79106 | −8 | 91.941 | 100.84 | 0.0037314 |
| C3A Receptor | C3AR1 | EC50 | 0.1870269 | 1.2488 | 0.69156 | 102.72 | 100.98 | 0.457270729 |
| Agonist | | | | | | | | |
| Complement C5a | C5aR1 | EC50 | 0.001378066 | 1.5463 | 0.41487 | 100.88 | 102.6 | 0.003635129 |
| Complement C5a | C5L2 | EC50 | 0.001570543 | 1.1743 | −9.3389 | 101.65 | 101.89 | 0.002700941 |
| Calcitonin | CALCR | EC50 | 0.03093926 | 0.98215 | 2.5604 | 105 | 102.54 | 0.07287192 |
| beta CGRP | CALCRL- | EC50 | 0.002395687 | 1.7485 | 3.2986 | 98.905 | 99.214 | 0.011737397 |
| | RAMP1 | | | | | | | |
| Adrenomedullin | CALCRL- | EC50 | 0.000999747 | 1.7618 | 2.1065 | 101.76 | 101.61 | 0.002285984 |
| | RAMP2 | | | | | | | |
| Adrenomedullin | CALCRL- | EC50 | 0.002876935 | 1.3768 | 5.154 | 100.06 | 103.15 | 0.005843889 |
| | RAMP3 | | | | | | | |
| Calcitonin | CALCR- | EC50 | 0.01643236 | 0.89546 | −4.3244 | 107.25 | 108.07 | 0.033690535 |
| | RAMP2 | | | | | | | |
| Calcitonin | CALCR- | EC50 | 0.1433346 | 0.72186 | −0.87325 | 108 | 103.06 | 0.383951451 |
| | RAMP3 | | | | | | | |
| CCK-8 | CCKAR | EC50 | 0.003881683 | 1.1405 | −2.2947 | 100 | 101.65 | 0.013860384 |
| CCK-8 | CCKBR | EC50 | 0.000357388 | 1.5527 | −1.718 | 102.31 | 101.37 | 0.00082258 |
| CCL3 | CCR1 | EC50 | 0.001041598 | 1.1108 | −10 | 93.69 | 104.89 | 0.002529119 |
| CCL27 | CCR10 | EC50 | 0.02626274 | 1.4073 | 1.8602 | 100 | 100.93 | 0.109379657 |
| CCL2 | CCR2 | EC50 | 0.002631843 | 1.0598 | −1.4626 | 100 | 100.4 | 0.006923753 |
| CCL13 | CCR3 | EC50 | 0.04062094 | 0.83007 | 1.5876 | 105 | 100 | 0.139617612 |
| CCL22 | CCR4 | EC50 | 0.003437795 | 0.71432 | 0 | 102.68 | 101.2 | 0.019726878 |
| CCL3 | CCR5 | EC50 | 0.007553182 | 1.0897 | 1.515 | 100 | 100.91 | 0.030256773 |
| CCL20 | CCR6 | EC50 | 0.002950529 | 1.1253 | −4.4514 | 100 | 100.8 | 0.014296434 |
| CCL19 | CCR7 | EC50 | 0.005646118 | 2.036 | −1.7698 | 100.44 | 102.04 | 0.018964175 |
| CCL1 | CCR8 | EC50 | 0.0458469 | 1.3048 | 2.0288 | 100 | 104.05 | 0.159211768 |
| CCL25 | CCR9 | EC50 | 0.172951 | 1.5366 | 0.38159 | 100 | 100 | 0.37 |
| Acetylcholine | CHRM1 | EC50 | 1.402265 | 0.64719 | 0 | 106.04 | 100.58 | 6.956689738 |
| Acetylcholine | CHRM2 | EC50 | 2.755396 | 1.3779 | 2.6926 | 100 | 100.81 | 15.79076552 |
| Acetylcholine | CHRM3 | EC50 | 0.1457965 | 0.56689 | −12.236 | 108.49 | 100 | 0.396098916 |
| Acetylcholine | CHRM4 | EC50 | 1.900527 | 1.1007 | −6.8542 | 100.27 | 104.44 | 5.7015 |
| Acetylcholine | CHRM5 | EC50 | 0.05513787 | 1.2059 | −0.59006 | 100.43 | 101.2 | 0.314649976 |
| Chemerin | CMKLR1 | EC50 | 0.001004986 | 1.7846 | −1.3765 | 100 | 104.08 | 0.004813577 |
| CP55940 | CNR1 | EC50 | 0.004738708 | 0.91424 | −6.794 | 101.26 | 104.27 | 0.020427882 |
| CP55940 | CNR2 | EC50 | 0.002215185 | 2.9877 | 0.44915 | 95.673 | 99.599 | 0.00348976 |
| Sauvagine | CRHR1 | EC50 | 0.003279745 | 1.8277 | −1.8621 | 100 | 98.203 | 0.010328697 |
| Sauvagine | CRHR2 | EC50 | 0.005993492 | 1.4129 | 0.49814 | 100 | 100.67 | 0.024510765 |
| PGD2 | CRTH2 | EC50 | 0.003340105 | 0.72799 | −6.8088 | 100.58 | 100 | 0.009117293 |
| Fractalkine | CX3CR1 | EC50 | 0.000444457 | 1.1237 | −5.4026 | 100 | 105.09 | 0.001447817 |
| CXCL8 | CXCR1 | EC50 | 0.002795374 | 0.97352 | −4.9222 | 100 | 100.67 | 0.014839994 |
| CXCL8 | CXCR2 | EC50 | 0.000303753 | 0.98092 | 0 | 98.662 | 106.94 | 0.000967833 |
| CXCL11 | CXCR3 | EC50 | 0.0120304 | 1.0922 | 3.2275 | 100 | 106.47 | 0.017871267 |
| CXCL12 | CXCR4 | EC50 | 0.00354797 | 0.93797 | 11.901 | 100 | 104.76 | 0.015607547 |
| CXCL13 | CXCR5 | EC50 | 0.02095897 | 1.4103 | 3.2801 | 100 | 100.33 | 0.061247455 |
| CXCL16 | CXCR6 | EC50 | 0.006805656 | 1.5222 | 1.7888 | 105 | 104.92 | 0.020964889 |
| CXCL12 | CXCR7 | EC50 | 0.008011062 | 1.6149 | −1.7859 | 100 | 104.6 | 0.038326347 |
| Dopamine | DRD1 | EC50 | 0.3960719 | 1.3107 | 1.162 | 100 | 105.47 | 1.395694261 |
| Dopamine | DRD2L | EC50 | 0.1377704 | 1.1993 | −1.5603 | 104.48 | 101.1 | 0.3 |
| Dopamine | DRD2S | EC50 | 0.0496947 | 1.0934 | −2.5746 | 96.826 | 101.97 | 0.1 |
| Dopamine | DRD3 | EC50 | 0.002081086 | 1.5067 | −6.7782 | 101.49 | 100.91 | 0.006941111 |
| Dopamine | DRD4 | EC50 | 0.03668766 | 1.2385 | −4.5543 | 101.36 | 103.84 | 0.11 |
| Dopamine | DRD5 | EC50 | 0.03291091 | 1.0774 | −0.024319 | 101.92 | 102.54 | 0.129197003 |
| 7a,25- | EBI2 | EC50 | 0.003632537 | 1.1467 | 0.71485 | 103.06 | 101.26 | 0.019217982 |
| Dihydroxycholesterol | | | | | | | | |
| S-1-P | EDG1 | EC50 | 0.01898279 | 1.1089 | −2.857 | 100.31 | 102.86 | 0.056949 |
| S-1-P | EDG3 | EC50 | 0.01059547 | 1.1291 | −3.5563 | 97.815 | 100.81 | 0.041142218 |
| Oleoyl LPA | EDG4 | EC50 | 1.802619 | 1.3783 | 1.2853 | 93.075 | 105.68 | 2.985698189 |
| S-1-P | EDG5 | EC50 | 0.03718605 | 1.7 | 1.1708 | 96.946 | 98.798 | 0.121076002 |
| S-1-P | EDG6 | EC50 | 0.03051476 | 0.94885 | −5 | 98.769 | 101.46 | 0.174604601 |
| Oleoyl LPA | EDG7 | EC50 | 0.1738741 | 1.1261 | 1.7177 | 98.075 | 100.16 | 0.396950027 |
| Endothelin I | EDNRA | EC50 | 0.002385338 | 1.1297 | −1.6519 | 95.562 | 104.63 | 0.009867465 |
| Endothelin 3 | EDNRB | EC50 | 0.006583851 | 0.97303 | −5.299 | 96.586 | 102.05 | 0.020798812 |
| TFLLR-NH2 | F2R | EC50 | 14.49439 | 1.2839 | 0.6282 | 100 | 103.36 | 28.70048374 |
| SLIGRL-NH2 | F2RL1 | EC50 | 0.5519605 | 1.1076 | −2.2601 | 101.71 | 101.05 | 2.133539975 |
| AYPGKF-NH2 | F2RL3 | EC50 | 2.282086 | 1.9026 | −2.8765 | 101.99 | 101.77 | 9.09924257 |
| GW9508 | FFAR1 | EC50 | 0.9588609 | 1.0269 | 1.9496 | 101.83 | 103.4 | 1.860194065 |
| WKYMVm-NH2 | FPR1 | EC50 | 0.004949029 | 1.96 | −1.2578 | 101.3 | 98.917 | 0.025202958 |
| WKYMVm-NH2 | FPRL1 | EC50 | 0.002164662 | 2.1405 | 2.3178 | 101.26 | 103.5 | 0.011786601 |
| FSH | FSHR | EC50 | 0.007459333 | 1.0758 | 0.71439 | 110 | 114.13 | 0.01221875 |
| Galanin | GALR1 | EC50 | 0.004739363 | 2.0213 | −0.48036 | 102.15 | 104.23 | 0.011069657 |
| Galanin | GALR2 | EC50 | 0.006805342 | 1.5902 | 7.6827 | 102.82 | 100.83 | 0.012196885 |
| Glucagon | GCGR | EC50 | 0.002334533 | 1.9316 | 3.6064 | 102.29 | 103.15 | 0.009837639 |
| Ghrelin | GHSR | EC50 | 0.008482619 | 2.4678 | 0.62296 | 94.252 | 98.147 | 0.017292727 |
| GIP | GIPR | EC50 | 0.004871469 | 1.215 | 2.1835 | 106.8 | 110.21 | 0.018519859 |
| Exendin-4 | GLP1R | EC50 | 0.005824921 | 1.2562 | −0.9728 | 98.597 | 101.42 | 0.012641739 |
| GLP II (1-33) | GLP2R | EC50 | 0.002297417 | 0.78513 | 7.6211 | 102.59 | 100.69 | 0.008969645 |
| Chemerin | GPR1 | EC50 | 0.002258396 | 1.3937 | −0.015963 | 98.668 | 99.696 | 0.004668147 |
| QRFP-26 | GPR103 | EC50 | 0.009995395 | 0.98753 | −7.5636 | 103.59 | 100.28 | 0.027112934 |
| Nicotinic Acid | GPR109A | EC50 | 3.644005 | 1.1508 | 1.8885 | 108 | 107.49 | 5.717675529 |
| 3-Hydroxyoctanoic | GPR109B | EC50 | 372.9495 | 1.3226 | 1.0862 | 105 | 100 | 1121.048501 |
| Acid | | | | | | | | |
| Oleoyl | GPR119 | EC50 | 1.838176 | 0.96535 | −12.768 | 105.21 | 103.94 | 8.990611912 |
| Ethanolamide | | | | | | | | |
| GW9508 | GPR120 | EC50 | 6.96835 | 1.0467 | −7.1742 | 113.42 | 106.32 | 22.92817458 |
| Zaprinast | GPR35 | EC50 | 1.740095 | 0.65872 | −4.2289 | 105.27 | 101.96 | 3.647777388 |
| Oleoyl LPA | GPR92 | EC50 | 0.7273777 | 0.82717 | 1.1979 | 100.49 | 101.14 | 1.439294801 |
| GRP | GRPR | EC50 | 0.001622146 | 1.687 | 0.94583 | 93.998 | 99.427 | 0.005916924 |
| Orexin A | HCRTR1 | EC50 | 0.004212985 | 1.8912 | −0.62218 | 96.031 | 98.192 | 0.010713082 |
| Orexin A | HCRTR2 | EC50 | 0.01824904 | 1.7906 | 0.17334 | 102.47 | 100.46 | 0.042508285 |
| Histamine | HRH1 | EC50 | 0.04169174 | 1.0719 | 0 | 91.374 | 101.92 | 0.212258123 |
| Histamine | HRH2 | EC50 | 4.489645 | 0.8807 | 4.9249 | 100 | 101.25 | 10.76974282 |
| R-a | HRH3 | EC50 | 0.1068533 | 1.1514 | −5.4023 | 100.87 | 100.75 | 0.46650708 |
| methylhistamine | | | | | | | | |
| Histamine | HRH4 | EC50 | 0.04382884 | 0.98004 | −2.1869 | 102.92 | 104.69 | 0.103737387 |
| Serotonin/5-HT | HTR1A | EC50 | 0.04870012 | 1.5705 | −4.0678 | 101.04 | 101.92 | 0.254448746 |
| Serotonin/5-HT | HTR1B | EC50 | 0.03952119 | 1.1066 | −2.5647 | 101.29 | 102.29 | 0.174110069 |
| Serotonin/5-HT | HTR1E | EC50 | 0.003518195 | 1.5289 | −7.3023 | 105.51 | 101.99 | 0.012 |
| Serotonin/5-HT | HTR1F | EC50 | 0.03048808 | 0.93606 | −10.321 | 108.78 | 107.92 | 0.104806427 |
| Serotonin/5-HT | HTR2A | EC50 | 0.02168602 | 0.96597 | 0.085792 | 99.25 | 99.349 | 0.097704961 |
| Serotonin/5-HT | HTR2C | EC50 | 0.001968656 | 1.1706 | −5.4272 | 95.557 | 92.1 | 0.010286228 |
| Serotonin/5-HT | HTR5A | EC50 | 0.00576647 | 1.4028 | 4.5741 | 104.79 | 100.52 | 0.026434531 |
| Kisspeptin-10 | KISS1R | EC50 | 0.0176153 | 1.2385 | −0.57157 | 104.88 | 107.69 | 0.037598975 |
| hCG | LHCGR | EC50 | 0.002273421 | 0.65664 | −6.0082 | 112.86 | 107.66 | 0.00575213 |
| Leukotriene B4 | LTB4R | EC50 | 0.1410552 | 0.68356 | 4.511 | 110 | 122.03 | 0.404785512 |
| Melanotan II | MC1R | EC50 | 0.000436958 | 1.2155 | 4.2998 | 100 | 100.11 | 0.001887071 |
| Melanotan II | MC3R | EC50 | 0.000935923 | 1.2752 | 4.9004 | 100 | 100.58 | 0.003000514 |
| Melanotan II | MC4R | EC50 | 0.001266689 | 0.90099 | −5.252 | 99.724 | 100.46 | 0.003259382 |
| Melanotan II | MC5R | EC50 | 0.007774995 | 0.7756 | −6.5723 | 104.01 | 107.84 | 0.023310177 |
| MCH | MCHR1 | EC50 | 0.1674376 | 1.4457 | 1.9607 | 100 | 100 | 0.443766951 |
| MCH | MCHR2 | EC50 | 0.005514293 | 1.5346 | 3.1744 | 99.371 | 103.44 | 0.020247081 |
| Motilin | MLNR | EC50 | 0.001590376 | 0.80863 | −9.2463 | 100 | 102.1 | 0.005626276 |
| BAM(8-22) | MRGPRX1 | EC50 | 3.504257 | 1.2114 | 3.3193 | 100 | 105.15 | 8.528562388 |
| Cortistatin 14 | MRGPRX2 | EC50 | 0.1553549 | 0.83242 | −0.33236 | 100 | 112.59 | 0.728551508 |
| 2-Iodomelatonin | MTNR1A | EC50 | 0.000730298 | 1.5901 | −0.91382 | 100.7 | 102.67 | 0.002769725 |
| Neuromedin B | NMBR | EC50 | 0.000575586 | 1.2642 | 0 | 97.735 | 101.96 | 0.003317285 |
| Neuromedin U-25 | NMU1R | EC50 | 0.001429816 | 1.6529 | −0.055343 | 101.34 | 101.18 | 0.003675309 |
| Neuropeptide W23 | NPBWR1 | EC50 | 0.002169053 | 1.7253 | 6.5309 | 98.703 | 100.19 | 0.008007844 |
| Neuropeptide W23 | NPBWR2 | EC50 | 0.003615681 | 2.3725 | −0.83886 | 99.167 | 101.21 | 0.012247634 |
| RFRP-3 | NPFFR1 | EC50 | 0.02515244 | 0.84424 | −4.7513 | 100.95 | 100.6 | 0.130008768 |
| Neuropeptide S | NPSR1B | EC50 | 0.02360206 | 0.95946 | −5.6163 | 97.744 | 106.99 | 0.04538166 |
| Peptide YY | NPY1R | EC50 | 0.003925817 | 0.85992 | −9.7065 | 100 | 100.86 | 0.012422491 |
| Peptide YY | NPY2R | EC50 | 0.002585294 | 2.1905 | −0.53426 | 100 | 103.69 | 0.005199872 |
| [Lys 8,9] | NTSR1 | EC50 | 0.000237744 | 1.8392 | 4.3056 | 105.37 | 101.31 | 0.001184194 |
| Neurotensin | | | | | | | | |
| DADLE | OPRD1 | EC50 | 0.004407782 | 0.73776 | −5.8906 | 102.31 | 106.58 | 0.007547797 |
| Dynorphin A | OPRK1 | EC50 | 0.08292394 | 0.91322 | −2.6726 | 103.11 | 101.11 | 0.255636022 |
| Orphanin FQ | OPRL1 | EC50 | 0.006485448 | 1.0555 | −2.9695 | 101.29 | 103.92 | 0.017256278 |
| [Met] | OPRM1 | EC50 | 0.5881959 | 1.1453 | −2.0822 | 98.969 | 100.17 | 2.114225824 |
| Enkephalin | | | | | | | | |
| 5-OxoETE | OXER1 | EC50 | 1.143734 | 0.75922 | −0.2145 | 100 | 100 | 6 |
| Oxytocin | OXTR | EC50 | 0.003228495 | 0.76819 | −1.7706 | 102.14 | 95.386 | 0.007191409 |
| 2-methylthio-ADP | P2RY1 | EC50 | 0.02037793 | 0.92597 | −0.53627 | 99.336 | 100.4 | 0.041089184 |
| ATP | P2RY11 | EC50 | 371.0395 | 4.523 | 1.8016 | 104.83 | 103.71 | 1301.314611 |
| 2-methylthio-ADP | P2RY12 | EC50 | 0.002716565 | 1.0334 | −5.0447 | 101 | 102.97 | 0.0045 |
| UTP | P2RY2 | EC50 | 0.4311327 | 1.9756 | 2.6252 | 96.948 | 105.35 | 2.522195098 |
| UTP | P2RY4 | EC50 | 0.1264911 | 0.93915 | −5 | 98.026 | 101.62 | 0.37947 |
| UDP | P2RY6 | EC50 | 0.01326906 | 0.83577 | −10.471 | 102.75 | 100.95 | 0.074875901 |
| Pancreatic | PPYR1 | EC50 | 0.001504423 | 1.2096 | −1.6292 | 97.898 | 96.839 | 0.005004449 |
| Polypeptide | | | | | | | | |
| PrRP-31 | PRLHR | EC50 | 0.002440338 | 1.5554 | −0.29601 | 99.754 | 101.19 | 0.010716143 |
| EG VEGF | PROKR1 | EC50 | 0.01765347 | 1.0565 | −0.62347 | 106.22 | 102.59 | 0.053731645 |
| EG VEGF | PROKR2 | EC50 | 0.01061989 | 1.2011 | −1.0624 | 102.93 | 104.29 | 0.03003967 |
| PAF | PTAFR | EC50 | 0.003823139 | 1.8262 | 0.53715 | 100 | 100.46 | 0.017847207 |
| Prostaglandin E2 | PTGER2 | EC50 | 0.6873754 | 0.84772 | −1.8177 | 99.103 | 99.417 | 1.216458735 |
| Prostaglandin E2 | PTGER3 | EC50 | 0.003761144 | 1.0648 | −4.5647 | 98.536 | 101.62 | 0.017821547 |
| Prostaglandin E2 | PTGER4 | EC50 | 0.000552344 | 1.8119 | −2.9138 | 102.66 | 103.34 | 0.00119196 |
| Cloprostenol | PTGFR | EC50 | 0.007043037 | 0.95661 | −0.16663 | 95.972 | 100.52 | 0.034523733 |
| Beraprost | PTGIR | EC50 | 0.2187656 | 1.3158 | 8.5689 | 100 | 102.4 | 0.842518747 |
| PTH(1-34) | PTHR1 | EC50 | 0.000873333 | 3.3619 | 3.9763 | 98.042 | 96.306 | 0.002358215 |
| TIP-39 | PTHR2 | EC50 | 0.00148146 | 1.9233 | 2.2565 | 100.48 | 101.68 | 0.004810385 |
| Relaxin-3 | RXFP3 | EC50 | 0.01188393 | 0.83341 | −2.1488 | 100 | 103.3 | 0.062548114 |
| Secretin | SCTR | EC50 | 0.001738682 | 2.4137 | −0.52064 | 99.971 | 103.23 | 0.009198736 |
| Somatostatin 28 | SSTR1 | EC50 | 0.005890877 | 0.9221 | −0.029622 | 102.68 | 102.65 | 0.020335557 |
| Somatostatin 28 | SSTR2 | EC50 | 0.004881576 | 1.1232 | −1.9751 | 99.89 | 107.78 | 0.008191175 |
| Tyr-SST 14 | SSTR3 | EC50 | 0.0321048 | 1.0935 | 1.3337 | 100 | 100 | 0.054536886 |
| Somatostatin 28 | SSTR5 | EC50 | 0.007112627 | 1.4958 | 3.8156 | 100.7 | 102.87 | 0.014065586 |
| Substance P | TACR1 | EC50 | 0.002450811 | 1.6523 | 1.016 | 100.41 | 101.11 | 0.011067561 |
| Substance P | TACR2 | EC50 | 0.08746414 | 0.67952 | 0 | 100 | 108.43 | 0.263941019 |
| Substance P | TACR3 | EC50 | 0.01315104 | 0.99897 | 0 | 99.743 | 102.24 | 0.026872641 |
| I-BOP | TBXA2R | EC50 | 0.02125299 | 1.1152 | −2.3978 | 100 | 103.12 | 0.086206858 |
| TRH | TRHR | EC50 | 0.002020041 | 1.4836 | −1.4107 | 100 | 102.21 | 0.007135007 |
| TSH | TSHR(L) | EC50 | 0.04951309 | 0.82938 | −0.47128 | 111.68 | 105.96 | 0.087896021 |
| Urotensin II | UTR2 | EC50 | 0.001186397 | 1.0063 | 0 | 98.712 | 108.04 | 0.004221852 |
| VIP | VIPR1 | EC50 | 0.001727508 | 1.8414 | 5.9378 | 101.12 | 102.57 | 0.003693381 |
| VIP | VIPR2 | EC50 | 0.006734727 | 3.1618 | 1.3517 | 101.54 | 101.68 | 0.012686961 |

*The assay mode for each compound is Agonist
**The assay name for each compound is Arrestin

TABLE 5
Compound agonist and antagonist activity determined us GPCR Biosensor Assays

| Compound | GPCR | Assay | | Rep 1 | Rep 2 | Mean | | % |
|---|---|---|---|---|---|---|---|---|
| ID | ID | Mode | Conc(uM) | RLU | RLU | RLU | SD | Activity |
| SUODN | ADCYAP1R1 | Antagonist | 2.5 | 319800 | 307880 | 313840.00 | 8428.7 | 0.9 |
| SUODN | ADORA3 | Antagonist | 2.5 | 40040 | 42880 | 41460.00 | 2008.2 | 0.5 |
| SUODN | ADRA1B | Antagonist | 2.5 | 74080 | 76960 | 75520.00 | 2036.5 | 0.1 |
| SUODN | ADRA2A | Antagonist | 2.5 | 114520 | 124560 | 119540.00 | 7099.4 | −27.2 |
| SUODN | ADRA2B | Antagonist | 2.5 | 59920 | 58240 | 59080.00 | 1187.9 | −36.8 |
| SUODN | ADRA2C | Antagonist | 2.5 | 65360 | 66760 | 66060.00 | 990 | −26.4 |
| SUODN | ADRB1 | Antagonist | 2.5 | 72600 | 89400 | 81000.00 | 11879.4 | 12.6 |
| SUODN | ADRB2 | Antagonist | 2.5 | 63640 | 59960 | 61800.00 | 2602.2 | −11.7 |
| SUODN | AGTR1 | Antagonist | 2.5 | 73920 | 82880 | 78400.00 | 6335.7 | −8.5 |
| SUODN | AGTRL1 | Antagonist | 2.5 | 183000 | 190920 | 186960.00 | 5600.3 | −23.4 |
| SUODN | AVPR1A | Antagonist | 2.5 | 66880 | 68400 | 67640.00 | 1074.8 | −2.4 |
| SUODN | AVPR1B | Antagonist | 2.5 | 16160 | 15960 | 16060.00 | 141.4 | −25.7 |
| SUODN | AVPR2 | Antagonist | 2.5 | 271080 | 258240 | 264660.00 | 9079.2 | −11.2 |
| SUODN | BDKRB1 | Antagonist | 2.5 | 15000 | 14960 | 14980.00 | 28.3 | −5.7 |
| SUODN | BDKRB2 | Antagonist | 2.5 | 289280 | 280360 | 284820.00 | 6307.4 | −1.7 |
| SUODN | BRS3 | Antagonist | 2.5 | 229520 | 204560 | 217040.00 | 17649.4 | −5.8 |
| SUODN | C3AR1 | Antagonist | 2.5 | 97320 | 100080 | 98700.00 | 1951.6 | 4 |
| SUODN | C5aR1 | Antagonist | 2.5 | 228600 | 227760 | 228180.00 | 594 | −14.2 |
| SUODN | C5L2 | Antagonist | 2.5 | 112800 | 128400 | 120600.00 | 11030.9 | 3.5 |
| SUODN | CALCR | Antagonist | 2.5 | 24800 | 21720 | 23260.00 | 2177.9 | −22.3 |
| SUODN | CALCRL- | Antagonist | 2.5 | 47120 | 52200 | 49660.00 | 3592.1 | −19 |
| | RAMP1 | | | | | | | |
| SUODN | CALCRL- | Antagonist | 2.5 | 258960 | 257480 | 258220.00 | 1046.5 | −11.6 |
| | RAMP2 | | | | | | | |
| SUODN | CALCRL- | Antagonist | 2.5 | 134000 | 143120 | 138560.00 | 6448.8 | −3.5 |
| | RAMP3 | | | | | | | |
| SUODN | CALCR- | Antagonist | 2.5 | 53400 | 54240 | 53820.00 | 594 | −17.8 |
| | RAMP2 | | | | | | | |
| SUODN | CALCR- | Antagonist | 2.5 | 10400 | 10720 | 10560.00 | 226.3 | −0.1 |
| | RAMP3 | | | | | | | |
| SUODN | CCKAR | Antagonist | 2.5 | 95280 | 105120 | 100200.00 | 6957.9 | −17.2 |
| SUODN | CCKBR | Antagonist | 2.5 | 385280 | 359760 | 372520.00 | 18045.4 | −7.8 |
| SUODN | CCR1 | Antagonist | 2.5 | 364000 | 336280 | 350140.00 | 19601 | −5.4 |
| SUODN | CCR10 | Antagonist | 2.5 | 38360 | 39520 | 38940.00 | 820.2 | −16.6 |
| SUODN | CCR2 | Antagonist | 2.5 | 142120 | 162680 | 152400.00 | 14538.1 | −15.4 |
| SUODN | CCR3 | Antagonist | 2.5 | 81520 | 83600 | 82560.00 | 1470.8 | −5.8 |
| SUODN | CCR4 | Antagonist | 2.5 | 304760 | 313960 | 309360.00 | 6505.4 | 1.3 |
| SUODN | CCR5 | Antagonist | 2.5 | 163480 | 173360 | 168420.00 | 6986.2 | −16.1 |
| SUODN | CCR6 | Antagonist | 2.5 | 246240 | 230080 | 238160.00 | 11426.8 | −14.8 |
| SUODN | CCR7 | Antagonist | 2.5 | 860640 | 822960 | 841800.00 | 26643.8 | −19.2 |
| SUODN | CCR8 | Antagonist | 2.5 | 1337080 | 1294280 | 1315680.00 | 30264.2 | −17 |
| SUODN | CCR9 | Antagonist | 2.5 | 2642000 | 2928040 | 2785020.00 | 202260.8 | −10.1 |
| SUODN | CHRM1 | Antagonist | 2.5 | 52600 | 47360 | 49980.00 | 3705.2 | 9.3 |
| SUODN | CHRM2 | Antagonist | 2.5 | 68400 | 68520 | 68460.00 | 84.8 | 3.3 |
| SUODN | CHRM3 | Antagonist | 2.5 | 33440 | 31760 | 32600.00 | 1187.9 | 12.6 |
| SUODN | CHRM4 | Antagonist | 2.5 | 140200 | 154880 | 147540.00 | 10380.3 | 5.8 |
| SUODN | CHRM5 | Antagonist | 2.5 | 391840 | 395640 | 393740.00 | 2687 | −1.5 |
| SUODN | CMKLR1 | Antagonist | 2.5 | 3476520 | 3565600 | 3521060.00 | 62989.1 | −18.3 |
| SUODN | CNR1 | Antagonist | 2.5 | 64440 | 62800 | 63620.00 | 1159.7 | −37.5 |
| SUODN | CNR2 | Antagonist | 2.5 | 85560 | 76000 | 80780.00 | 6759.9 | 3.6 |
| SUODN | CRHR1 | Antagonist | 2.5 | 416760 | 489360 | 453060.00 | 51336 | 5.4 |
| SUODN | CRHR2 | Antagonist | 2.5 | 253720 | 298600 | 276160.00 | 31735 | −8.6 |
| SUODN | CRTH2 | Antagonist | 2.5 | 56200 | 53400 | 54800.00 | 1979.9 | 0.6 |
| SUODN | CX3CR1 | Antagonist | 2.5 | 5791720 | 6074960 | 5933340.00 | 200280.9 | −0.8 |
| SUODN | CXCR1 | Antagonist | 2.5 | 1235560 | 1690880 | 1463220.00 | 321959.9 | 10.2 |
| SUODN | CXCR2 | Antagonist | 2.5 | 671720 | 727920 | 699820.00 | 39739.4 | −9.8 |
| SUODN | CXCR3 | Antagonist | 2.5 | 1269680 | 1238920 | 1254300.00 | 21750.6 | −7.5 |
| SUODN | CXCR4 | Antagonist | 2.5 | 15800 | 13680 | 14740.00 | 1499.1 | −28.5 |
| SUODN | CXCR5 | Antagonist | 2.5 | 672240 | 666520 | 669380.00 | 4044.6 | −18.9 |
| SUODN | CXCR6 | Antagonist | 2.5 | 6040 | 6240 | 6140.00 | 141.4 | −1.3 |
| SUODN | CXCR7 | Antagonist | 2.5 | 2353280 | 2751880 | 2552580.00 | 281852.8 | −9.6 |
| SUODN | DRD1 | Antagonist | 2.5 | 31160 | 28680 | 29920.00 | 1753.6 | 5.7 |
| SUODN | DRD2L | Antagonist | 2.5 | 24440 | 23560 | 24000.00 | 622.2 | −16.8 |
| SUODN | DRD2S | Antagonist | 2.5 | 167880 | 163960 | 165920.00 | 2771.9 | −13.4 |
| SUODN | DRD3 | Antagonist | 2.5 | 91680 | 86680 | 89180.00 | 3535.5 | −1 |
| SUODN | DRD4 | Antagonist | 2.5 | 6120 | 6400 | 6260.00 | 198 | 2.5 |
| SUODN | DRD5 | Antagonist | 2.5 | 34240 | 34920 | 34580.00 | 480.8 | 4.2 |
| SUODN | EBI2 | Antagonist | 2.5 | 459960 | 392240 | 426100.00 | 47885.3 | −27.3 |
| SUODN | EDG1 | Antagonist | 2.5 | 175200 | 199560 | 187380.00 | 17225.1 | −25.9 |
| SUODN | EDG3 | Antagonist | 2.5 | 365840 | 496760 | 431300.00 | 92574.4 | 15.4 |
| SUODN | EDG4 | Antagonist | 2.5 | 83080 | 79840 | 81460.00 | 2291 | 4.6 |
| SUODN | EDG5 | Antagonist | 2.5 | 186160 | 194720 | 190440.00 | 6052.8 | −11.2 |
| SUODN | EDG6 | Antagonist | 2.5 | 359400 | 376760 | 368080.00 | 12275.4 | 4.5 |
| SUODN | EDG7 | Antagonist | 2.5 | 95800 | 97720 | 96760.00 | 1357.6 | 5 |
| SUODN | EDNRA | Antagonist | 2.5 | 46000 | 49080 | 47540.00 | 2177.9 | 2.5 |
| SUODN | EDNRB | Antagonist | 2.5 | 58040 | 53520 | 55780.00 | 3196.1 | −9.9 |
| SUODN | F2R | Antagonist | 2.5 | 36440 | 44800 | 40620.00 | 5911.4 | −17 |
| SUODN | F2RL1 | Antagonist | 2.5 | 282720 | 263680 | 273200.00 | 13463.3 | −9.2 |
| SUODN | F2RL3 | Antagonist | 2.5 | 444280 | 402360 | 423320.00 | 29641.9 | −25.9 |
| SUODN | FFAR1 | Antagonist | 2.5 | 111920 | 105840 | 108880.00 | 4299.2 | −3.9 |
| SUODN | FPR1 | Antagonist | 2.5 | 384840 | 369200 | 377020.00 | 11059.2 | −11.1 |
| SUODN | FPRL1 | Antagonist | 2.5 | 193080 | 196400 | 194740.00 | 2347.6 | −17.7 |
| SUODN | FSHR | Antagonist | 2.5 | 23840 | 21880 | 22860.00 | 1385.9 | −16.9 |
| SUODN | GALR1 | Antagonist | 2.5 | 381800 | 474280 | 428040.00 | 65393.2 | −22.5 |
| SUODN | GALR2 | Antagonist | 2.5 | 474160 | 412960 | 443560.00 | 43274.9 | −29.7 |
| SUODN | GCGR | Antagonist | 2.5 | 138720 | 143080 | 140900.00 | 3083 | −17.2 |
| SUODN | GHSR | Antagonist | 2.5 | 162640 | 137200 | 149920.00 | 17988.8 | −7.4 |
| SUODN | GIPR | Antagonist | 2.5 | 13680 | 14160 | 13920.00 | 339.4 | −18.9 |
| SUODN | GLP1R | Antagonist | 2.5 | 173160 | 164680 | 168920.00 | 5996.3 | −11 |
| SUODN | GLP2R | Antagonist | 2.5 | 58800 | 57040 | 57920.00 | 1244.5 | −15.4 |
| SUODN | GPR1 | Antagonist | 2.5 | 118880 | 110160 | 114520.00 | 6166 | −1.3 |
| SUODN | GPR103 | Antagonist | 2.5 | 24560 | 23920 | 24240.00 | 452.6 | 14.6 |
| SUODN | GPR109A | Antagonist | 2.5 | 107680 | 105360 | 106520.00 | 1640.5 | −16 |
| SUODN | GPR109B | Antagonist | 2.5 | 433560 | 449200 | 441380.00 | 11059.2 | −17 |
| SUODN | GPR119 | Antagonist | 2.5 | 57680 | 55320 | 56500.00 | 1668.8 | −1 |
| SUODN | GPR120 | Antagonist | 2.5 | 12160 | 14000 | 13080.00 | 1301.1 | 22.7 |
| SUODN | GPR35 | Antagonist | 2.5 | 183880 | 167160 | 175520.00 | 11822.8 | −12.2 |
| SUODN | GPR92 | Antagonist | 2.5 | 111400 | 109720 | 110560.00 | 1187.9 | 10.6 |
| SUODN | GRPR | Antagonist | 2.5 | 118560 | 109520 | 114040.00 | 6392.2 | −12.2 |
| SUODN | HCRTR1 | Antagonist | 2.5 | 326440 | 320840 | 323640.00 | 3959.8 | −1.7 |
| SUODN | HCRTR2 | Antagonist | 2.5 | 285920 | 279520 | 282720.00 | 4525.5 | −2.7 |
| SUODN | HRH1 | Antagonist | 2.5 | 135360 | 118960 | 127160.00 | 11596.6 | −38.5 |
| SUODN | HRH2 | Antagonist | 2.5 | 36840 | 31440 | 34140.00 | 3818.4 | −13.3 |
| SUODN | HRH3 | Antagonist | 2.5 | 12280 | 12240 | 12260.00 | 28.3 | −8.6 |
| SUODN | HRH4 | Antagonist | 2.5 | 3240 | 3040 | 3140.00 | 141.4 | 5.2 |
| SUODN | HTR1A | Antagonist | 2.5 | 320840 | 334600 | 327720.00 | 9729.8 | −6.2 |
| SUODN | HTR1B | Antagonist | 2.5 | 194200 | 164960 | 179580.00 | 20675.8 | −11.9 |
| SUODN | HTR1E | Antagonist | 2.5 | 3640 | 3640 | 3640.00 | 0 | −3.8 |
| SUODN | HTR1F | Antagonist | 2.5 | 65080 | 62160 | 63620.00 | 2064.8 | −23.3 |
| SUODN | HTR2A | Antagonist | 2.5 | 238960 | 219800 | 229380.00 | 13548.2 | −9.7 |
| SUODN | HTR2C | Antagonist | 2.5 | 166680 | 155720 | 161200.00 | 7749.9 | 2.2 |
| SUODN | HTR5A | Antagonist | 2.5 | 515760 | 512760 | 514260.00 | 2121.3 | −0.3 |
| SUODN | KISS1R | Antagonist | 2.5 | 26000 | 22480 | 24240.00 | 2489 | 13.2 |
| SUODN | LHCGR | Antagonist | 2.5 | 13280 | 14520 | 13900.00 | 876.8 | 7.2 |
| SUODN | LTB4R | Antagonist | 2.5 | 107160 | 106480 | 106820.00 | 480.8 | 10.3 |
| SUODN | MC1R | Antagonist | 2.5 | 17760 | 17960 | 17860.00 | 141.4 | 0.4 |
| SUODN | MC3R | Antagonist | 2.5 | 5400 | 5200 | 5300.00 | 141.4 | −2.1 |
| SUODN | MC4R | Antagonist | 2.5 | 14720 | 14160 | 14440.00 | 396 | −4.6 |
| SUODN | MC5R | Antagonist | 2.5 | 22560 | 18880 | 20720.00 | 2602.2 | 5.9 |
| SUODN | MCHR1 | Antagonist | 2.5 | 6840 | 6600 | 6720.00 | 169.7 | 5.1 |
| SUODN | MCHR2 | Antagonist | 2.5 | 36520 | 32280 | 34400.00 | 2998.1 | −10.5 |
| SUODN | MLNR | Antagonist | 2.5 | 94320 | 93560 | 93940.00 | 537.4 | −5.5 |
| SUODN | MRGPRX1 | Antagonist | 2.5 | 222520 | 199640 | 211080.00 | 16178.6 | 3.8 |
| SUODN | MRGPRX2 | Antagonist | 2.5 | 210400 | 221720 | 216060.00 | 8004.4 | −23.5 |
| SUODN | MTNR1A | Antagonist | 2.5 | 15480 | 15200 | 15340.00 | 198 | 2.2 |
| SUODN | NMBR | Antagonist | 2.5 | 121880 | 111440 | 116660.00 | 7382.2 | −7.7 |
| SUODN | NMU1R | Antagonist | 2.5 | 121560 | 105000 | 113280.00 | 11709.7 | −34.2 |
| SUODN | NPBWR1 | Antagonist | 2.5 | 84640 | 84680 | 84660.00 | 28.3 | −15 |
| SUODN | NPBWR2 | Antagonist | 2.5 | 139040 | 136760 | 137900.00 | 1612.2 | −1.1 |
| SUODN | NPFFR1 | Antagonist | 2.5 | 33920 | 37760 | 35840.00 | 2715.3 | −4.2 |
| SUODN | NPSR1B | Antagonist | 2.5 | 6520 | 6920 | 6720.00 | 282.8 | 15.5 |
| SUODN | NPY1R | Antagonist | 2.5 | 39200 | 42080 | 40640.00 | 2036.5 | −2.7 |
| SUODN | NPY2R | Antagonist | 2.5 | 346520 | 314120 | 330320.00 | 22910.3 | −10.3 |
| SUODN | NTSR1 | Antagonist | 2.5 | 267280 | 230400 | 248840.00 | 26078.1 | −21.4 |
| SUODN | OPRD1 | Antagonist | 2.5 | 36720 | 39520 | 38120.00 | 1979.9 | 10 |
| SUODN | OPRK1 | Antagonist | 2.5 | 6720 | 6320 | 6520.00 | 282.8 | −4.5 |
| SUODN | OPRL1 | Antagonist | 2.5 | 142080 | 137080 | 139580.00 | 3535.5 | −5.6 |
| SUODN | OPRM1 | Antagonist | 2.5 | 188640 | 187280 | 187960.00 | 961.7 | −21.1 |
| SUODN | OXER1 | Antagonist | 2.5 | 37440 | 40400 | 38920.00 | 2093 | −39.7 |
| SUODN | OXTR | Antagonist | 2.5 | 64080 | 67120 | 65600.00 | 2149.6 | 1.3 |
| SUODN | P2RY1 | Antagonist | 2.5 | 82720 | 80560 | 81640.00 | 1527.4 | −6.4 |
| SUODN | P2RY11 | Antagonist | 2.5 | 40480 | 41000 | 40740.00 | 367.7 | −5.6 |
| SUODN | P2RY12 | Antagonist | 2.5 | 104360 | 98520 | 101440.00 | 4129.5 | −11 |
| SUODN | P2RY2 | Antagonist | 2.5 | 107080 | 107520 | 107300.00 | 311.1 | −5.2 |
| SUODN | P2RY4 | Antagonist | 2.5 | 160480 | 142400 | 151440.00 | 12784.5 | 12.9 |
| SUODN | P2RY6 | Antagonist | 2.5 | 237200 | 228560 | 232880.00 | 6109.4 | −15.1 |
| SUODN | PPYR1 | Antagonist | 2.5 | 85600 | 86280 | 85940.00 | 480.8 | −11.4 |
| SUODN | PRLHR | Antagonist | 2.5 | 16560 | 15560 | 16060.00 | 707.1 | −22.2 |
| SUODN | PROKR1 | Antagonist | 2.5 | 44200 | 46760 | 45480.00 | 1810.2 | −12.4 |
| SUODN | PROKR2 | Antagonist | 2.5 | 11640 | 12640 | 12140.00 | 707.1 | −17.5 |
| SUODN | PTAFR | Antagonist | 2.5 | 445960 | 588840 | 517400.00 | 101031.4 | −7.3 |
| SUODN | PTGER2 | Antagonist | 2.5 | 20480 | 18200 | 19340.00 | 1612.2 | 2.1 |
| SUODN | PTGER3 | Antagonist | 2.5 | 131160 | 151200 | 141180.00 | 14170.4 | −10 |
| SUODN | PTGER4 | Antagonist | 2.5 | 125840 | 117320 | 121580.00 | 6024.6 | −15.1 |
| SUODN | PTGFR | Antagonist | 2.5 | 16760 | 18000 | 17380.00 | 876.8 | −8.4 |
| SUODN | PTGIR | Antagonist | 2.5 | 86520 | 81280 | 83900.00 | 3705.2 | −20 |
| SUODN | PTHR1 | Antagonist | 2.5 | 659000 | 631160 | 645080.00 | 19685.8 | −5.7 |
| SUODN | PTHR2 | Antagonist | 2.5 | 423800 | 430920 | 427360.00 | 5034.6 | 4.5 |
| SUODN | RXFP3 | Antagonist | 2.5 | 19800 | 19680 | 19740.00 | 84.8 | 31.4 |
| SUODN | SCTR | Antagonist | 2.5 | 354160 | 317120 | 335640.00 | 26191.2 | 7.7 |
| SUODN | SSTR1 | Antagonist | 2.5 | 3920 | 3480 | 3700.00 | 311.1 | −1.6 |
| SUODN | SSTR2 | Antagonist | 2.5 | 61400 | 63960 | 62680.00 | 1810.2 | −20.5 |
| SUODN | SSTR3 | Antagonist | 2.5 | 137800 | 143880 | 140840.00 | 4299.2 | 4.2 |
| SUODN | SSTR5 | Antagonist | 2.5 | 86040 | 79880 | 82960.00 | 4355.8 | −17.3 |
| SUODN | TACR1 | Antagonist | 2.5 | 838600 | 790120 | 814360.00 | 34280.5 | −17.6 |
| SUODN | TACR2 | Antagonist | 2.5 | 141560 | 124560 | 133060.00 | 12020.8 | 8.3 |
| SUODN | TACR3 | Antagonist | 2.5 | 144760 | 157080 | 150920.00 | 8711.6 | −5.8 |
| SUODN | TBXA2R | Antagonist | 2.5 | 89600 | 90400 | 90000.00 | 565.7 | 16.2 |
| SUODN | TRHR | Antagonist | 2.5 | 11320 | 13840 | 12580.00 | 1781.9 | −6.6 |
| SUODN | TSHR(L) | Antagonist | 2.5 | 4560 | 4800 | 4680.00 | 169.7 | −19.1 |
| SUODN | UTR2 | Antagonist | 2.5 | 17800 | 17960 | 17880.00 | 113.1 | −27.2 |
| SUODN | VIPR1 | Antagonist | 2.5 | 407880 | 387440 | 397660.00 | 14453.3 | −11.6 |
| SUODN | VIPR2 | Antagonist | 2.5 | 442360 | 396720 | 419540.00 | 32272.4 | −6.7 |

While certain features of the invention have been described herein, many modifications, substitutions, changes. and equivalents will now occur to those of ordinary skill in the art. It is, therefore, to be understood that the appended claims are intended to cover all such modifications and changes as fall within the scope of the invention.

What is claimed is:

1. An isolated or purified octadecaneuropeptide (ODN) or synthetic derivative thereof, comprising an amino acid sequence selected from one or more of SEQ ID NOS:1 to SEQ ID NO: 21 or a functional fragment thereof, or a sequence having at least 95% identity thereto, said octadecaneuropeptide modulating food intake, and body weight.

2. The octadecaneuropeptide of claim 1, wherein said octadecaneuropeptide inhibits emesis and nausea.

3. The octadecaneuropeptide of claim 1, further comprising one or more of
i) modified amino acids listed in Table 2, and, or
ii) Gln(alkyn), Ala(Alkyn), Gly(Alkyn) and Lys(N3).

4. The octadecaneuropeptide of claim 3, wherein said modified amino acid facilitates bioconjugation of an agent selected from one or more of a N and, or C terminus protection moiety, a lipid, a recombinant FC-peptide, a cell penetrating peptide, and enhances one or more of peptide function, stability, and bioavailability.

5. The octadecaneuropeptide of claim 1, which is a synthetic derivative of SEQ ID NO:3 or a functional modified variant thereof.

6. A composition comprising the octadecaneuropeptide of claim 1 in a pharmaceutically acceptable carrier or buffer.

7. A nucleic acid sequence that encodes any one of the octadecaneuropeptides having amino acid SEQ ID NOS: 1 to SEQ ID NO: 21 of claim 1.

8. The nucleic acid of claim 7, which encodes the polypeptide of SEQ ID NO: 3.

9. The nucleic acid of claim 7, encapsulated in a liposome or an extracellular vesicle or affixed to a nanoparticle or lipid nanoparticle.

10. A vector comprising the nucleic acid of claim 7.

11. The vector of claim 10, which is a) a plasmid vector, a lentiviral vector, an AAV vector, AAV9, and AAV8 or b) encapsulated in a liposome or an extracellular vesicle or affixed to a nanoparticle or lipid nanoparticle.

12. (canceled)

13. A composition comprising the nucleic acid encoding the octadecaneuropeptide of claim 1 in a pharmaceutically acceptable carrier or buffer.

14. A method of treating a metabolic disease or disorder in a patient in need thereof, comprising administering an effective amount of the composition of claim 6.

15. The method of claim 14, wherein the metabolic disease or disorder is selected from obesity, diabetes mellitus, type 1 diabetes, type 2 diabetes, dyslipidemia, insulin resistance, hepatic steatosis, hypercholesterolemia, and non-alcoholic fatty liver.

16. (canceled)

17. The method of claim 1, further comprising a) administering a second therapeutic agent that treats or inhibits progress at least one disorder selected from obesity, diabetes mellitus, dyslipidemia, insulin resistance, hepatic steatosis, hypercholesterolemia, and non-alcoholic fatty liver or b providing the patient with lifestyle intervention on diet and/or physical activity on said subject.

18. (canceled)

19. The method of claim 14, wherein a) the patient has a reduced food intake for 1, 3, 6, and/or 24 hours after administration of said peptide or nucleic acid encoding said peptide when compared to an untreated control or b) the weight of the patient decreases following administration of the peptide.

20. (canceled)

21. The method of claim 14, further comprising assessing the patient for a reduction in symptoms associated with at least one disorder selected from obesity, diabetes mellitus, dyslipidemia, insulin resistance, hepatic steatosis, hypercholesterolemia, and non-alcoholic fatty liver.

22. The method of claim 14, wherein a) said composition or carrier is formulated to cross the blood brain barrier and administration is via an intracranial, intraparenchymal, cerebro-ventricular, or transmeningeal route or b) administration of said peptide is via a route selected from systemic, parenteral, transdermal patch, intramuscular, subcutaneously, buccal swab and oral.

23. (canceled)

24. The method of claim 14, wherein said composition is effective inhibit relaxin-3 receptor producing anorexic effects.

25. The method of claim 14 wherein said composition functions to modulate one or more of;
i) improve glucose tolerance;
ii) modulate relaxin-3 receptor activity;
iii) regulate the counterregulatory response and attenuate 5-tg induced hyperglycemia;
iv) improve insulin sensitivity;
v) promote weight loss; and
v) penetrate into the brain.


SEQUENCE LISTING (29 sequences)

SEQ ID NO: 1 - protein, 18 aa, synthetic construct
QATVGDVNTDRPGLLDLK

SEQ ID NO: 2 - protein, 8 aa, synthetic construct
  SITE 5: note = D-Leucine
RPGLLDLK

SEQ ID NO: 3 - protein, 13 aa, synthetic construct
DVNTDRPGLLDLK

SEQ ID NO: 4 - protein, 8 aa, synthetic construct
RPGLLDLK

SEQ ID NO: 5 - protein, 5 aa, synthetic construct
RPGLL

SEQ ID NO: 6 - protein, 8 aa, synthetic construct
DVNTDRPG

SEQ ID NO: 7 - protein, 10 aa, synthetic construct
DVNTDRPGLL

SEQ ID NO: 8 - protein, 13 aa, synthetic construct
QATVGDVNTDRPG

SEQ ID NO: 9 - protein, 15 aa, synthetic construct
QATVGDVNTDRPGLL

SEQ ID NO: 10 - protein, 18 aa, synthetic construct
  MOD_RES 1: note = Glutamine (alkyn)
  MOD_RES 18: note = N-Fmoc-6-azidonorleucine
QATVGDVNTDRPGLLDLK

SEQ ID NO: 11 - protein, 18 aa, synthetic construct
  MOD_RES 1: note = Glutamine (alkyn)
  MOD_RES 14: note = N-Fmoc-6-azidonorleucine
QATVGDVNTDRPGKLDLK

SEQ ID NO: 12 - protein, 18 aa, synthetic construct
  MOD_RES 1: note = Glutamine (alkyn)
  MOD_RES 14: note = N-Fmoc-6-azidonorleucine
QATVGDVNTDRPGLKDLK

SEQ ID NO: 13 - protein, 18 aa, synthetic construct
  MOD_RES 1: note = Glutamine (alkyn)
  MOD_RES 17: note = N-Fmoc-6-azidonorleucine
QATVGDVNTDRPGLLDKK

SEQ ID NO: 14 - protein, 18 aa, synthetic construct
  MOD_RES 2: note = Alanine (alkyn)
  MOD_RES 18: note = N-Fmoc-6-azidonorleucine
QATVGDVNTDRPGLLDLK

SEQ ID NO: 15 - protein, 18 aa, synthetic construct
  MOD_RES 2: note = Alanine (alkyn)
  MOD_RES 14: note = N-Fmoc-6-azidonorleucine
QATVGDVNTDRPGKLDLK

SEQ ID NO: 16 - protein, 18 aa, synthetic construct
  MOD_RES 2: note = Alanine (alkyn)
  MOD_RES 15: note = N-Fmoc-6-azidonorleucine
QATVGDVNTDRPGLKDLK

SEQ ID NO: 17 - protein, 18 aa, synthetic construct
  MOD_RES 2: note = Alanine (alkyn)
  MOD_RES 17: note = N-Fmoc-6-azidonorleucine
QATVGDVNTDRPGLLDKK

SEQ ID NO: 18 - protein, 18 aa, synthetic construct
  MOD_RES 5: note = Glycine (alkyn)
  MOD_RES 18: note = N-Fmoc-6-azidonorleucine
QATVGDVNTDRPGLLDLK

SEQ ID NO: 19 - protein, 18 aa, synthetic construct
  MOD_RES 5: note = Glycine (alkyn)
  MOD_RES 14: note = N-Fmoc-6-azidonorleucine
QATVGDVNTDRPGKLDLK

SEQ ID NO: 20 - protein, 18 aa, synthetic construct
  MOD_RES 5: note = Glycine (alkyn)
  MOD_RES 15: note = N-Fmoc-6-azidonorleucine
QATVGDVNTDRPGLKDLK

SEQ ID NO: 21 - protein, 18 aa, synthetic construct
  MOD_RES 17: note = N-Fmoc-6-azidonorleucine
  MOD_RES 5: note = Glycine (alkyn)
QATVGDVNTDRPGLLDKK

SEQ ID NO: 22 - protein, 16 aa, Drosophila melanogaster
RQIKIWFQNRRMKWKK

SEQ ID NO: 23 - protein, 33 aa, Human alphaherpesvirus 1
DAATATRGRSAASRPTERPRAPARSASRPRRVD

SEQ ID NO: 24 - protein, 18 aa, synthetic construct
KLALKLALKALKAALKLA

SEQ ID NO: 25 - protein, 27 aa, synthetic construct
GWTLNSAGYLLGKINLKALAALAKKIL

SEQ ID NO: 26 - protein, 7 aa, synthetic construct
RRRRRRR

SEQ ID NO: 27 - protein, 23 aa, synthetic construct
GALFLGWLGAAGSTMGAPKKRKV

SEQ ID NO: 28 - protein, 21 aa, synthetic construct
KETWWETWWTEWSQPKKKRKV

SEQ ID NO: 29 - protein, 11 aa, Human immunodeficiency virus 1
YGRKKRRQRRR